(19) United States
(12) Patent Application Publication
(10) Pub. No.: US 2024/0094157 A1
(43) Pub. Date: Mar. 21, 2024

(54) POINT OF CARE SALIVARY TESTING DEVICES AND METHODS

(71) Applicant: TRAQ, Inc., Carlsbad, CA (US)

(72) Inventors: Jeff SLOSAR, Rancho Santa Fe, CA (US); Andres DUVVURI, San Marcos, CA (US); Thomas CHO, San Marcos, CA (US); Manian Pillai MANESH KALAYIL, San Diego, CA (US); Santhosh PADMANABHAN, San Diego, CA (US); Natalie SLOSAR, Rancho Santa Fe, CA (US); Kelly SLOSAR, Rancho Santa Fe, CA (US); Avery SLOSAR, Rancho Santa Fe, CA (US); Jackson SLOSAR, Rancho Santa Fe, CA (US)

(21) Appl. No.: 18/471,554

(22) Filed: Sep. 21, 2023

Related U.S. Application Data
(60) Provisional application No. 63/376,594, filed on Sep. 21, 2022.

(51) Int. Cl. G01N 27/327; G01N 1/40; G16H 40/67

(52) U.S. Cl. CPC G01N 27/3273; G01N 1/405; G01N 27/3272; G16H 40/67

(57) ABSTRACT

According to some embodiments, a point of care salivary testing device comprises a biofluid collection device, a cartridge device, a reader device, and/or a software-based detection interface system. The analyte detection system may be used to detect the presence and/or quantity of one or more target analytes.

### RELATED APPLICATIONS

The present application claims priority to U.S. App. Ser. No. 63/376,594 filed Sep. 21, 2022, incorporated herein by reference in its entirety.

### FIELD OF THE DISCLOSURE

The present disclosure relates to point of care salivary testing devices and their uses.

### BACKGROUND OF THE DISCLOSURE

Determining and/or gaining information about a person's fitness level, or general health & wellness, and/or obtaining diagnostic information, is expensive, invasive and/or inconvenient, e.g., requiring a visit a physician.

For fitness determinations, solutions include wearable devices, step counters, as well as more involved treadmill and VO2 max tests. For general health and wellness determinations, blood tests and mail-in tests using blood, saliva, stool, urine are used. For diagnostic determination of a condition, blood tests and specific scans may be required (CAT, X-ray, MRI, etc.).

For fitness determinations, the current solutions are either only proxies for true health (step counters, wearables) or are very specific to one form of health and are also very expensive (treadmill, VO2 max tests). Apart from wearables, professionals are currently needed to measure fitness, health & wellness, or a diagnostic biomarker. General health and wellness testing through blood tests at the doctor's office are expensive, invasive, and inconvenient. Mail-in test kits, while more convenient, are still slow and expensive and could still be invasive. Lastly, diagnostic determination of a condition (blood tests, MRI/PET/CAT/X-ray scans, etc.), while considered the gold standard for sensitivity and specificity or a condition, are very expensive, inconvenient, and invasive. Further, any physical exams performed by healthcare providers are also expensive, invasive and/or inconvenient.

### SUMMARY

According to some embodiments, a point-of-care testing (POCT) system is provided. An example POCT system, among other example benefits, can allow for on-demand, portable, non-invasive (saliva) testing for fitness, general health and/or wellness and/or may be used as a diagnostic tool for detecting severe maladies. Example POCT systems can provide results e.g., on the order of seconds to minutes, and can provide results in a less costly manner. The POCT system can be operated without the need of a medical professional or otherwise.

According to some embodiments, a point-of-care testing (POCT) system allows for on-demand, portable, non-invasive (saliva) testing for fitness, general health and/or wellness and/or may be used as a diagnostic tool for detecting maladies, with results available in seconds to minutes, and costing only a few dollars per test without the need of a medical professional or otherwise.

According to some embodiments, a point of care salivary testing device comprises a biofluid collection device, a cartridge device, a reader, and/or a software-based detection interface system. The analyte detection system may be used to detect the presence and/or quantity of one or more target analytes.

According to some embodiments, a biofluid collection device intended for collecting a biofluid comprises a handle and an absorbent tip. According to some embodiments the biofluid collection device further comprises coupling mechanism by which the device can be coupled to a cartridge base. According to some embodiments the biofluid collection device further comprises an extendable member. According to some embodiments, the extendable member is a telescoping arm.

According to some embodiments, an electrochemically active sensor chip, comprises a substrate including an electrically insulative material; a plurality of electrode wells disposed on the substrate in different locations, wherein each well includes a plurality of electrodes containing a surface including a chemical substance that includes a catalyst or reactant corresponding to an analyte in saliva; wherein the electrodes are capable of sustaining a redox reaction with the chemical substance and analyte to produce an electrical signal. According to some embodiments, the sensor chip of further comprises a spacer layer coupled to the substrate, which allows saliva to pass into the wells; and a hydrophilic top cover, which directs saliva towards the wells and seals the chip.

According to some embodiments, an electrochemically active sensor chip, comprises a substrate including an electrically insulative material; and a plurality of electrode wells disposed on the substrate in different locations, wherein each well includes a plurality of electrodes containing a surface including a chemical substance that includes a catalyst or reactant corresponding to an analyte in saliva; wherein the electrodes are capable of sustaining a redox reaction with the chemical substance and analyte to produce an electrical signal. According to some embodiments, the sensor chip further comprises a spacer piece coupled to the substrate, which allows saliva to pass into the wells; and a hydrophilic top cover, which directs saliva towards the wells and seals the chip.

According to some embodiments, a cartridge base comprises a cartridge body defining a collection device cavity therein, wherein a first side of the cartridge body has an opening to the cavity, wherein a portion of a biofluid collection device is configured to be inserted in the collection device cavity of the cartridge body; and the cartridge body defining a sensor cavity; and a biosensor chip in the sensor cavity.

According to some embodiments, a reader unit comprises electrical components for wireless data transmission; a potentiostat; and a battery; wherein the reader unit is configured to collect data, wherein the reader unit is configured to interface with electrodes through contact with a cartridge.

According to some embodiments, a reader unit comprising: a cartridge receptacle configured to receive therein a cartridge, the cartridge having a biosensor therein, the biosensor configured to generate one or more electrical signals in response to a biofluid contacting the biosensor; and a processor communicatively coupled to the biosensor of a cartridge received in the cartridge receptable and configured to determine concentrations of one or more biomarkers in the biofluid.

According to some embodiments, a point of care salivary testing system comprises a biofluid collection device configured to collect a biofluid; a cartridge base configured to receive the collection device, the cartridge base having a biosensor therein, wherein the cartridge base is configured to transfer collected biofluid from the collection device to the biosensor after the collection device having a collected biofluid thereon is received within the cartridge base, the biosensor having a plurality of electrical pads thereon; and a reader device configured to receive the cartridge base therein, wherein the reader device has a plurality of electrical contacts configured to become electrically coupled to the electrical pads of the biosensor when the cartridge base is received in the reader device, wherein the system is configured to detect the presence and/or quantity of one or more target analytes in the collected biofluid that has been transfer to the biosensor that is electrically coupled to the electrical contacts of the reader.

According to some embodiments, a method to detect an analyte in saliva comprises reducing a chemical substrate in the saliva to a second product in a chemical reduction process in which the second product gains electrons; supplying the extracted electrical energy to electrodes of an electrochemical sensor; and detecting, from the electrodes of the electrochemical sensor in contact with the saliva, an electrical signal produced as a result of a redox reaction involving an analyte in the saliva and a chemical agent coupled to at least one of the electrodes of the electrochemical sensor.

The above summary is not intended to represent every embodiment or every aspect of the present disclosure. Rather, the foregoing summary merely provides an exemplification of some of the novel aspects and features set forth herein. The above features and advantages, and other features and advantages of the present disclosure, which are inventive singly or in any combination, will be readily apparent from the following detailed description of representative embodiments and modes for carrying out various embodiments of the present disclosure when taken in connection with the accompanying drawings and the appended claims.

### BRIEF DESCRIPTION OF THE DRAWINGS

FIGS. 1A-1E illustrate a Point-of-Care Testing (“POCT”) system according to some embodiments of the present disclosure.

FIG. 2 illustrates a biosensor chip according to some embodiments of the present disclosure.

FIG. 3 illustrates an exemplary step-by-step protocol for using a POCT system, which includes a handheld device and disposable biofluid or saliva test cartridge, according to some embodiments of the present disclosure.

FIG. 4A is a perspective view of a disposable biofluid or saliva test cartridge showing both a biofluid or saliva collection device in an extended mode or position and a cartridge base according to some embodiments of the present disclosure.

FIG. 4B is a perspective view the disposable biofluid or saliva test cartridge with a biofluid or saliva collection device in a collapsed position or mode and inserted into the cartridge base according to some embodiments of the present disclosure.

FIG. 4C illustrates an example biofluid or saliva collection device, with an enlarged view of an interior portion of a telescoping arm according to some embodiments of the present disclosure.

FIG. 5A illustrates a person's head with a biofluid or saliva collection device inserted into an oral cavity of the person with a tip of collection device contacting near the parotid gland and duct according to some embodiments of the present disclosure.

FIG. 5B illustrates an exterior view of a person's head with a saliva collection device inserted into an oral cavity of the person as shown in FIG. 5A with a handle of the collection device positioned outside the oral cavity according to some embodiments of the present disclosure.

FIG. 6A-6F are views of a biofluid or saliva collection handle according to some embodiments of the present disclosure.

FIG. 7A is an exploded perspective view and FIG. 7B is an assembled perspective view of a cartridge base according to some embodiments of the present disclosure.

FIG. 7C is a perspective view of a latch for the cartridge base according to some embodiments of the present disclosure.

FIGS. 7D-7I are views of a cartridge base (cartridge body) according to some embodiments of the present disclosure.

FIG. 8A is an exploded perspective view of a subassembly of a disposable biofluid or saliva test cartridge or kit showing a collection device (with the telescoping arm omitted), a cartridge base, a biosensor chip, and a cartridge latch.

FIG. 8B is an assembled perspective view of a biofluid or saliva test cartridge according to some embodiments of the present disclosure, illustrating an example method for packaging the subassembly before use and during analysis.

FIGS. 8C-8D are assembled perspective cross-sectional views and FIG. 8F is an unassembled side, cross-sectional view of a disposable biofluid or saliva test cartridge according to some embodiments of the present disclosure.

FIG. 8E is a top view of the biosensor chip according to some embodiments of the present disclosure.

FIG. 8G is a perspective view of a cartridge containing a collection device that does not employ a telescoping arm according to some embodiments of the present disclosure.

FIG. 9A is an exploded perspective view of a collection device, a cartridge base, and a reader which may take the form of a handheld POCT testing device according to some embodiments of the present disclosure.

FIG. 9B is a cross-sectional view of a reader/POCT testing device with a cartridge inserted in a cartridge receptacle according to some embodiments of the present disclosure.

FIG. 9C is an exploded view of a reader/testing device and some of its component parts according to some embodiments of the present disclosure.

FIG. 9D is a perspective view of a cartridge receptacle having an aperture therein according to some embodiments of the present disclosure.

FIGS. 10-12 are plan views of PCB (printed circuit board) schematics according to some embodiments of the present disclosure.

FIG. 13A is a top view of a biosensor chip according to some embodiments of the present disclosure.

FIG. 13B is an exploded perspective view of a sensor chip showing various constituent layers according to some embodiments of the present disclosure.

FIG. 13C shows a top or plan view of screen-printed electrode layers of a biosensor chip according to some embodiments of the present disclosure.

FIG. 13D is an exploded perspective view of the screen-printed electrode layers and a dielectric layer of a biosensor chip according to some embodiments of the present disclosure. The screen-printed electrode layers and the dielectric layer are referred to collectively as a sensor chip base.

FIG. 13E is a top or plan view of a sensor chip base, shown in FIG. 13D according to some embodiments of the present disclosure.

FIG. 13F is a side schematic view illustrating various screen-printed layers of a sensor chip base according to some embodiments of the present disclosure.

FIG. 14A is a layer stackup of an enzymatic sensing film for a lactate sensing film, or an enzymatic sensing film generally according to some embodiments.

FIGS. 14B-14C shows an embodiment of a biosensor chip for measuring lactate and a general schematic of the reactions used according to some embodiments.

FIG. 15A is a layer stack up of an ion selective hydration sensing film for a hydration sensor, or an ion selective electrode generally (ISE) according to some embodiments.

FIG. 15B is a graph illustrating the stability response of the sensor to ion-selective reactions, by measuring different concentrations of NaCl over a 1-minute period according to some embodiments.

FIG. 15C-15E are graphs illustrating a response of a sensor at different concentrations of electrolytes, with FIG. 15C showing Potassium, FIG. 15D showing Sodium and FIG. 15E showing Chloride, according to some embodiments.

FIG. 15F is functional block diagram of a reader/testing device and a sensor according to some embodiments of the present disclosure.

FIG. 16A is a top or plan view of a strip which comprises a plurality of screen-printed pad layers, used for automated manufacturing according to some embodiments of the present disclosure.

FIG. 16B is a top or plan view of a strip of a spacer, used for automated manufacturing according to some embodiments of the present disclosure.

FIG. 16C is a top or plan view of a strip of a top cover, used for automated manufacturing according to some embodiments of the present disclosure.

FIG. 17A is an exploded perspective view of a component of an immunoassay system according to some embodiments of the present disclosure.

FIG. 17B is an exploded perspective view of components of a Cube Reader of an immunoassay system according to some embodiments of the present disclosure.

FIG. 17B′ is an exploded perspective view of components of a Cube Reader of an immunoassay system according to some other embodiments of the present disclosure.

FIG. 17C is a perspective view of a test bed according to some embodiments.

FIG. 17D is a perspective view of a magnetic gear assembly having magnetic gears according to some embodiments.

FIG. 17E is a perspective view of a manifold according to some embodiments.

FIG. 17F is a schematic depicting fluidic flow in a manifold and a microfluidics chip according to some embodiments.

FIG. 18A is an exploded perspective view of an immunoassay biosensor chip according to some embodiments.

FIG. 18B is a plan view of a biofluid, e.g., saliva, collector adapter for receiving a biofluid collection device according to some embodiments.

FIGS. 18C-18E are top views of a multiple layers of an immunoassay biosensor chip.

FIGS. 19A-19B and 19E-19F are top views of a multiple layers of an immunoassay biosensor chip according to some embodiments.

FIGS. 19C-19D shows example heating PCBs illustrating example wax valves thereon.

FIG. 19G1 is a three-dimensional perspective view illustrating microfluidic paths and wells as within a disposable microfluidics chip according to some embodiments.

FIG. 19G2 is a block diagram representing the physical flow of fluids through FIG. 19G1.—

FIG. 19H is a perspective bottom view, FIG. 19I is a top view, and FIG. 19J is a bottom view of an assembled microfluidics chip according to some embodiments.

FIG. 19K is functional block diagram of electrical components of an immunoassay system 1700 according to some embodiments of the present disclosure.

FIG. 20 is a flowchart of a general immunoassay process performed during analysis within an immunoassay device according to some embodiments.

FIG. 21 is a flowchart of chemical processes within an immunoassay device according to some embodiments.

FIG. 22 is a graph of the electrochemical reaction of cortisol according to some embodiments of the present disclosures.

FIG. 23 is a graph of a sensor response to the different concentrations of cortisol according to some embodiments of the present disclosures.

FIGS. 24A-24C are a flowchart illustrating a method of testing according to some embodiments of the present disclosure.

FIG. 25A-25C are a flowchart of the software illustrating a method of testing according to some embodiments of the present disclosure.

FIGS. 26A-26F and 2611-26P illustrate exemplary displays on a POCT device and/or a remote device such as a smartphone according to some embodiments of the present disclosure.

FIG. 26G illustrates the collection of a biofluid such as saliva according to some embodiments of the present disclosure.

FIGS. 27A-27E are example screen shots of a smartphone running an application (App) according to some embodiments of the present disclosure.

FIG. 28A shows various devices associated with different tests or general health categories and associated scores are displayed on the display of the device and provides examples of qualitative health categories with sample related underlying biomarkers according to some embodiments of the present disclosure.

FIG. 28B illustrates a POCT device communicating test results and scores to a smart phone according to some embodiments of the present disclosure.

FIG. 29A is a graph of an accuracy study from a POCT sensor configured to measure lactate, according to some embodiments of the present disclosure, as compared to an industry standard lactate concentration analyzer from YSI (YSI benchtop analyzer).

FIG. 29B is a graph showing the salivary lactate response to incremental physical exercise according to some embodiments of this disclosure.

FIG. 29C is a graph of a stability study by plotting comparing measurements obtained using a POCT sensor configured to measure lactate on Day 1 when the sensor is manufactured, up to Day 260 after the sensor is manufactured according to some embodiments.

FIG. 29D is a graph showing lot-to-lot manufacturing variability and robustness by plotting measured biomarker concentrations in three different production lots and applied to biosensor chips according to some embodiments.

FIG. 30 illustrates disposable biofluid or saliva test cartridges for various potential fitness categories (Nutrition, Energy, Burn, Hydration), etc.). of the present disclosure.

FIG. 31 illustrates disposable biofluid or saliva test cartridges for health & wellness categories (i.e. aging, defense, mood, GI Health, stress) or gender-based cartridges (e.g. women's health) of the present disclosure.

FIG. 32 illustrates disposable biofluid or saliva test cartridges for specific maladies or medical conditions (e.g., acidosis) or personalized medicine with dedicated cartridges for an individual (e.g., “John Doe”) according to some embodiments of the present disclosure.

FIGS. 33A-33C illustrate a table of examples of Health Categories & exemplary related, underlying biomarkers according to some embodiments of the present disclosure.

FIG. 34A illustrates some screens on a smartphone showing a health category score for Hydration shown on mobile application (left), along with related underlying biomarker concentrations (middle) and historical scores (right), according to some embodiments of the present disclosure.

FIG. 34B shows a mobile software application displaying underlying biomarker concentration for Hydration heath category (left), and self-directed actionable insights (middle, right) according to some embodiments of the present disclosure.

FIG. 34C shows a mobile software application displaying self-directed actionable insights (left, right) and external link to advisors, coaches, medical professionals, or others (center) according to some embodiments of the present disclosure.

The present disclosure is susceptible to various modifications and alternative forms, and some representative embodiments have been shown by way of example in the drawings and will be described in detail herein. It should be understood, however, that the inventive aspects are not limited to the forms illustrated in the drawings. Rather, the disclosure is to cover all modifications, equivalents, combinations, and alternatives falling within the spirit and scope of the inventions as defined by the appended claims.

### DETAILED DESCRIPTION OF THE ILLUSTRATED EMBODIMENTS

The terminology “about” is meant to designate a possible variation of up to ±10%. Terms of orientation such as upper, lower, top, bottom, left, or right are included in this disclosure to aid understanding of the disclosure such as in reference to one or more of the drawings but at are not limitations as it is understood that the orientation of objects and surfaces described herein may be altered and hence the relative orientation terms may likewise be altered.

Words of orientation such as up, down, top, bottom, left, right, are exemplary and are made with reference to various figures of the present disclosure and are used to aid in understanding of the various embodiments disclosed therein. However, it is understood that the orientation of the components and devices described herein may be changed, e.g., a device may be turned or positioned upside down relative to what is shown in a particular figure, and hence what may be referred to as a top of a component or device may become positioned below what may have been referred to as a bottom of a component or device.

Various devices, systems, kits, and methods disclosed herein are intended to detect a target analyte within a biofluid sample taken from a specimen. In certain embodiments, chemical reactions are employed to enable such detection.

### 1. Overview POCT System

According to some embodiments, a Point-of-Care Testing (“POCT”) system is provided to permit a biofluid of a person to be tested or analyzed and one or more characteristics of the biofluid to be determined and from which one or more characteristics about the person can be determined.

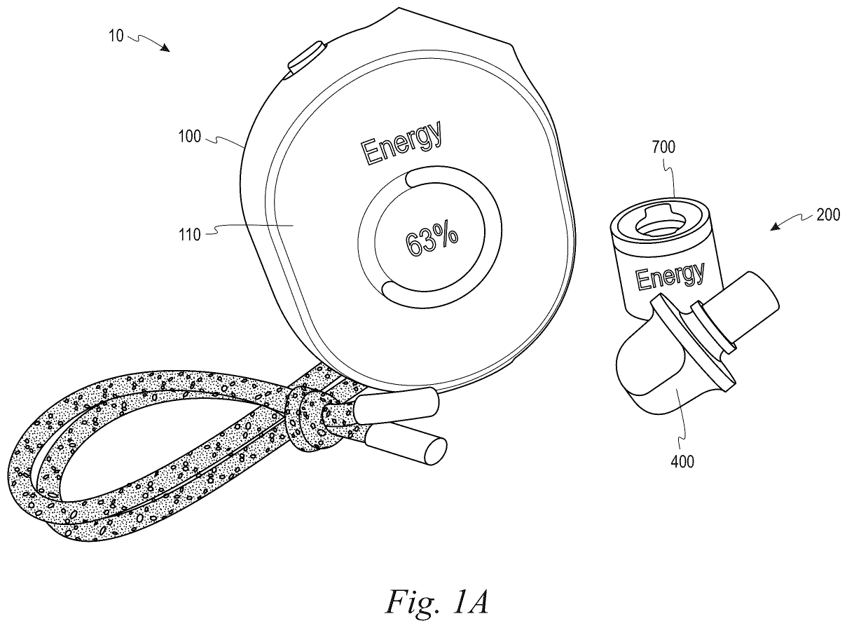

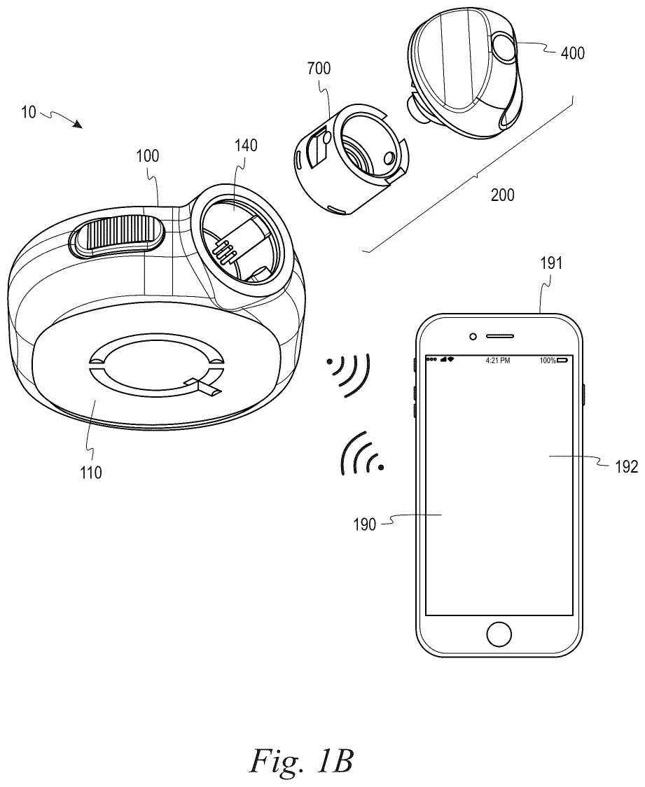

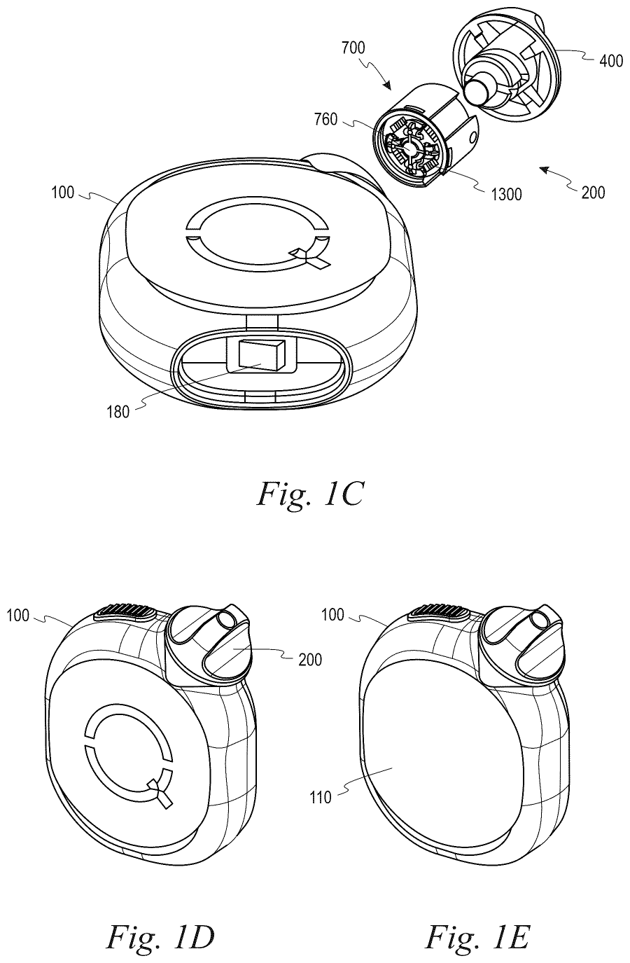

FIGS. 1A and 1B illustrates a Point-of-Care Testing (“POCT”) system 10 according to some embodiments of the present disclosure. According to some embodiments, the POCT system 10 comprises a handheld POCT device or “Stone” or reader 100 comprising a display 110. In FIG. 1A, the display 110 displays a score, in this case an “Energy” score of 63%. The system 10 also comprises one or more cartridges 200. For example, in FIG. 1A a single use cartridge 200 for measuring Energy is shown. Each cartridge 200 comprises a cartridge base 700 and a cartridge biofluid collection device 400. FIG. 1A is a perspective view of a POCT device 100 and a cartridge 200 of the system 10.

According to some embodiments, the POCT system 10 is comprised of a portable measuring and/or display device 100 and disposable testing cartridges 200. According to some embodiments, device 100 comprises electronics, a display 110, and/or communication mechanisms. According some embodiments, the POCT system 10 is designed to analyze saliva and the disposable cartridges each contain a means 400 of collecting a saliva sample and an embedded biosensor in the cartridge base 700 capable of sensing a characteristic of and/or concentrations of one or more substances in a sample of saliva, e.g., lactate concentration in saliva and/or other biofluids.

According to some embodiments, the Point-of-Care Testing (POCT) device 100 is called a “Stone”. According to some embodiments, the system 10 comprises the Stone 100 and a disposable cartridge 200. The system 10 uses a disposable cartridge 200 to both collect a saliva sample and transfer the sample to the cartridge base 700 and/or Stone 100. The system 10 measures, for example, salivary biomarkers (analytes, compounds, and/or elements) that may indicate, for example, a broader sense of health & wellness and/or may be used as a diagnostic screen as well, with FDA or other governmental approval.

According to some embodiments, the system 10 may be used to generate and provide a general health, fitness and/or wellness score, and/or an underlying biomarker concentration measurement.

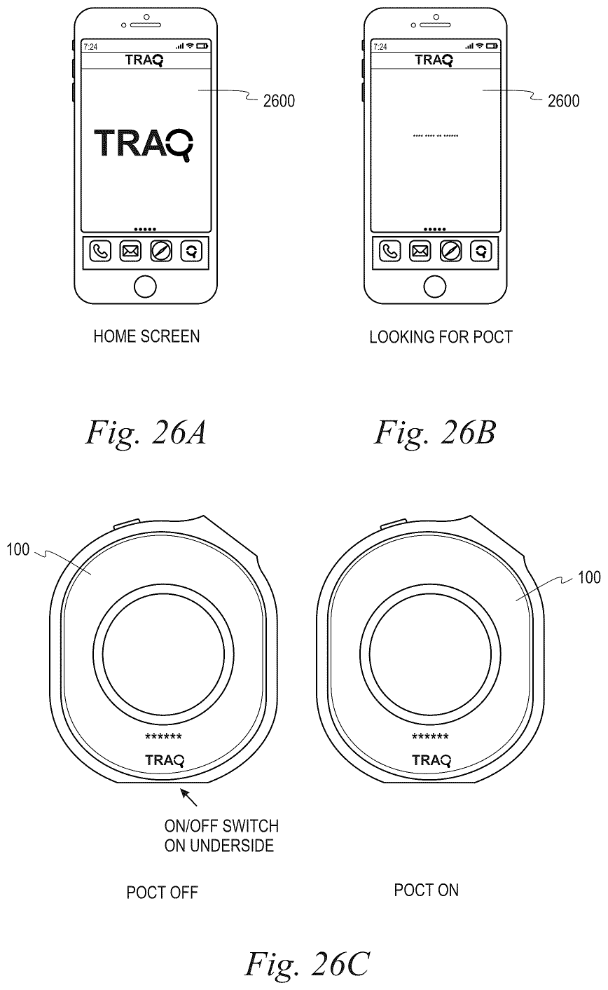

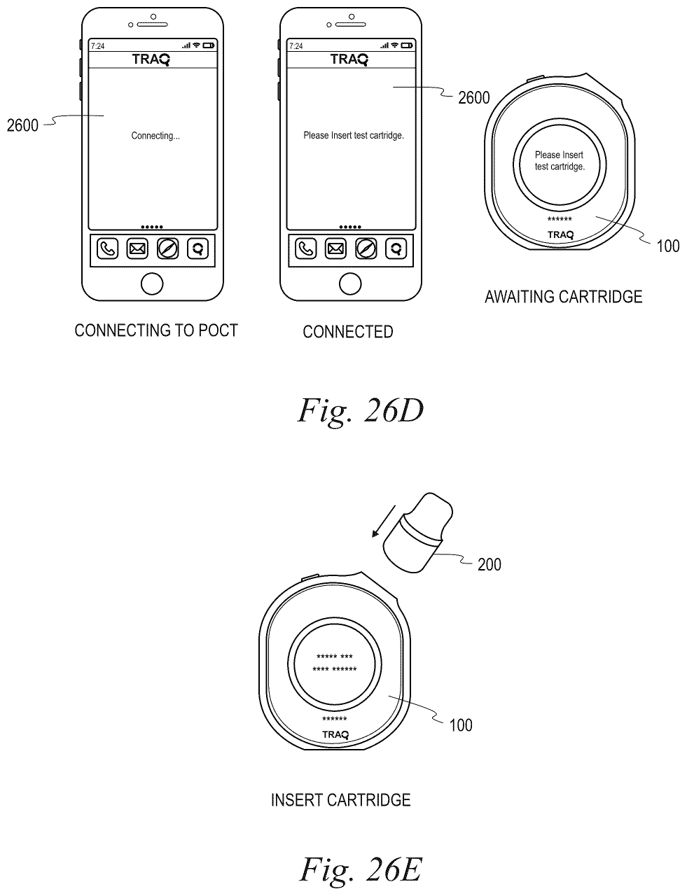

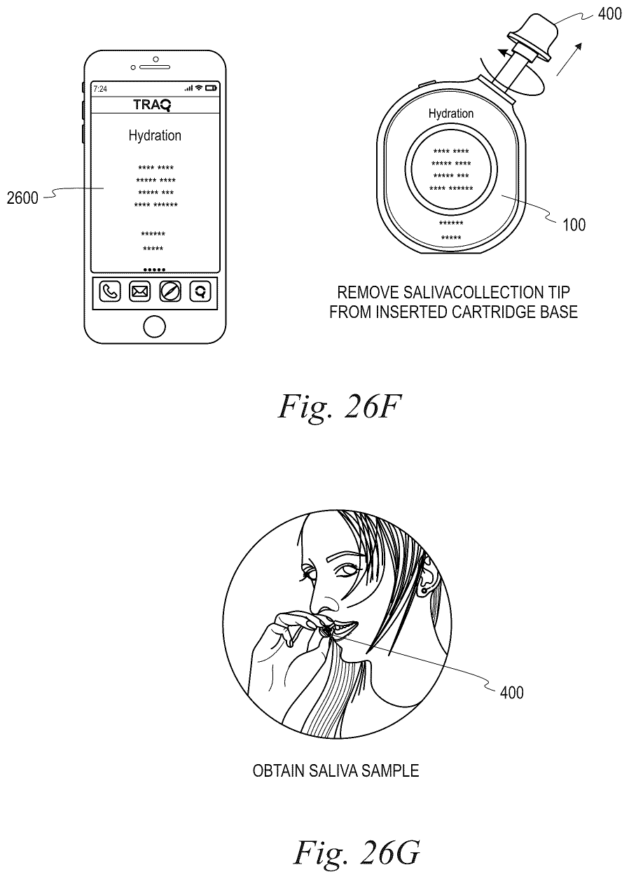

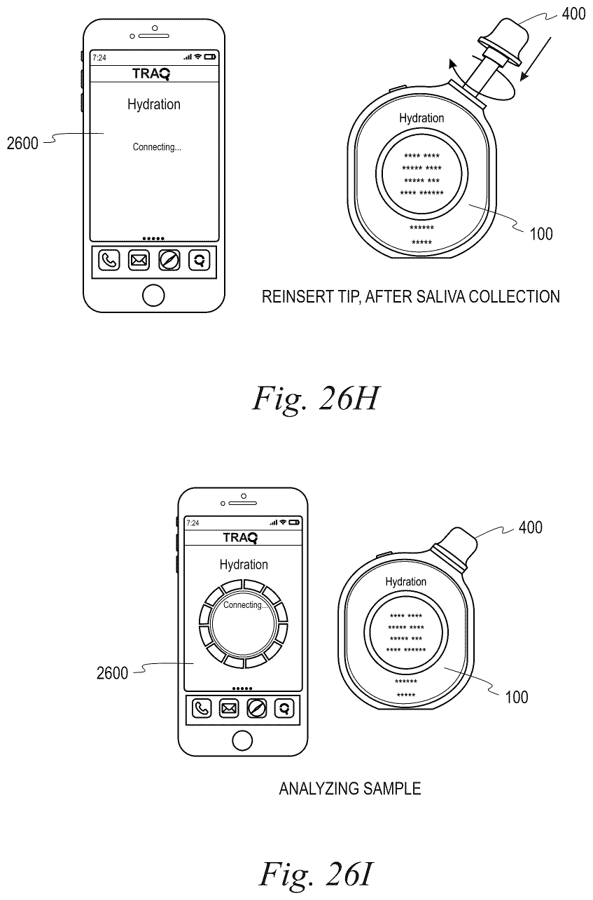

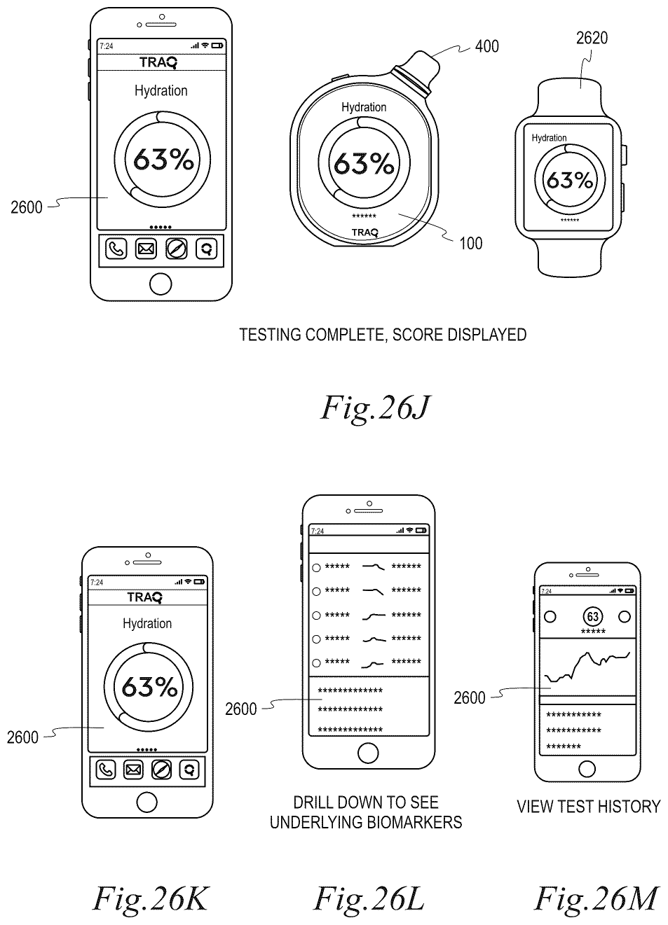

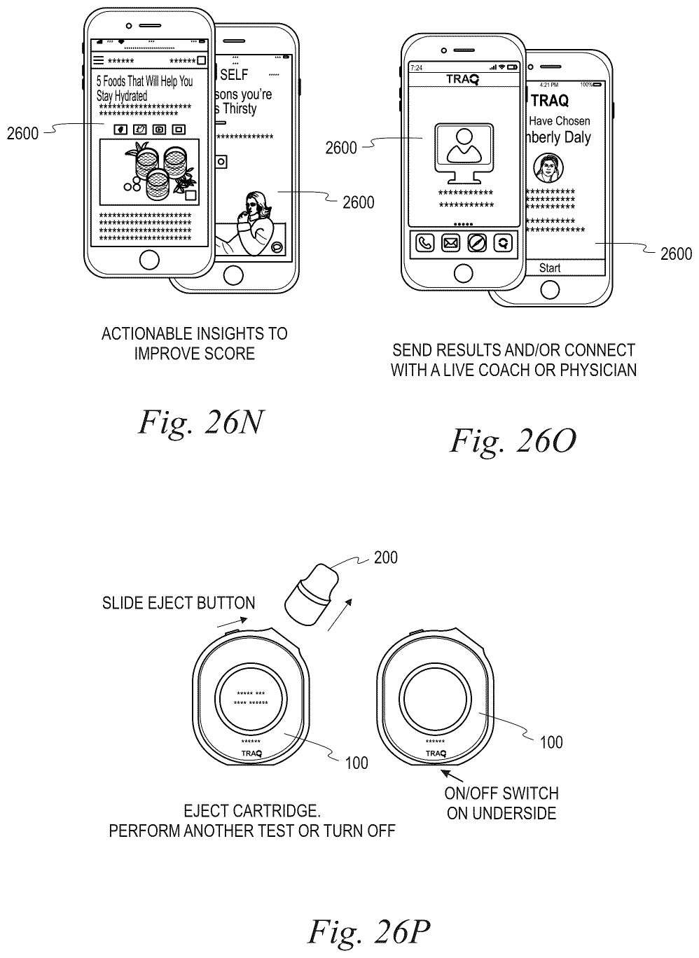

According to some embodiments, as shown in FIG. 1B, the POCT system 10 comprises three main components: the handheld Stone POCT device 100, a single-use cartridge 200 and optionally, a mobile or smartphone 191 or some other remote device (e.g., a smartwatch 2620 shown in FIG. 26J) with a related application (App”) installed, e.g., a TRAQ App. According to some embodiments, the App is not required for testing but may be required for updating the POCT device 100 and for more involved interaction with the internet and/or for sharing results.

FIG. 1B provides a perspective view of a POCT device 100 and a cartridge 200 of the system 10 according to some embodiments. FIG. 1B provides a perspective view of a first end of the POCT device or reader 100, e.g., a downward perspective view of a top end of the POCT device or reader 100. FIG. 1C provides a perspective view of a second, opposing end of the POCT device or reader 100, e.g., an upward perspective view of a bottom end of the POCT device or reader 100 and a de-coupled cartridge 200. FIG. 1D provides a perspective view of a first side of the POCT device or reader 100 and a cartridge 200 coupled thereto, e.g., inserted therein. FIG. 1E provides a perspective view of a second, opposing side of the POCT device or reader 100 and a cartridge 200 coupled thereto, e.g., inserted therein.

According to some embodiments, FIG. 1B illustrates an exemplary analyte detection system 10. The detection system 10 may include the biofluid collection device 400 cartridge base 700, the Stone device 100, and/or software-based detection interface system 190. The analyte detection system 10 may be used to detect the presence and/or quantity of one or more target analytes.

The collection device 400 may be exposed to biofluid samples, such as, but not limited to, blood, plasma, urine, saliva, mucous or other biofluids for determining the presence and/or quantity of one or more target analytes within a sample.

According to some embodiments, the cartridge base 700 is configured to analyze the sample collected with the collection device 400. As seen in FIG. 1C, cartridge base 700 may include a biosensor chip 1300 such that the collected sample may be analyzed within the cartridge base 700. Based on the analysis, cartridge base 700 is configured to generate electric signals indicative of the presence and/or quantity of one or more target analytes within the sample.

The Stone or reader 100 is configured for electric coupling with cartridge base 700 to permit transmission of the electric signals indicative of the presence and/or quantity of one or more target analytes within the sample generated by cartridge base 700 to the Stone 100 and/or to provide electrical signals and/or power to the cartridge base 700 and/or the chip 1300 therein. Cartridge base 700 may be electrically coupled to reader 100 by inserting a cartridge base 700 within a cartridge receptacle 140 in the Stone 100 as shown in FIG. 1B such that respective electrical connectors of cartridge base 700 and Stone 100 contact one another. According to some embodiments, the Reader 100 may comprise a computer readable medium with instructions that, when executed by a processor of reader 100, cause electrical components of reader 100 to perform steps for analyzing the sample on biofluid collection device 400 and/or receiving signals from the biosensor chip 1300 located in the cartridge base 700. According to some embodiments, the instructions are not executed until cartridge base 700 and/or chip 1300 is electrically coupled to reader 100 and biofluid collection device 400 is suitably disposed within cartridge base 700, for example, as shown in FIGS. 1D and 1E.

According to some embodiments, biofluid collection device 400 and cartridge base 700 are each disposable and designed for one time use while reader 100 is designed for multi-use and for receiving many different cartridge devices 200 throughout the life of the reader 100 such that many samples are analyzed by the reader 100 for determining the presence and/or quantity of one or more target analytes within the respective samples. Such a configuration is expected to promote sanitary use of the system 10, as the components exposed to a biofluid sample are disposable, while reducing costs as the components with more expensive electronics and parts, e.g., reader 100, may be used repeatedly.

As illustrated in FIG. 1B, a software-based detection interface system 190 may be installed and run on mobile a computing device 191 to permit a user to review analyte detection test results and actionable insights therein, e.g., on a display 192 of the computing device 191. The computing device 191 may be, for example, a smartphone, smartwatch, tablet, or wearable device or a computer such as a laptop. According to some embodiments, the computing device 191 is a mobile device. As shown in FIG. 1B, the reader 100 may communicate with computing device 191 wirelessly to transmit data indicative of the presence and/or quantity of one or more target analytes based on the electrical signals generated within cartridge base 700, though connections may alternatively or additionally be wired. Communication may be direct (e.g., direct wireless or wired communication) or indirect (e.g., via a server, a host device, or a cloud-based system). Software-based detection interface system 190 may comprise a computer readable medium with instructions that, when executed by a processor of the computing device 191, cause the display 192 to display information indicative of the presence and/or quantity of one or more target analytes.

In addition, or alternatively, the reader 100 can include a display 110, as shown in FIG. 1A. In such an embodiment, the reader 100 may comprise a computer readable medium with instructions that, when executed by a processor of reader 100, cause display 110 to display information indicative of the presence and/or quantity of one or more target analytes, and actionable insights thereof, and, in some embodiments, wirelessly transmit data indicative of the presence and/or quantity of one or more target analytes to a computing device 191.

According to some embodiments, as shown in FIG. 1C, a biosensor 1300 is contained in the cartridge 200. According to some embodiments, a plurality of different biomarker chemistries can be combined into one biosensor/cartridge 1300/200, thus multiple biomarker tests can be completed in parallel. According to some embodiments, specific biomarkers are combined that are related to a broader health category, for instance “Hydration”. For example, a Hydration cartridge may contain chemistry elements that can simultaneously measure sodium, potassium, and chloride.

According to some embodiments, when a test is complete (which according to some embodiments, occurs within seconds to minutes), the biofluid or saliva test cartridge 200 may be ejected and a score, e.g., a health, fitness or wellness score such as, for example, a Hydration score may be displayed on the display 110 of the Stone 100 and/or via an associated app (#1). According to some embodiments, the cartridge 200 is designed to be a disposable, single-use cartridge. According to some embodiments, underlying/related biomarker concentrations (e.g., Sodium, Potassium, Chloride measurements or score) may alternatively and/or additionally be displayed on the Stone 100 or via an app or a remote device, e.g., device 191.

Further, according to some embodiments, other biomarkers can be grouped in other ways that carry more meaningful health & fitness data to an everyday user—Energy, Fat Burn, Nutrition, Stress, etc. As an example, beyond the Hydration category, the “Energy” category might measure underlying biomarkers such as lactate, glucose and testosterone.

According to some embodiments, health categories are defined and/or additional information may be provided via the Stone 100 or app to a user based on a test result such as providing self-directed actionable insights to improve a health score. According to some embodiments, scores and biomarkers concentrations can also be shared with others—friends, family, coaches, trainer or even healthcare professionals for even more insight.

Alternatively, or additionally, beyond health & fitness, the measurement of salivary biomarkers could be used as a diagnostic screen for serious, chronic or temporary health maladies: for example, cancer, sepsis, congestive heart failure, etc.

According to some embodiments, Point-of-Care Testing (POCT) system 10 uses saliva to provide information and/or one or more scores or values related to fitness, health, and/or wellness, and/or related to a diagnostic.

According to some embodiments, the POCT system 10 determines and provides information about a person's fitness level, or general health & wellness, and/or is used to provide diagnostic information in a less expensive, invasive and/or inconvenient manner, e.g., as opposed to having to visit a physician.

Collectively the system 10 provides for an electrochemical measurement, whereby the electrical characteristics of a chemical reaction are directly related to the biomarker concentration in the biofluid.

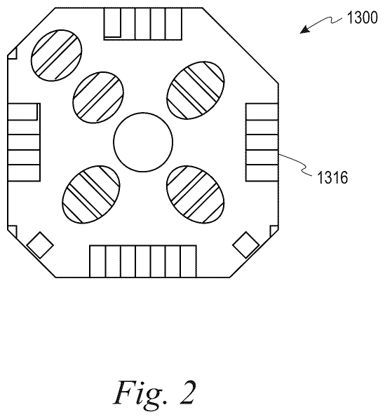

FIG. 2 illustrates a biosensor chip 1300 according to some embodiments of the present disclosure.

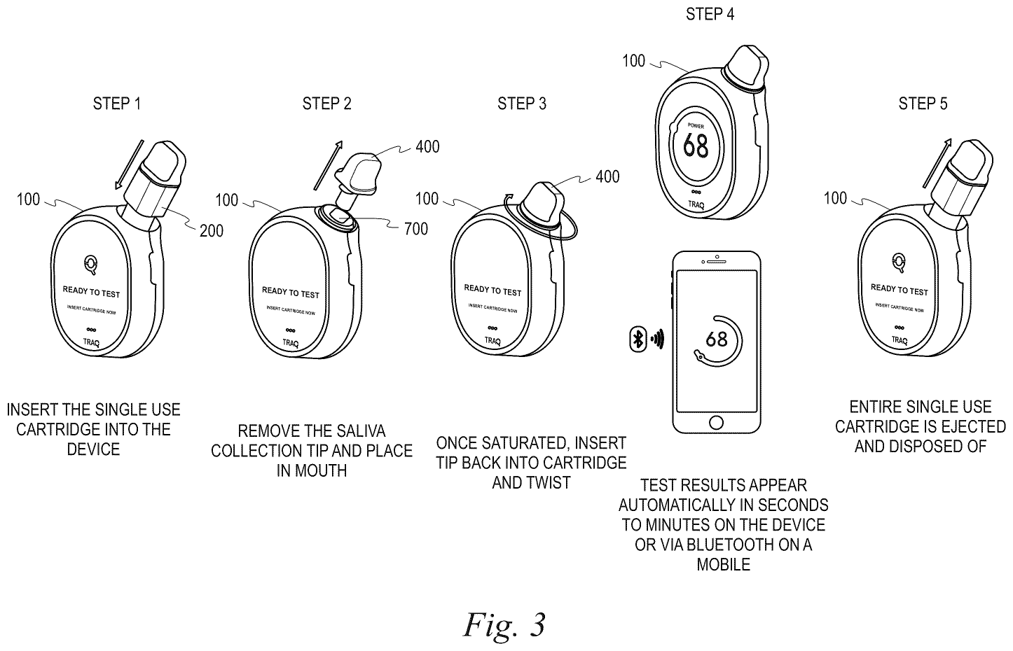

FIG. 3 illustrates an exemplary step-by-step protocol for using the POCT Stone 100 according to some embodiments of the present disclosure. According to some embodiments, in Step 1, a user would insert an entire test cartridge 200 (base 700, saliva collection tip 400) into the POCT device 100, and then (Step 2) remove the biofluid or saliva collection device 400 from the cartridge base 700 to collect a saliva sample from a mouth, e.g., his or her mouth. Once complete, the user would then re-insert the collection device 400 into the cartridge base 700 (Step 3). According to some embodiments, the system 10 automatically senses a chemical reaction between the saliva sample and the biosensor chip 1300 which starts a test. According to some embodiments, the automatic sensing functions through a constant potential being held across any two of the electrode wells. On addition of saliva, there is a resistance change on those electrodes, changing the measured current. This change can be quantified and calibrated depending on the biofluid. This change triggers a processor or microcontroller 102 to start the test process. Once a test is complete, information about the test, e.g., a test score, is displayed on a display (Step 4), e.g., on display 110 of POCT device 100 and/or a different display such as a display on a smartphone, tablet, watch, etc. In Step 5, the used cartridge 200 may be ejected or removed from the POCT device 100 so that the POCT device 100 may receive a new cartridge 200.

### 2. Exemplary Cartridges and Sample Collection Devices

The sample collection device of various embodiments is configured to collect a sample from a specimen. Sample collection devices may be configured to collect biofluids from any desired region or location, for example, the mouth, the throat, from urine, from blood, from plasma, or from saliva. One exemplary sample collection device includes a unit that collects biofluid and can be compressed to distribute the sample into an analytical device.

The sample collection device of various embodiments is sized and shaped to collect a sufficiently large sample from an appropriate location of a specimen such that it is possible, using the other devices described below, to detect the presence and/or quantity of one or more target analytes in the specimen. For example, for some target analytes, such as biomarkers contained within saliva, a collection device may be a telescoping rod with an absorbent tip, such that saliva from the parotid glands may be absorbed for optimal analyte concentration. A sample collection device for collecting fluid, such as urine, blood, plasma, or saliva, may include features for compressing an absorbent portion of the device to expel sample absorbed on the absorbent portion for analyzing the expelled sample.

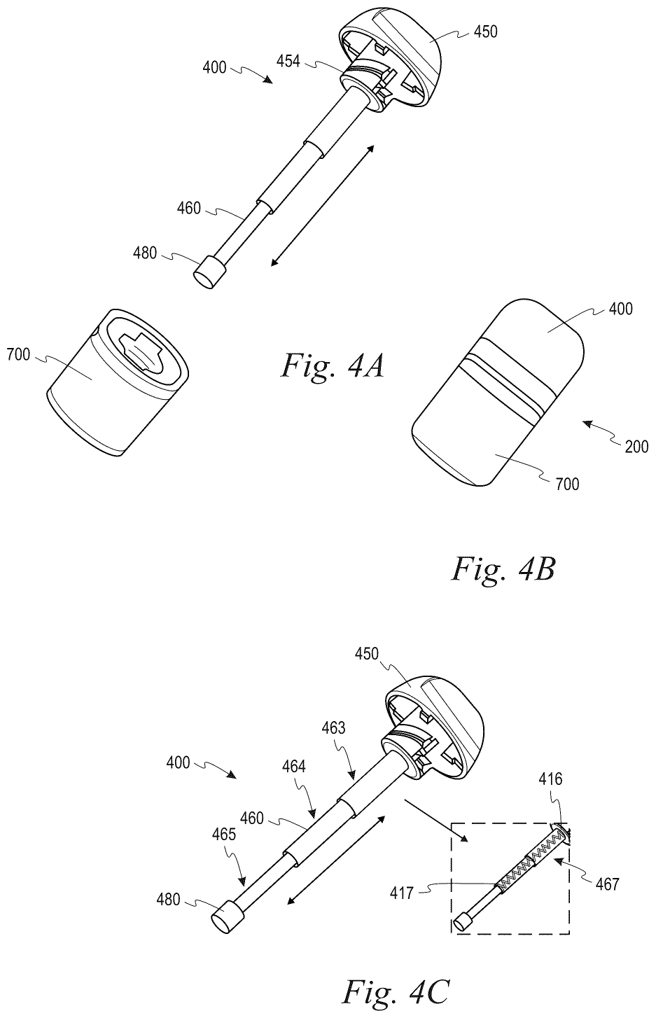

FIG. 4A is a perspective view of a disposable biofluid or saliva test cartridge 200 showing both a biofluid or saliva collection device 400 in an extended mode or position and a cartridge base 700 that serves as a collection vessel according to some embodiments of the present disclosure. According to some embodiments, the biofluid collection device 400 extends automatically upon removal from the cartridge base 700.

FIG. 4B is a perspective view the disposable biofluid or saliva test cartridge 200 with the biofluid or saliva collection device 400 in a collapsed position or mode and inserted into the cartridge base 700 according to some embodiments of the present disclosure.

FIG. 4C illustrates an example biofluid or saliva collection device 400, with an enlarged view of an interior portion of a telescoping arm according to some embodiments of the present disclosure.

According to some embodiments, the disposable cartridge 200 is comprised of two parts: a cartridge base 700 that holds an electrochemical biosensor 1300 and a biofluid or saliva collection device 400. To perform a test, a user inserts a cartridge 200 into the Stone 100, then removes the saliva collection device 400 from the cartridge base 700 to collect a sample of saliva from the oral cavity of a person or other animal or mammal (e.g., the oral cavity of the user). According to some embodiments, a sample of saliva from the oral cavity of a person is collected near the parotid duct of a person. Once a sample collection is complete (which in some embodiments takes less than 5-20 seconds), the saliva collection device 400 is inserted back into the cartridge base 700. When reinserted in the cartridge base 700, a moist collection tip 480 is compressed, releasing a saliva sample into the cartridge base 700 such as into a base chamber or reservoir in the cartridge base.

FIG. 4A illustrates a cartridge collection device 400 according to some embodiments. As illustrated in FIG. 4A the cartridge collection device 400 comprises a handle 450, a connecting mechanism 454 to secure the device 400 to the cartridge base 700, an extendable arm 460 which may be in the form of a telescoping arm, and a swab tip 480. The cartridge collection device 400 serves as a biofluid, e.g., saliva, sample collector.

FIG. 4C illustrates the cartridge collection device 400 of FIG. 4A with a portion the device's telescoping arm 460 cutaway showing an internal spring or spring mechanism 467.

When the cartridge collection device 400 is removed from its storage or cartridge base 700, it may or may not automatically expand to its full length. According to some embodiments, when the cartridge collection device 400 is removed from its storage or cartridge base 700, the spring or spring mechanism 467 automatically causes the arm 460 to transition from a collapsed or shortened position to an extended position. After the arm 460 is in an extended position, using the handle 450, a user may insert the cartridge collection device 400 into an oral cavity, e.g. mouth, for saliva collection.

According to some embodiments, the biofluid collection device 400 is configured to collect a small quantity of a sample to be analyzed and configured for full or partial insertion within cartridge base 700 after sample collection. As illustrated in FIGS. 4A and 4C, the biofluid collection device 400 comprises a handle 450, an extendable arm 460 which may be in the form of a hollow telescoping arm, and a biofluid collection tip 480. FIG. 4C further illustrates the biofluid collection device 400 with a portion the telescoping arm 460 cutaway showing a telescoping arm spring or spring mechanism 467. The telescoping arm 460 is shown in the extended position.

According to some embodiments, the biofluid collection tip 480 may be secured to the end of the telescoping arm 460 using a dental grade adhesive, such as dental glue.

According to some embodiments, the biofluid collection device 400 may not include the telescoping arm 460, and instead may only include the biofluid collection tip 480.

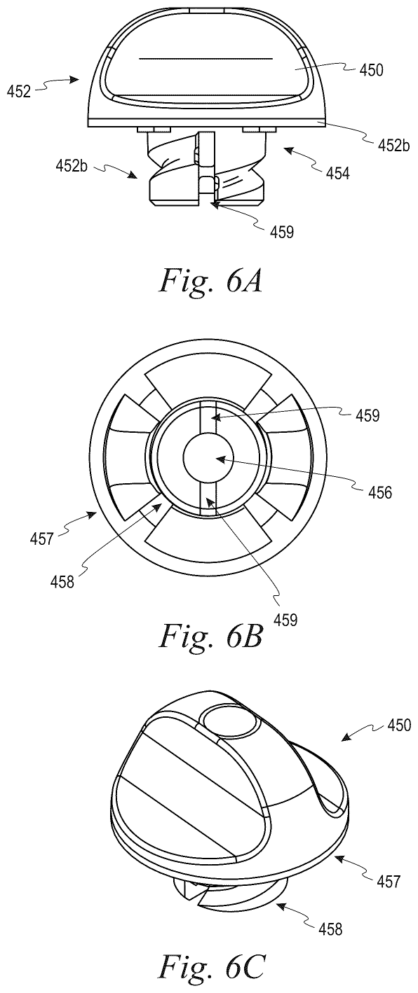

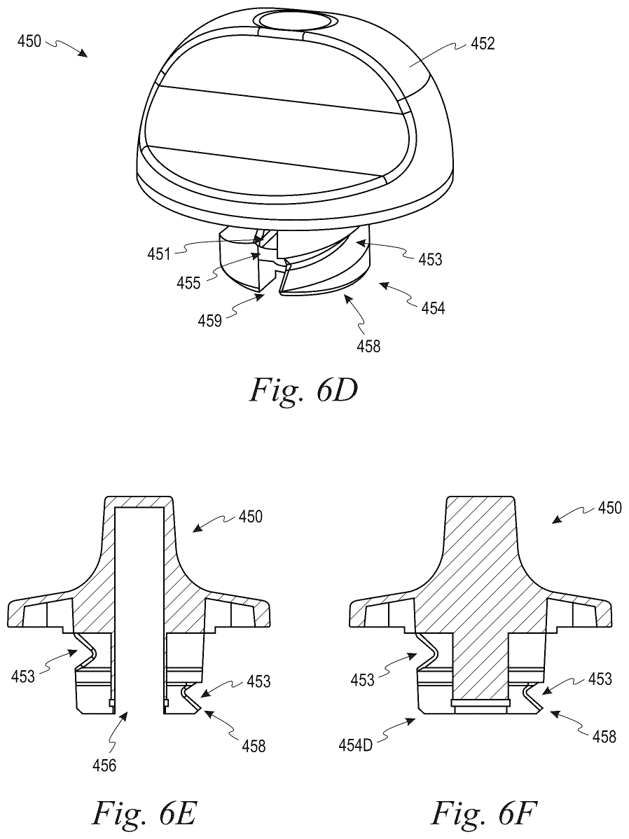

As shown in FIG. 4C, a telescoping arm 460 may comprise a spring or spring mechanism 467, a proximal joint 463, a medial joint 464, and a distal joint 465, wherein the proximal joint 463 and the medial joint 464 may be hollow cylinders and the distal joint 465 may be a solid cylinder. The spring mechanism 467 may be comprised of a proximal coil 416 and a distal coil 417. The spring mechanism 467 lies within the telescoping arm and interfaces at the proximal coil 416 with the handle 450 on a socket 456 (see FIG. 6B) of a threaded shaft 454 and is attached to the handle 450 on the socket 456. The distal coil 417 interfaces with the distal part of the telescoping arm at the distal joint 465, such that the spring mechanism 467 exerts pressure at rest on the distal joint 465, keeping the telescoping arm 460 rigid and extended. According to some embodiments, the three joints have concentric diameters, with the distal joint 465 fitting within the medial joint 464, and the medial joint 464 within the proximal joint 463, such that, when the collection device 400 is inserted into the cartridge base 700 and turned on the threaded shaft 454, the telescoping arm 460 may collapse, starting with the distal joint 465 collapsing into the medial joint 464, and then the medial joint 464 into the proximal joint 463, and then the proximal joint 463 into the socket 456 until the telescoping arm 460 is completely collapsed into the handle 450. As the telescoping arm 460 collapses into the handle 450, pressure is applied from the spring mechanism 467, which resists the torque of the handle 450 turning onto the cartridge base 700, which then compresses the collection tip 480, dispensing any biofluid held therein into the cartridge base 700.

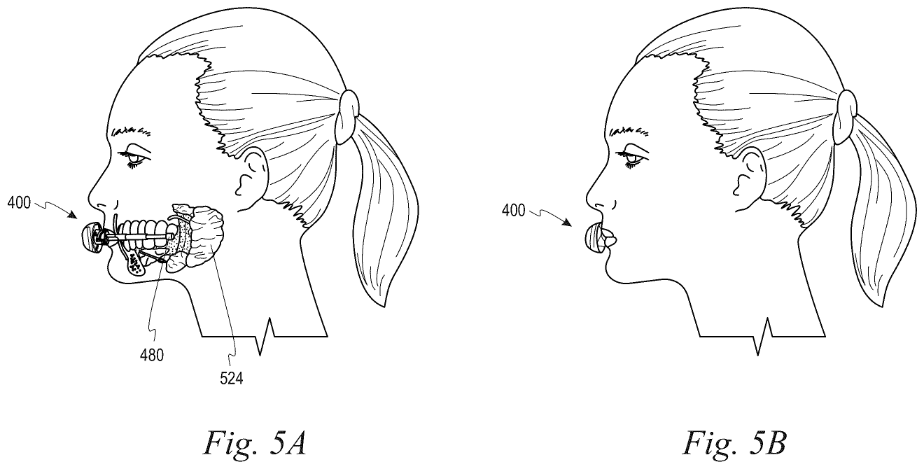

According to some embodiments, the telescoping arm 460 extends to a length that allows for sample collection directly at the parotid saliva glands 524 (see FIG. 5A) for specific sampling at the glands, rather than via pooling indiscriminately from all glands, as saliva from the parotid gland has a higher concentration of useful biomarkers and a faster response to changes in blood biomarker concentrations. The telescoping arm 460 further collapses into the cartridge base 700 during analysis, which reduces contamination that can come from the fingers of a user and allows for a smaller form factor before use.

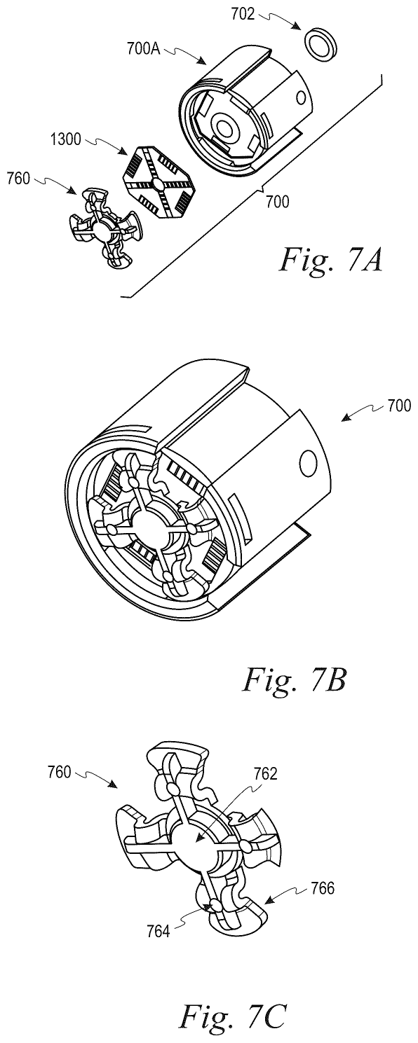

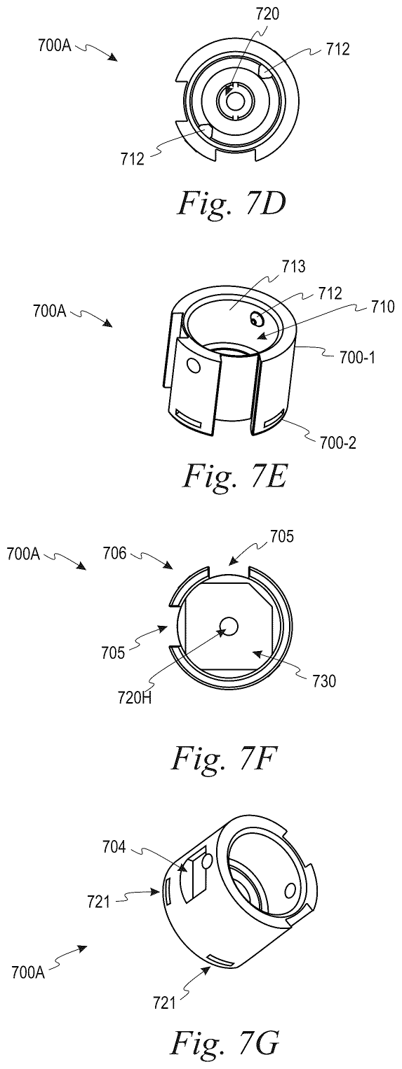

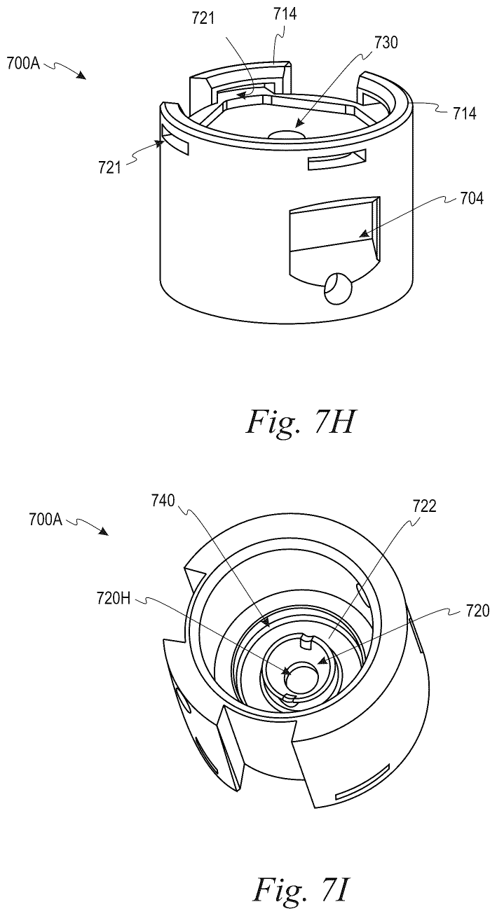

According to some embodiments, the biofluid collection tip 480 is a piece of absorbent material used to facilitate collection and storage and dispensing of the biofluid sample into the cartridge base 700 during analysis. The biofluid collection tip 480 may be sized to fit within a channel 720 (see FIG. 7I) of the cartridge base 700 such that during application of pressure when the biofluid collection device 400 is inserted into the cartridge base 700, an appropriate amount of biofluid for the analysis may be dispensed into the sensor chip 1300.

According to some embodiments, the biofluid collection tip 480 volume and absorbency is optimized to collect the exact saliva sample volume needed for analysis.

According to some embodiments, the biofluid or saliva collection tip 480 is made of some form of absorbent material, which can include, e.g., cotton, Tencel, or hemp.

According to some embodiments, some parts of the biofluid collection device 400 are made of some form of plastic, preferably non-toxic (e.g., the handle 450, telescoping arm 460).

According to some embodiments wherein a telescoping arm 460 is used, the biofluid collection device 400 is removed from the cartridge base 700 and inserted into an oral cavity, at a specific location, e.g., close to the parotid gland for saliva collection, or more generally moved around for collection, such that the handle 450 remains outside the mouth but the biofluid collection tip 480 is on or near the parotid gland 524, and the telescoping arm 460 is fully extended, as seen in FIGS. 5A-5B. Once the user has saturated saliva into the collection tip 480 for a suitable amount of time for collection, e.g., for 5-20 seconds or until the tip is fully moist, the collection device 400 may be reattached to the cartridge base 700 for analysis.

According to some embodiments wherein other biofluids are used, e.g., blood or urine, the biofluid collection device 400 is removed from the cartridge base 700 and inserted into a sample of the biofluid of note, e.g., blood or urine, such that the biofluid collection tip 480 may become moist, at which time the collection device 400 may be reattached to the cartridge base 700 for analysis.

According to some embodiments wherein a telescoping arm 460 is not used, the biofluid collection device 400 is removed from the cartridge base 700 and inserted shallowly into an oral cavity, such that the handle 450 remains outside the mouth but the biofluid collection tip 480 is near the lips of the user. Once the user has saturated saliva into the collection tip 480 for 5-20 seconds or until the tip is fully moist, the collection device 400 may be reattached to the cartridge base 700 for analysis.

The use of telescoping arm 460 may allow for sanitary testing, e.g., in the use of blood or urine as the user does not come in contact with the sample, and/or may provide more direct contact with a specified area of an oral cavity, e.g., the parotid gland, and/or may allow for less contamination from a user that may skew results, e.g., from fingers coming in contact with the collection tip 480.

FIG. 5A illustrates a person's head with a cartridge collection device 400 inserted into an oral cavity of the person and being clenched between the teeth of the person with the tip 480 of device 400 contacting near the parotid gland and duct. In this position, saliva may be collected by the swab tip 480 as it contacts the parotid gland and duct 524. Once the swab tip 480 has been held in the person's mouth for sufficient time to allow it to collect a desired amount of saliva, the device 400 is stowed in a storage or collection vessel or cartridge base 700, see, e.g., FIG. 4B.

FIG. 5B illustrates an exterior view of a person's head with a cartridge collection device or tip 400 inserted into an oral cavity of the person as shown in FIG. 5A showing a fully inserted device 400 with the handle 450 of the collection device 400 positioned outside the oral cavity so as to reduce the risk of oral fluid contamination.

The concentration of various biomarkers and proteins in saliva varies according to whether or not the saliva was collected from a specific duct/spot or from a diluted pool within the oral cavity. For example, the concentration of salivary alpha-Amylase and Secretory IgA vary depending on the location inside the mouth at which a saliva sample is collected. Other problems with saliva collection involve the mess associated with collection, over-collection or contamination, both from a user's or person's fingertips or from the saliva sample being contaminated within the oral cavity. Hence, current saliva collection means, and techniques are either diluted (pooled), indiscriminate, messy, unsafe or results in the over-collection of saliva.

Saliva collection for diagnostics and other testing is typically done by sampling saliva pooled within the mouth in the sublingual area or pooling saliva within this area and then spitting the pooled saliva into a vessel, thus diluting key biomarkers and proteins. Methods that entail spitting of saliva also tends to be messy and potentially unsafe with the collecting vessel typically being too small or narrow. Other methods are in vivo, collecting with a long swab, scraping the inner lining of the mouth or collecting from the interior of the mouth in a non-descript fashion. Some prior methods result in more saliva than necessary being typically collected. Prior saliva collection methods (both in vivo and in vitro) fail to focus on collection at a point of duct entry into the mouth. A point of duct entry into the mouth will have greater or the greatest biomarker and protein concentration. Prior saliva collection methods (both in vivo and in vitro) also tend to be messy, or over-collect sample saliva.

According to some embodiments, an extendable, or telescoping, saliva collection mechanism 400 with a handle 450 is provided. According to some embodiments, the collection device 400 allows for sample collection directly at the duct entry into the mouth for specific sampling, rather via pooling or collecting indiscriminately, contains a swab tip 480 to optimize the saliva sample volume, and collapses into the reservoir base or collection vessel 700, minimizing mess, increasing safety and reducing the likelihood of contamination.

According to some embodiments, a saliva sample collection mechanism 400 is provided that extends, allowing for compact storage yet direct or targeted saliva sampling, a focused swab collection tip 480, for optimal, consistent collection volumes, and a retraction/collapsing mechanism 460/467 for direct application and pressure to the swab tip 480 to a biosensor 1300 residing in the collection vessel or cartridge base 700. According to some embodiments, a saliva sample collection mechanism 400 is provided that reduces or minimizes mess and increases safety.

According to some embodiments, a saliva collection mechanism 400 contains 1) a handle 450, 2) a locking mechanism 454 for connection with or insertion into a collection vessel or cartridge base 700, 3) a telescoping/collapsing arm 460 (with an internal spring design), and 4) an absorbent swab collection tip 480.

Other embodiments may include different handle designs or handles designed to be used without human touch. Further, the means of attachment 454 to a collection vessel or cartridge base 700 may not be included in an embodiment or may use an alternate attachment design, such as magnetic, suction, form fitting, threaded screw, etc. Other embodiments may include different means for extending the telescoping arm, may not be comprised of more than one section, and/or may not use an internal spring design. Further, other embodiments may employ alternate designs, shapes and materials than the swab tip 480 described above and/or shown in the accompanying figures.

Other embodiments may use a similar device 400 for the collection of other biofluids (blood, urine, sweat, tears, etc.).

Other embodiments may not be focused on human saliva or other biofluid collection, but rather may be directed to animals (pets, livestock) saliva or other biofluid collection and the length or structure of the device 400 may be different for such embodiments.

According to some embodiments, a collection device 400 may be provided having one or more or all of the following features:
a telescoping collection arm 460 that focuses collection where it is best-suited, reduces likelihood of contamination, and/or increases safety;
a collection device closure with associated collection vessel or base 700 that facilitates the safe handling of a biofluid sample, while simultaneously expressing or releasing saliva from the swab tip 480 to an appropriate location within the base 700 such as to a biosensor.
an optimized swab tip size to help minimize over-collection that leads to safety and contamination issues, as well as mess. According to some embodiments, the swab tip 480 is made of some form of absorbent material, which can include, e.g., cotton, Tencel, or hemp. The swab tip 480 may be optimized to contain the optimal saliva sample volume, in some embodiments the size of the tip may have a diameter of 3-7 mm, and may have a length of 5-7 mm, or be of such shape to accommodate an acceptable saliva sample, of which the optimal saliva sample size may range from 1-10 uL in some embodiments and the size of the tip 480 may be configured to collect such a volume of saliva. Further, other embodiments may employ alternate designs, shapes and materials than the swab tip 480 described above and/or shown in the accompanying figures.

According to some embodiments, some parts of the device 400 and/or cartridge base are made of some form of plastic, preferably non-toxic (e.g., the handle 450, collection vessel mechanism 454, telescoping arm 460, collection vessel or cartridge base 700). According to some embodiments, the swab tip 480 is made of some form of absorbent material, which can include, e.g., cotton, Tencel, or hemp.

According to some embodiments, the saliva collector 400 as a whole is envisioned to be integrated with the collection vessel or cartridge base 700. The collector 400 and the cartridge base 700 together form a saliva collection mechanism for the system 10. According to some embodiments, the length of the collection vessel or base 700 is preferred to be shorter than the length of the telescoping arm 460, to the point where the swab tip 480 is compressed against an interior surface of the base 700, such as against a biosensor, when the collection mechanism 400 is inserted within and/or secured to the collection vessel or base 700.

According to some embodiments, using the handle 450, the saliva collector 400 is removed from the collection vessel or cartridge base 700 (e.g., via snap, magnetic, twist, etc.), whereby the saliva collector 400 extends to its full length upon removal.

According to some embodiments, the collection mechanism 400 is then inserted into an oral cavity, at a specific location, e.g., close to the parotid glad for saliva collection, or more generally moved around for collection. Once the user has saturated saliva into the collection tip 480 for 5-20 seconds or until the tip is fully moist, the collection mechanism 400 is reattached to the collection vessel or cartridge base 700. The collection vessel 700 may or may not be part of a larger saliva testing device 100 or system 10.

According to some embodiments, one or more or all of the following advantages may be achieved using the collection device 400 and/or cartridge base 700:

Directed saliva collection—Research has shown that direct collection of saliva from specific salivary ducts (either parotid gland or submandibular/sublingual gland) results in higher concentrations of salivary biomarkers or proteins (e.g. RNA, DNA, etc.). Relatively, pooled saliva typically holds lower concentrations. Having higher concentrations of an interested biomarker or protein within a sample, increases the ability to make an accurate measurement and/or reduces the chances that follow-on or additional sampling and testing will be needed to obtain an accurate test result.

Less contamination, more safety—less or restricted interaction of the fingertips with the saliva sample means that the sample is less likely to be contaminated. Further, the likelihood of transmission from the saliva to fingertips is also less likely.

Less mess—Some current collection mechanisms rely on a pooled sample that is then spit into a collection vessel. Aside from possible contamination, this leads to spillage and increased mess.

Optimized collection volume and high concentration of target molecules—using the optimized swab tip, only the amount required to be collected, is indeed collected. Comparing to other current means, there is an over-collection, which leads to mess, contamination and safety issues.

FIG. 3, Step 2 illustrates the removal of a biofluid or saliva collection device 400 from an inserted cartridge base 700 according to some embodiments of the present disclosure. FIG. 5A illustrates the collection of a biofluid such as saliva according to some embodiments of the present disclosure. FIG. 3, Step 3 illustrates the reinsertion of a biofluid or saliva collection device 400 into an inserted cartridge base 700 according to some embodiments of the present disclosure.

According to some embodiments, after a disposable biofluid or saliva test kit or cartridge 200 is inserted in the reader device 100, just the biofluid collection device 400 is removed (FIG. 3, Step 2), the user collects a saliva sample (FIG. 5A) and the sample collection device 400 with a now moistened tip is then reinserted into the cartridge base 700. FIG. 3, Step 2 illustrates the removal of a biofluid collection device 400 from a cartridge base 700 positioned in the reader device 100 where the biofluid collection device 400 is twisted off from the cartridge base 700. Once the collection device 400 has been removed from the cartridge base 700, a user may collect a saliva sample using the collection tip 480. The insertion of the collection tip 480 of the collection device in a cavity of a user or subject from who or which a biofluid is to be retrieved is shown in FIG. 5A. In FIG. 3, Step 3, the collection device 400 is reinserted into the cartridge base 700.

### 3. Cartridge

According to some embodiments, a disposable sensing unit or cartridge 200 for use with the TRAQ Stone 100 consists of a biosensor chip 1300 which is integrated into a modular cartridge 200 which is comprised of a collecting vessel/reservoir base 700 and an integrated saliva collection device 400 (e.g., handle 450, telescoping cotton swab for saliva collection). The biosensor chip 1300 resides in the base 700 of the disposable cartridge 200.

A. Cartridge Handle Portion 450

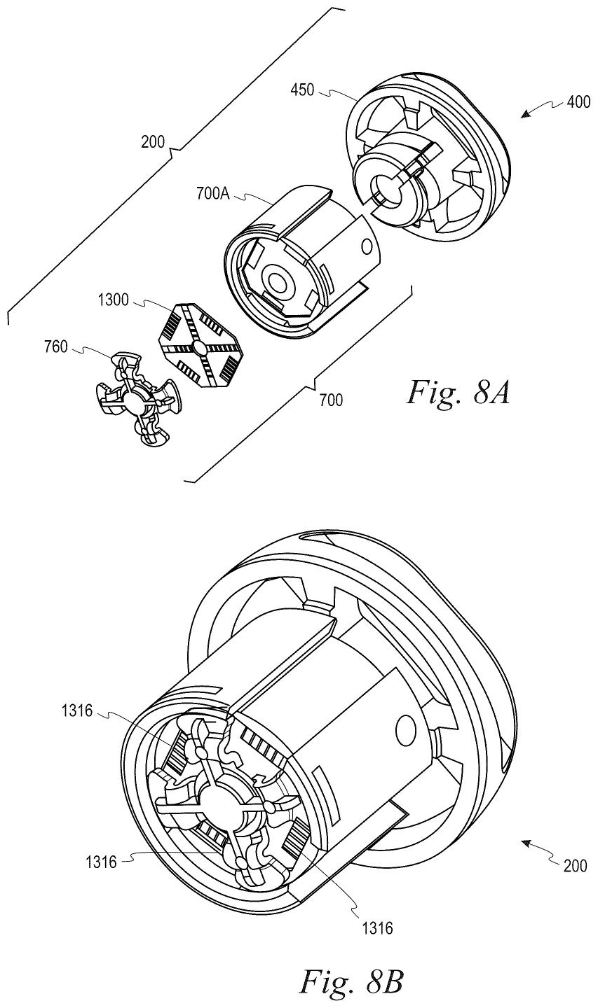

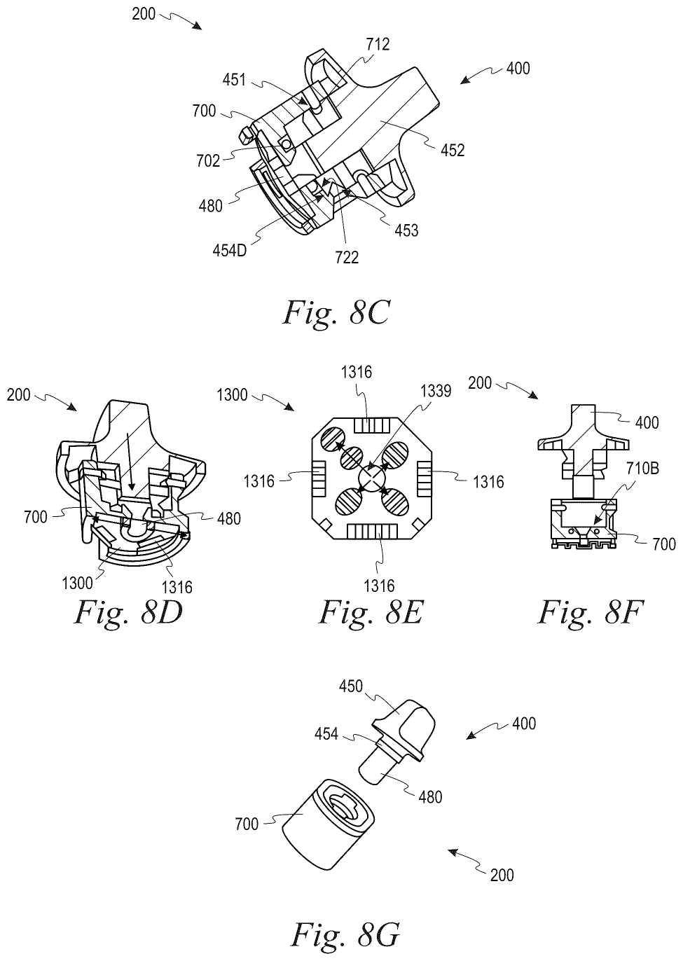

FIG. 6A is a side view, FIG. 6B is a bottom view, FIG. 6C is a perspective view, and FIG. 6D is an enlarged view of handle 450, and FIG. 6E and FIG. 6F are cross sectional views of two embodiments of handle 450 of a biofluid collection device 400. The handle 450 may comprise a curved handle knob 452 and a threaded shaft 454 as seen in FIG. 6A. The threaded shaft 454 may constitute an extrusion from a base 452b of the handle knob 452, which may have a diameter complimentary to that of an insertion chamber 710 of the cartridge base 700 such that the threaded shaft 454 may lock the biofluid collection device 400 to the cartridge base 700 with the collection tip 480 located within the insertion chamber of the cartridge base 700. As seen in FIG. 6D, the threaded shaft 454 may further comprise one or more vertical grooves 459 formed within the threaded shaft 454 such as on each side of the threaded shaft 454 exactly or generally opposite to one another to assist with securing the biofluid collection device 400 to the cartridge base 700, such that when the collection device 400 is pushed into the cartridge base 700, one or more protrusions 712 on an interior wall 713 of the insertion chamber 710 of the cartridge base 700 may aligned with and inserted into the vertical grooves 459 until the protrusions 712 meet or engage with a lip 455, whereby when a handle knob 452 is twisted the protrusions 712 on an interior wall of the insertion chamber 710 of the cartridge base 700 may travel within a female thread 453 located on the shaft 454, allowing for the collection device 400 to then be twisted using the handle knob 452 to apply torque until the protrusions 712 of the cartridge base 700 lock into a top groove 451 formed in the shaft 454. Biofluid collection device 400 may also include a proximal sealing zone 457 seen in FIG. 6B configured for sealing the insertion chamber 710 of cartridge device 700 when the collection tip 480 of the biofluid collection device 400 is inserted in the insertion chamber 710 and the collection device 400 is twisted into engagement with the cartridge base 700. According to some embodiments, the proximal sealing zone 457 may be formed from a protrusion extending around the threaded shaft 454 and sized greater than the opening of the insertion chamber 710 to seal the insertion chamber and any biofluid from the collection tip 480 therein. As such, the protrusion(s) 712 may further lock the distal end of the biofluid collection device 400 within the insertion chamber 710 of cartridge base 700. According to some embodiments, the threaded shaft 454 may further comprise a distal sealing zone 458 formed near a distal end 454D of the threaded shaft 454 which may press onto, abut, and/or engage with a bottom surface 722 of the insertion chamber 710 of the cartridge base 700 and that, when coupled with an O-ring 702 in the cartridge base 700 (see FIG. 8C), may prevent backflow of biofluid into the chamber 710 during dispensing biofluid from the collection tip 480. The threaded shaft 454 may further comprise a handle socket or chamber 456 open on one side, e.g., a distal or a bottom side, of the shaft 454 as seen in FIG. 6B. According to some embodiments, a proximal end of a telescoping arm 460 is secured to the handle 450 near or within the handle socket 456. According to some embodiments, the handle socket 456 may extend into the handle 450 a sufficient distance to allow for the telescoping arm 460 to fully compress into the handle 450 as seen in FIG. 6E. In other embodiments, in which a telescoping arm 460 may not be used, the socket 456 may be shallow and not extend into the handle 450 as seen in FIG. 6F.

According to some embodiments, the telescoping arm 460 may be secured to the handle 450 in the socket 456 using a dental grade adhesive, such as dental glue.

According to some embodiments, the biofluid collection device 400 has the advantage of not having any electronics and/or a battery and/or other power source therein. According to some embodiments, the biofluid collection device 400 has the advantage of not having any electronics and/or a battery and/or other power source on the portion thereof configured to be received in a cavity of a body in which a biosample is to be collected using the biofluid collection device 400 such as an oral cavity in which saliva is to be collected. For example, according to some embodiments, the biofluid collection device 400 has the advantage of not having any electronics and/or a battery and/or other power source on the extendable arm 460 and/or the collection tip 480.

B. Cartridge Base

The collection tip 480 of the collection device 400 may be locked into or be seated into the cartridge base 700 to eliminate or reduce any chance of oral fluid leakage. According to some embodiments, the collecting device 400 may lock into and/or be seated into the cartridge base 700 to reduce or eliminate the chance of oral fluid leakage from the chamber 710 of the cartridge 200. According to some embodiments, the device 400 and the base 700 engage and seal each other to reduce the chance of oral fluid leakage out of the cartridge 200.

The torque applied from the twisting motion of the collection device 400 into the cartridge base 700 allows for more force to be applied to the collection tip 480 of the collection device 400 with less effort than if a simple insertion method were used.

FIG. 7A is an exploded perspective view and FIG. 7B is an assembled perspective view of the cartridge base 700 according to some embodiments. The cartridge base 700 may comprise an O-ring 702, cartridge base body 700A, a latch 760, and a sensor chip 1300. The cartridge base 700 is configured to house the biofluid collection device 400 before use, receive the biofluid collection device 400 with saturated collection tip 480 during analysis, and electrically interface with the reader device 100 during analysis. Based on the analysis, cartridge device 700 is configured to generate electric signals indicative of the presence and/or quantity of one or more target analytes within the sample.

According to some embodiments, the cartridge base body 700A is made via injection molding but can also be 3D printed (e.g., ABS, TPU, PLA, etc.). According to some embodiments, the cartridge base body 700A comprises of three major regions: a collection device insertion chamber 710, a saliva reservoir 720, and a sensor chip cavity 730. According to some embodiments, the collection device insertion chamber 710 has a cross-sectional circular shape, with two extruded small protrusions 712 on opposite sides near a first end 700-1, e.g., top, of the cartridge base body 700A. The cartridge base body 700A also has a second end 700-2, e.g., bottom.

FIG. 7C is a perspective view of a latch 760 for the cartridge base according to some embodiments of the present disclosure.

FIG. 7D is a top view, FIG. 7E is a side perspective view, FIG. 7F is a bottom view, FIG. 7G is another side perspective view, FIG. 7H is an upside-down, side perspective view, and FIG. 7I is downward perspective view showing an inner view of a chamber 710 of a cartridge body 700A of a cartridge base 700 according to some embodiments of the present disclosure. The cartridge body 700A may comprise a collection device insertion chamber 710, a lever retaining groove 704, one or more cutout grooves 705 such as two cutout grooves 705, an island connector 706, one or more latch grooves 721 such as four latch grooves 721, and a sensor chip cavity 730. The collection device insertion chamber 710 may comprise a channel 720 and having a hole 720H formed therein, an O-ring groove 740, and one or more protrusions or thread pins 712 such as two protrusions 712 which protrude from an interior wall 713 of the insertion chamber 710. The hole 720H in the channel 720 serves as an aperture for biofluid to flow from the insertion chamber 710 to a chip 1300 during biofluid analysis. The channel 720 leads to the underside of the cartridge body 700A, wherein according to some embodiments the hole 720H matches in size and/or location to a similar hole on one side, e.g., a bottom side, of the sensor chip 1300, such that, when the biofluid collection device 400 is inserted into the cartridge base 700 and the pressure on the collection tip 480 causes biofluid to be dispensed from the collection tip 480 into channel 720, the biofluid may flow to the sensor chip 1300 for analysis. According to some embodiments, the channel 720 and the O-ring groove 740 are concentric grooves or formations in in one end 710B, e.g., a bottom end, of the insertion chamber 710 (see FIG. 8F, 7I), such that the O-ring groove 740 surrounds the channel 720. The O-ring groove 740 may be sized and configured to house the O-ring 702, which may be used to seal the interface between the collection tip 480 and the channel 720 when biofluid is being dispensed to prevent backflow from the channel 720. For example, as the biofluid collection device 400 is inserted and twisted into the cartridge base 700, the collection tip 480 is compressed against the collection channel 720 between the distal sealing zone 458 of the handle 450 and the channel 720 and O-ring 702. The O-ring 702 deforms under the pressure to seal the interface. According to some embodiments, the swab 480 is compressed when the handle 450 of the saliva collection device 400 is twisted and locked into the base 700, facilitating saliva to flow onto the biosensor chip 1300 housed therein. According to some embodiments, the locking aspect also seals the cartridge 200, preventing leakage, for safety reasons.

According to some embodiments, the protrusions 712 are configured and positioned to engage with the threads 453 of the collection device 400 allowing for a screwing motion and facilitating a tight seal (see, e.g., FIG. 8C). According to some embodiments, the protrusions 712 may travel through the vertical grooves 459 of the handle 450 on the threaded shaft 454 and slide into the threads 453 on the threaded shaft 454, such that, when the collection device 400 is inserted into the insertion chamber 710, the protrusions 712 may be inserted into the vertical grooves 459 until reaching the lip 455 of the vertical grooves 459, at which point the biofluid collection device 400 may be twisted and the protrusions 712 slide into the threads 453 of the threaded shaft 454, until locking into the top groove 451 of the collection device 400.

According to some embodiments, a cartridge body 700A may further comprise a lever groove 704, which may allow a release lever on the reader device 100 to interface with the cartridge base 700, such that when the cartridge base 700 is inserted into a cartridge receptable 140 of the reader unit 100, the release lever may insert into the lever groove 704 and immobilize and/or hold the cartridge base 700 in the cartridge receptacle 140 of the reader unit 100 from longitudinal forces for analysis.

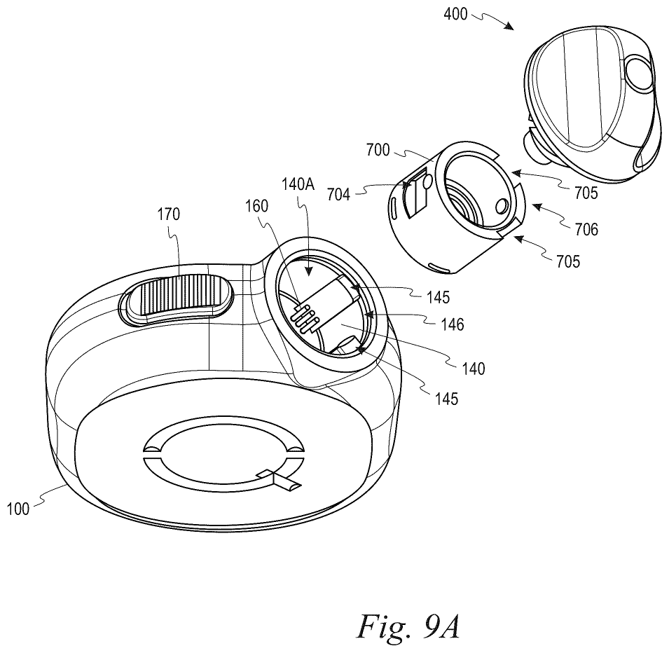

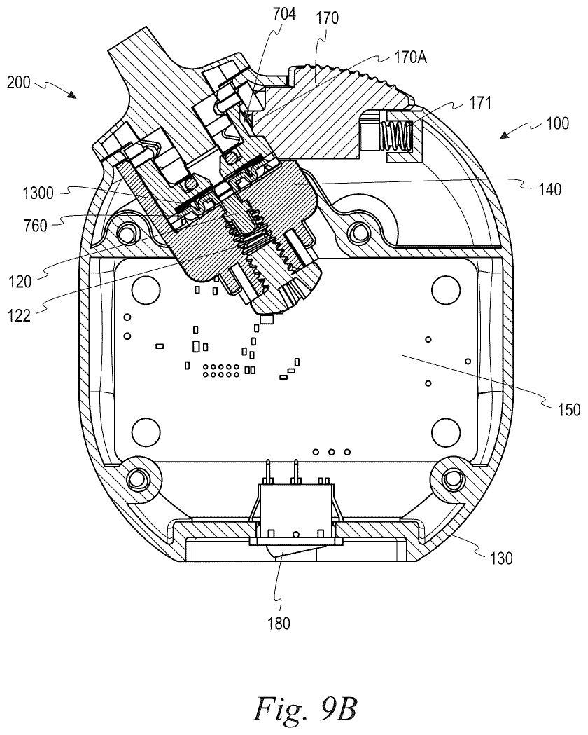

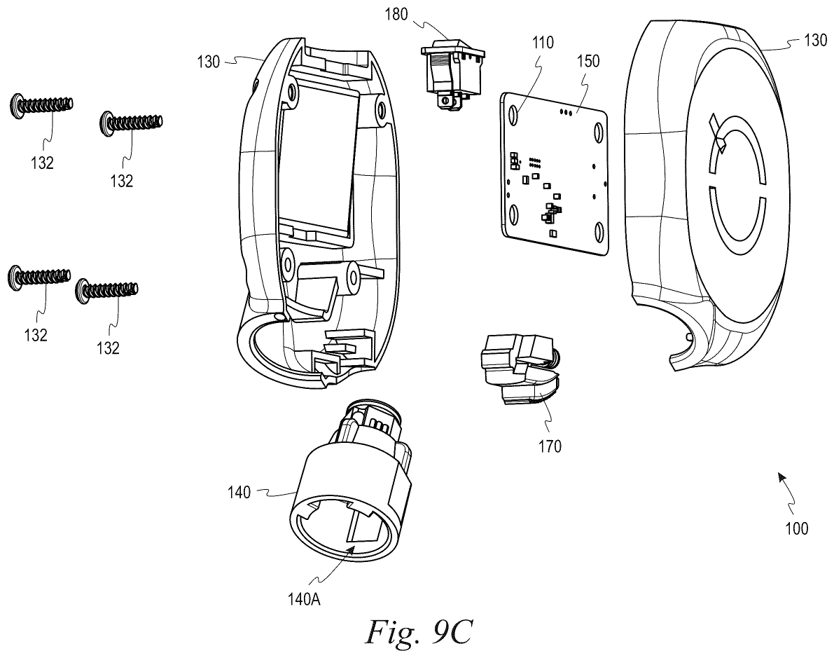

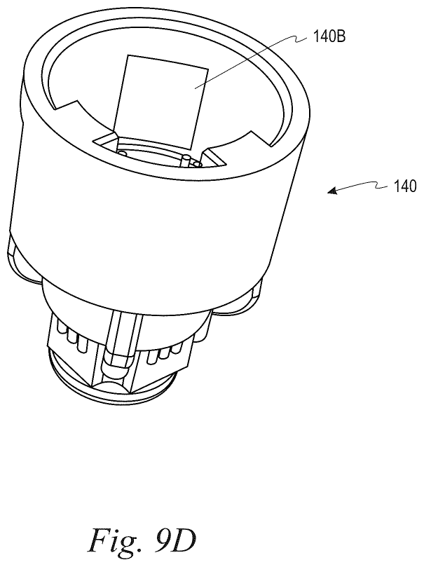

According to some embodiments, a cartridge body 700A may further comprise a pair of cutout grooves 705 and an island connector 706 (see, e.g., FIGS. 7F and 9A) which lay of the outside of the cartridge body and may be sized and positioned to mate with connector mechanisms having projecting cartridge electrical connectors 145 and matching groove 146 associated with the cartridge receptacle 140/140A of the reader device 100, such that, when a cartridge base 700 is inserted into a cartridge receptable 140 of the reader unit 100, the cutout grooves 705 of the cartridge 700 are aligned with the pair of projecting cartridge electrical connectors 145 in the cartridge receptacle 140/140A. Likewise, the island connector 706 is aligned with a cutout groove 146 in the cartridge receptacle 140. As the cartridge 700 is inserted into the cartridge receptacle 140/140A, the island connector 706 may slide into and be received in the cutout groove 146 in the cartridge receptacle 140/140A and the protecting cartridge electrical connectors 145 may be received in the cutout grooves 705 of the cartridge 700, immobilizing and/or holding and/or locking the cartridge base 700 in the cartridge receptacle 140 against rotational forces so the handle 452 of the collection device 400 may be turned or screwed into and screw out of the cartridge base 700 while the cartridge base 700 is secured in the receptacle 140A during use and/or sample analysis.

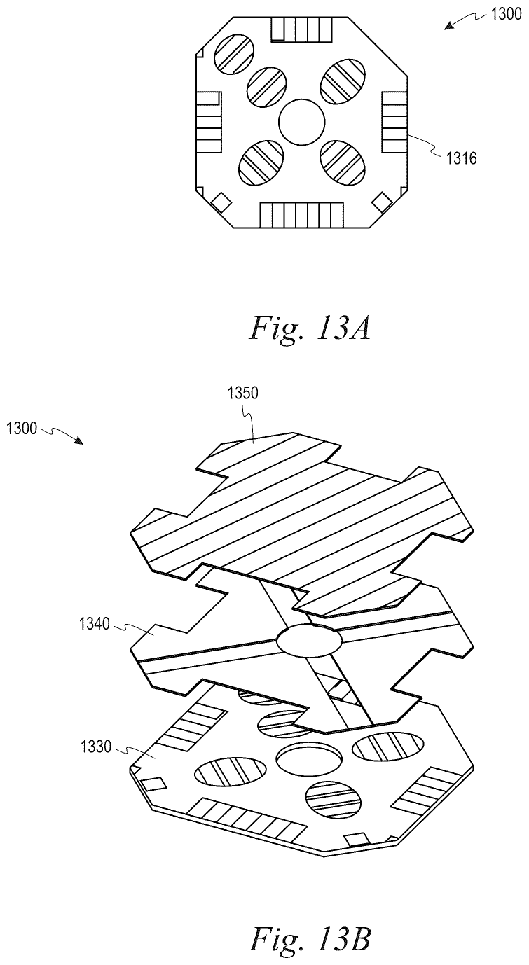

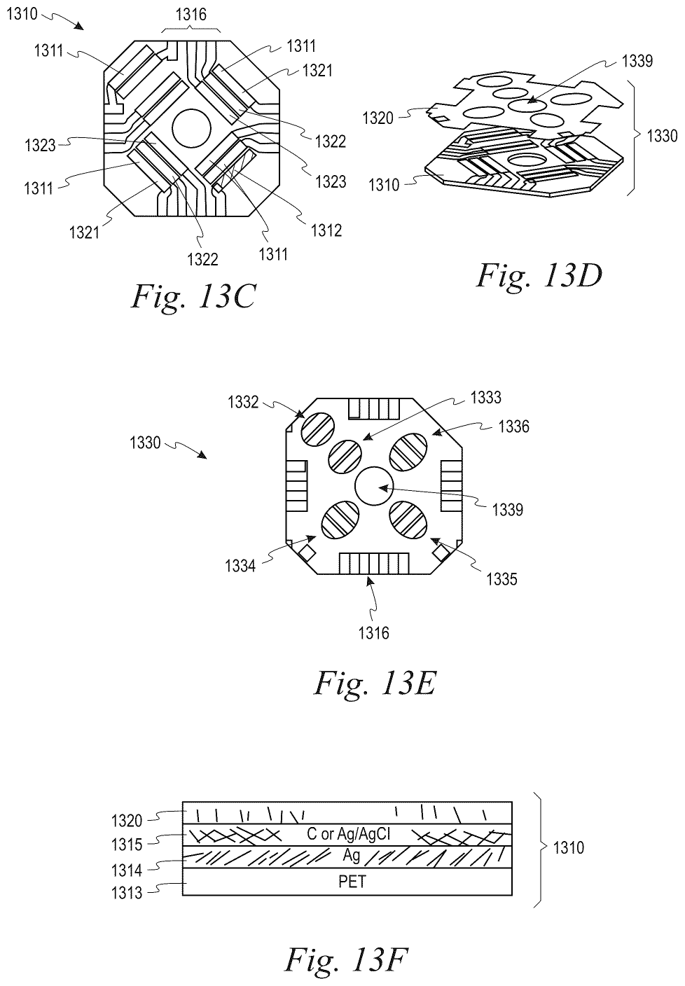

According to some embodiments, a cartridge body 700A may further comprise a sensor chip cavity 730 which is located on one side, e.g., the underside, of the cartridge body 700A and which may be shaped and sized to accommodate and position a biosensor chip 1300 therein. According to some embodiments, when a biosensor chip 1300 is placed in the sensor chip cavity 730, it is immobilized and/or held therein. According to some embodiments, when a sensor chip is positioned within the sensor chip cavity 730, the hole 720H of the channel 720 may be aligned with a center hole 1339 (see FIGS. 8E and 13E) of the sensor chip 1300.

According to some embodiments, a cartridge body 700A may further comprise one or more latch grooves 721 such as four latch grooves 721. According to some embodiments, the latch grooves 721 are formed in one or more latch engagement protrusions or flanges 714 located on one side, e.g., an underside, of the cartridge body 700A surrounding the sensor chip cavity 730, and which may be set in pairs across from each other forming a cross, and that may interface with the feet 766 of the latch 760, such that, when a biosensor chip 1300 is placed in the sensor chip cavity 730, each foot of a latch 760 may be inserted into a corresponding latch groove 721, so that the latch 760 can be placed adjacent the chip 1300 to secure the biosensor chip 1300 in the sensor chip cavity 730.

According to some embodiments, a sensor chip 1300 faces DOWN relative to the cartridge base portion 710A, so that the saliva flows down through the hole 720H and through a bottom sensor chip hole 1339 onto the hydrophilic cover 1320, which spreads the saliva along to the electrodes 1311, 1312, 1321, 1322, 1323.

According to some embodiments, a latch 760 may comprise a hub 762, one or more elbow joints 764 such as four elbow joints 764, and one or more feet 766 such as four feet 766, as seen in FIG. 7C. According to some embodiments, the feet 766 of the latch 760 extend further than the diameter of the cartridge body 700A and/or of sufficient distance so as to be permitted to be securely inserted in the latch grooves 721 and engages with the latch engagement protrusions or flanges 714. A latch 760 may be used to further secure the biosensor chip 1300 when it is placed in the sensor chip cavity 730.

According to some embodiments, the latch 760 may further comprise one or more, e.g., four, elbow joints 764, which radiate outwards from a hub 762 and couple the feet 766 of the latch 760 to the hub 762. According to some embodiments, an elbow joint 764 may be formed from a flat piece of material that is compressed into an elbow or bent shape which allows the elbow joint 764 to compress towards or away from the hub 762 in the radial axis, such that, when a latch 760 is inserted adjacent a biosensor chip 1300 in the sensor chip cavity 730, each foot 766 can be inserted into a corresponding latch groove 721 when the corresponding elbow joint 764 of the foot 766 is compressed radially inward, and once the pressure on the elbow joint 764 is released and the foot 766 is inserted into the latch groove 721, the elbow joint 764 can extend to its resting position and lock the latch 760 on the cartridge body 700A.

C. Cartridge Handle and Base

After manufacturing, the cartridge base 700 and collection device 400 are stored together in a sterile packaging, wherein the collection device 400 is partially compressed into the cartridge base 700, and this complete assembly may be referred to as a biofluid test kit or cartridge 200.

FIG. 8A is an exploded perspective view and FIG. 8B is an assembled perspective view of a subassembly of a completed portion of a disposable biofluid test kit or cartridge 200 with a handle 450 of collection device 400, cartridge body 700A, sensor chip 1300 and latch 760. In FIG. 8A, the extendable member 460 and absorbent tip 480 have been omitted from the view of the collection device 400. FIG. 8B shows how the cartridge 200 subassembly is assembled and packaged before use.

FIGS. 8C-8D are assembled perspective cross-sectional views and FIG. 8F is an unassembled side, cross-sectional view of a disposable biofluid or saliva test cartridge 200 according to some embodiments. FIG. 8E is a top view of the biosensor chip 1300 according to some embodiments. FIG. 8C shows the cartridge 200 with the biofluid or saliva collection device 400 collapsed or compressed and shows how the handle 452 is locked to the cartridge base 700 via the protrusions 712 on the cartridge base 700 resting in the top groove 451 formed in the threaded shaft 454 (see also FIGS. 4C, 6A, 6D). FIG. 8D shows the biofluid or saliva collection device 400 compressed into the cartridge base 700 whereby the biofluid may be expelled from the absorbent tip 480 and onto the biosensor chip 1300, wherein the arrows show the flow of fluid from the tip 480 onto the biosensor chip 1300. FIG. 8E further shows just the biosensor chip 1300 and an exemplary flow of a sample fluid therein when the tip 480 is compressed against channel 720 and a collected bio-sample flows through hole 720H in the channel 720 and through a center hole 1339 (see FIGS. 13D and 13E) of chip 1300 according to some embodiments.

FIG. 8F shows the biofluid or saliva test cartridge 200 with the collection device 400 separated from the cartridge base 700.

FIG. 8G is a perspective view of a cartridge 200 containing a collection device 400 that does not employ a telescoping arm according to some embodiments of the present disclosure. Instead, an absorbent tip 480 is coupled directly to the shaft 454 of a handle 450.

D. Cartridge Summary

According to some embodiments, the cartridge base 700 not only houses the biosensors 1300, but also serves as storage for the saliva collection device 400, a reservoir for the saliva sample but also contains electrical connections 1316 of the biosensor 1300 to connect the cartridge 200 with the POCT device 100 such as via electrical contacts 160 located in a cartridge receptacle 140.

According to some embodiments, the cartridge 200 and the cartridge base 700 have the advantage of not having a battery and/or other power source therein.

### 4. Reader Device

FIG. 9A is an exploded perspective view of a collection device 400, a cartridge base 700, and a reader 100 which may take the form of a handheld POCT testing device according to some embodiments of the present disclosure.

FIG. 9B is a cross-sectional view of a reader/POCT testing device 100 with a test cartridge 200 inserted in a cartridge receptacle 140 (e.g., as would be during use) according to some embodiments of the present disclosure. The cartridge receptacle 140 has a cartridge cavity 140A therein for receiving a cartridge base 700 of a cartridge 200.

FIG. 9C is an exploded view of the reader/testing device 100 and some of its component parts according to some embodiments of the present disclosure. The cartridge receptacle 140 serves as a receptacle for a biofluid or saliva test cartridge 200.

FIG. 9D is a perspective view of a cartridge receptacle 140 having an aperture 140B therein according to some embodiments of the present disclosure.

The reader 100 may comprise a release lever 170 and a power switch 180 according to some embodiments.

According to some embodiments, the reader 100 is a handheld point-of-care testing device that contains the computational power (e.g., a CPU or processor), electrical capabilities and wireless, e.g., Bluetooth, communication capabilities. According to some embodiments, the reader 100 comprises a cartridge receptacle 140, an analysis computer, CPU(s), or processor(s) located on one or more printed circuit boards (“PCB”) 150, and a power source such as a battery (not shown).

According to some embodiments, the cartridge receptacle 140 may also comprise a cutout or aperture 140B (see FIG. 9D) through which a release lever 170 may pass so as to come into contact and/or abut a cartridge 200 residing in the cartridge receptacle 140. For example, when a cartridge 200 is inserted in the cartridge receptacle 140, a portion of the release lever 170, e.g., a cartridge engagement protrusion 170A, may pass through the cartridge receptacle aperture 140B and into the retaining lever groove 704 of the cartridge base 700 to thereby hold the cartridge 200 in the cartridge receptacle 140. The reader 100 may also comprise an internal lever spring 171 for application of a biasing pressure to bias the lever 170 into engagement with a cartridge 200 located within the cartridge receptacle 140 to hold the cartridge base 700 in the cartridge receptacle 140.

According to some embodiments, the cartridge receptacle 140 itself may be made of plastic. According to some embodiments, an interior surface of cartridge receptacle 140 defining the cartridge cavity 140A of the cartridge receptacle 140 has a complimentary geometry to the exterior of the cartridge base 700 to aid in properly positioning and/or holding a cartridge base 700 within the cartridge receptable 140.

According to some embodiments, the cartridge receptacle 140 may contain reader electrical contacts 160 to facilitate electrical and/or communication coupling between the cartridge base 700 and/or the biosensor chip 1300 located therein (e.g., via electrical contacts 1316) and the reader 100. According to some embodiments, the reader electrical contacts 160 are spring loaded (or magnetic) contacts. According to some embodiments, the reader electrical contacts 160 are positioned within the cartridge cavity 140A such as on or near the bottom of the cartridge receptacle 140 and are positioned to contact or couple with corresponding cartridge electrical contacts located on the cartridge base 700 (such as cartridge electrical contacts or pads located on or near the bottom of a cartridge base 700. According to some embodiments, the cartridge electrical contacts or pads are located on the sensor chip 1300 located within a cartridge base 700.

According to some embodiments, the reader 100 may comprise communication hardware and may be configured to be communicatively coupled to one or more external devices 191 such as a smartphone, watch, tablet, and/or computer so as to permit communication (e.g., send and/or receive data) to/from such external device(s). According to some embodiments, the reader 100 comprises Bluetooth hardware, such as, e.g., Bluetooth Low Energy (BLE) and is configured to communicate with an external device via a Bluetooth protocol. According to some embodiments, the reader 100 sends data out to a phone (e.g., smartphone) through Bluetooth.

According to some embodiments, the reader 100 may comprise a display 110 and be configured to display information on the display 110 instead of and/or in addition to sending to and/or facilitating the display of information on a remote device 191.

According to some embodiments, the cartridge base 700, saliva collection device 400, and swab 480 are stored together in the closed position. When a user uses the device 100, they place the cartridge 200 into the Stone 100 and press it in until it clicks with the latch 170, and is flush with the surface of the receptacle 140. Once the cartridge 200 is seated and latched in the reader receptacle 140, a user may then twist the collection device 400 off the cartridge base 700. A user can then saturate the swab 480 with a biofluid, e.g., saliva, and replace the collection device 400 into the cartridge base 700 located in the receptable 140 of the reader 100. During replacement of the collection device 400 into the base 700, a twisting motion of the collection device 400 will force saliva down into contact with the sensor 1300 for analysis to begin.

According to some embodiments, the hub 762 (see FIG. 7C) may be in the center of the latch 760 and be configured to interface with a button 120 (see FIG. 9B) located in the cartridge receptacle 140, such that, when a cartridge base 700 is inserted into a cartridge receptable 140 of the reader unit 100, the button 120 in the cartridge receptacle 140 of the reader 100 may press against the hub 762 which places pressure on the biosensor chip 1300 so that it remains secure and aligned with the hole 720H in the channel 720 of the cartridge base 700 and sample fluid may flow into the biosensor chip 1300. The pressure exerted by the button 120 in the cartridge receptacle 140 of the reader 100 on the hub 762 may also force the cartridge base 700 into the release lever 170 on the reader device 100. For example, when a cartridge base 700 is inserted into the cartridge receptable 140 of the reader unit 100 and the release lever 170 of the reader unit 100 is inserted in the lever groove 704, the pressure, e.g., upward pressure, of the button 120 of a cartridge receptable 140 of the reader unit 100 may act in concert with an opposing pressure, e.g., downward pressure, applied by the release lever 170 of the reader 100 to hold and/or immobilize the cartridge base 700 within the cartridge receptable 140 of the reader unit 100. According to some embodiments, the button 120 is biased into the cavity 140A of the cartridge receptacle 140 by a biasing member 122 such as a spring.

With reference to FIG. 9C, according to some embodiments, the reader 100 may comprises a housing formed of one or more housing pieces 130. According to some embodiments, multiple housing pieces may be secured to each other via one or more fastening means such as screws 132.

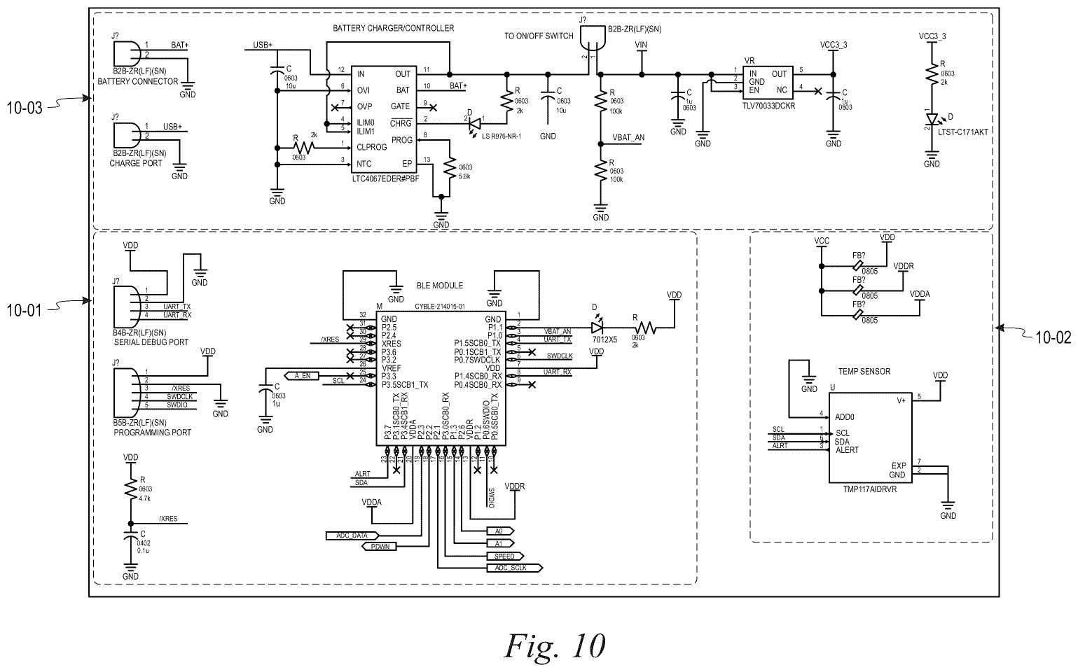

FIGS. 10-12 are plan views of PCB schematics according to some embodiments of the present disclosure. According to some embodiments, various analysis methods described herein (e.g., enzymatic and ISE) may have their own corresponding PCBs, which are integrated to function together within the reader/stone 100. According to some embodiments, the multiple PCBs shown in FIGS. 10-12 may be combined into one PCB for stability.

In FIG. 10, circuit 10-01 illustrates a system on chip microcontroller circuit according to some embodiments. Circuit 10-02 is a temperature sensor that can be used to correct the measurements for hydration if there is a temperature dependence. Circuit 10-03 is a battery management system that handles the charging of a Li-ion battery and disperses power to the device.

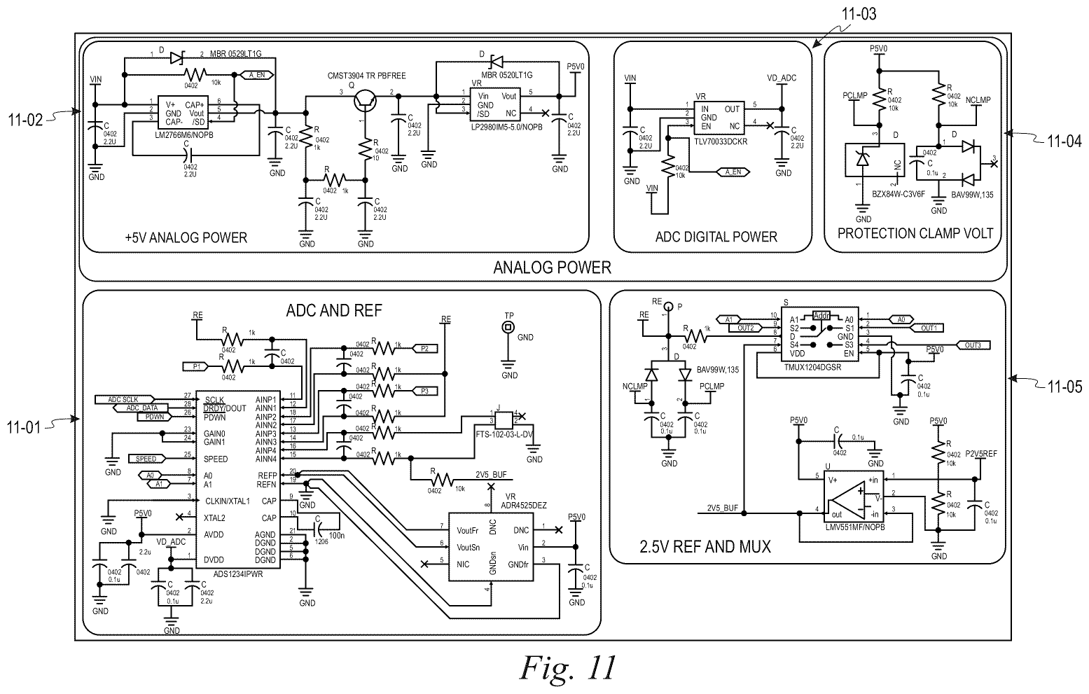

In FIG. 11, circuits 12-01 are potentiostat channels. As shown, there are three channels that can be monitored and/or recorded at once. These channels may correspond to different wells.

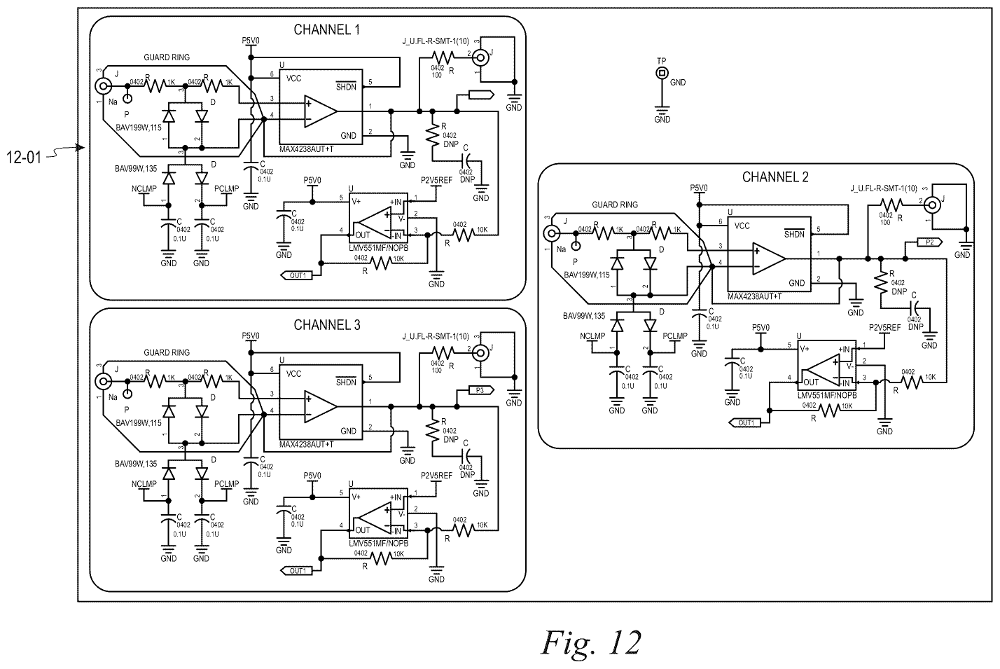

In FIG. 12, circuit 11-02 may be employed to create clean 5V power from variable battery voltage. Circuit 11-04 may be used to create a pair of clamp voltages for ESD protection. Circuit 11-05 mutliplexes the analog output of the per channel signal conditioning into the ADC. Circuit 11-01 is an ADC and Reference that convert analog voltages from the potentiostat into digital signals to be read in the microcontroller 10-01.

According to some embodiments, the ISE PCB 150 (see FIG. 9C) is a mixed signal board that handles the potentiometry/voltammetry, analysis, uplink and power handling for the reader 100. According to some embodiments, the digital components include the serial debug/programming ports, a temperature sensor to detect overheat, a power switch, and a battery charger/controller along with the connectors and charge port. According to some embodiments, the digital domain also includes the BLE module which is configured to communicate with an external device 191 such as a user's smartphone. This BLE module is contained within a microcontroller, e.g., a CYBLE-214015-01 module (although other microcontrollers could be used). According to some embodiments, the analog components include the 5V analog power, digital power and protection clamp volt. According to some embodiments, analog components may also include a Reference voltage chip and a multiplexer, which allows the signals from the different channels being measured to come together. According to some embodiments, an ADC is also used to convert analog sensor information to a digital signal usable by the microcontroller.

According to some embodiments, three data points are obtained in the beginning, middle and near the end of an amperometric analysis when a biosensor 1300 is configured to detect the presence and/or quantity of an analyte enzymatically or through immunoassay. A calculated concentration of an analyte is determined by a processor 102 such as by employing calibration curves.

According to some embodiments in which the biosensor chip 1300 is configured to detect the presence and/or quantity of an ionic analyte, periodic voltage measurements are taken, which are then converted to a concentration of ion by the Nernst Equation in the CPU of the reader/Stone 100, without any calibration steps needed.

### 5. Biosensor Chip

According to some embodiments, a cartridge base 700 further comprises a sensor chip (e.g., biosensor) chip 1300, which can be configured for analyzing a sample and interfacing with the reader 100 to determine the presence and/or quantity of one or more target analytes within the sample.

FIG. 13A is a top view of a biosensor chip 1300 according to some embodiments. The chip 1300 comprises a plurality of electrical contacts 1316.

FIG. 13B shows an exploded perspective view of a biosensor chip 1300, which may comprise various layers. According to some embodiments, the chip 1300 comprises a sensor chip base 1330, spacer layer 1340, and a top cover layer 1350 according to some embodiments of the present disclosure, and a biosensor chip 1300 may also comprise a sensing film atop the printed layer, not shown here.

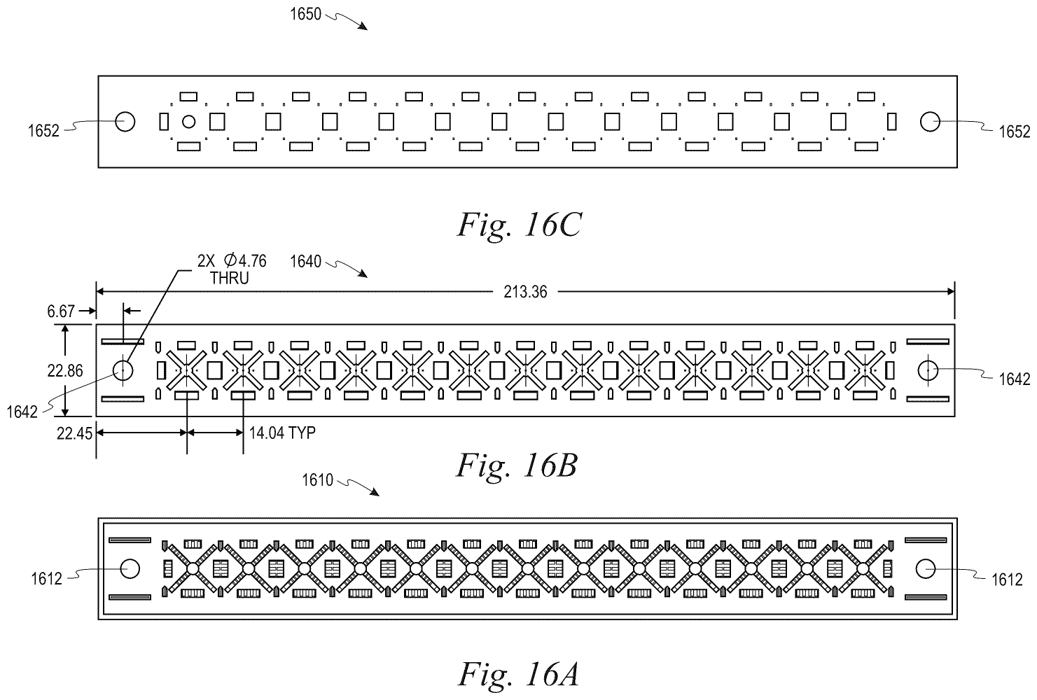

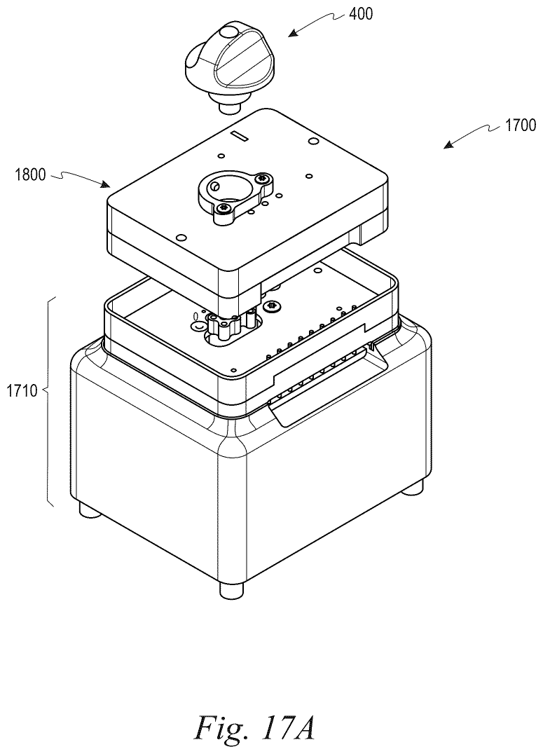

In FIG. 13B a sensor chip 1300 is shown having a single spacer layer 1340 and a single top cover layer 1350 added to the sensor chip base 1330 according to some embodiments of the present disclosure. According to some embodiments, during manufacturing, the layers are added in a strip design to make a plurality of sensors (see, e.g., FIGS. 16A-16C) and not individually as shown here.

FIG. 13C shows a top view of screen-printed electrode layers 1310 of a biosensor chip 1300 according to some embodiments of the present disclosure. FIG. 13C shows the screen-printed pad layers 1310 (Ag, carbon or another material) according to some embodiments of the present disclosure.

FIG. 13D is an exploded perspective view of the screen-printed electrode layers 1310 and a dielectric layer 1320 of a biosensor chip 1300 according to some embodiments of the present disclosure. The screen-printed electrode layers 1310 and the dielectric layer 1320 are referred to collectively as a sensor chip base 1330. FIG. 13D shows a hydrophobic dielectric layer 1320 added on top of the screen-printed electrode layers 1310 according to some embodiments of the present disclosure.

FIG. 13E is a top or plan view of a sensor chip base 1330, shown in FIG. 13D according to some embodiments of the present disclosure.

Referring to FIG. 13C, the printed electrode layers 1310 may comprise a plurality of electrical contacts or pads 1311, 1312. Referring to FIGS. 13E and 13C, the pads 1311 may be grouped within different wells 1332, 1333, 1334, 1335, and 1336. These wells are referred to generally as wells 1331. According to some embodiments, a three pad well 1336 and a three pad well 1334 are made up of three carbon pads 1311, and a distal well 1332 and a proximal well 1333 are made up of two carbon pads 1311. Another three pad well 1335 comprises two carbon pads 1311 and one Ag/AgCl pad 1312.

According to some embodiments, the pads 1311, 1312 in each well 1331, and each well 1331 generally, have different uses depending on which configuration the biosensor chip 1300 is manufactured for, allowing for a modular design when designing new types of biosensor chips 1300 within this existing framework for determining the presence and/or quantity of an analyte, wherein the wells 1331 used or the sensing films that are deposited within said wells 1331 can be changed to fit a certain configuration. Some sample configurations for biosensor chips 1300 will be discussed. In some embodiments, all 3 pads in a well 1334, or well 1336 or both pads in a well 1332 or well 1333 can be used for the same purpose, in which case the well 1331 will be referred to by its use. For example, instead of one pad serving as a “working electrode”, if all 3 pads in a well 1334 or well 1336 are used together as working electrodes then the well will be referenced as the “working well”.

According to some embodiments, the electrical contacts 1316 and the wells 1331 are formed by a screen-printing, or the like, methodology, wherein multiple screen-printed electric and dielectric layers are printed on a substrate 1313.

Referring to FIG. 13C, the carbon pads, shown without hatching, are grouped together with similar pads such that when the dielectric layer 1320 is placed over the printed layer 1310, the wells 1331 that are formed may function as single measurement units. In this case, a well 1331 may comprise a complete electrochemical measurement system, such as, but not limited to, a three-electrode system wherein there may be a working electrode, a reference electrode, and a counter electrode.

FIG. 13E shows a top view of the sensor chip base 1330, which may comprise a hole 1339, electrical contacts 1316, and (for example) five wells 1331, which include a distal two pad well 1332, a proximal two pad well 1333, and, continuing in a clockwise fashion from the proximal two pad well 1333, a three pad well 1336, a three pad well 1335, and a three pad well 1334. According to some embodiments, a hole 1339 may match a hole 720H of a cartridge base 700, such that, when the biosensor chip 1300 is placed in the sensor chip cavity 730, the hole 720H permits a fluid expelled from an absorbent tip 480 to pass into the middle of the sensor chip cavity 730 and eventually through the hole 1339 of the biosensor chip. Thus, according to some embodiments, a biofluid sample has an unobstructed flow path from the channel 720 onto the hydrophilic top cover 1350 of a biosensor chip 1300. The hydrophilic top cover 1350 facilitates spreading the biofluid along to the wells 1331 for analysis therein.

FIG. 13F is a side schematic view illustrating various screen-printed layers of a sensor chip base 1330 according to some embodiments of the present disclosure. According to some embodiments the sensor chip base 1330 is printed with a Ag (silver) first layer 1314, a carbon second layer 1315 which makes up the electrode pads 1311 (with Ag/AgCl forming one electrode or pad 1312 shown with hatching in FIG. 13C), and dielectric third layer 1320. In some embodiments, the Ag layer acts as a conductor throughout the chip 1300 that electrically couples the pads 1311, 1312 to the outside of the chip 1300. According to some embodiments, the carbon is printed on top of a PET substrate 1313, or any other suitable material to serve as a substrate, as the traces or pads for measurement. The dielectric layer 1320 placed over the screen-printed electrode layers 1310 has apertures therein that define the wells 1331 over the pads 1311, 1312 for biofluid sample analysis to occur therein. The dielectric layer 1320 serves as a hydrophobic barrier to aid in pushing or directing fluid towards the wells 1331 in conjunction with the hydrophilic top cover 1350 and the spacer 1340. When the dielectric layer 1320 is printed on top of the other two layers 1315, 1314, the Ag traces 1314 are covered, leaving only the exposed edge regions as the electrical contacts 1316. Referring to FIG. 13F, according to some embodiments, the biosensor chip 1300 is made up of multiple screen-printed electric and dielectric layers on a PET substrate 1313, although any suitable flexible but stable polymer could work as well according to some embodiments.

According to some embodiments, the Ag layer 1314 acts as a conductor throughout the chip 1300 and wires electrically connect the contacts 1316 (which make electrical contact with electrical contacts 160 inside the Stone 100) to the enzymatic/ion selective electrodes (ISE) 1311, 1312. According to some embodiments, the carbon or Ag is printed on top as the contact pads 1316 for the electrodes 1311, 1312 themselves.

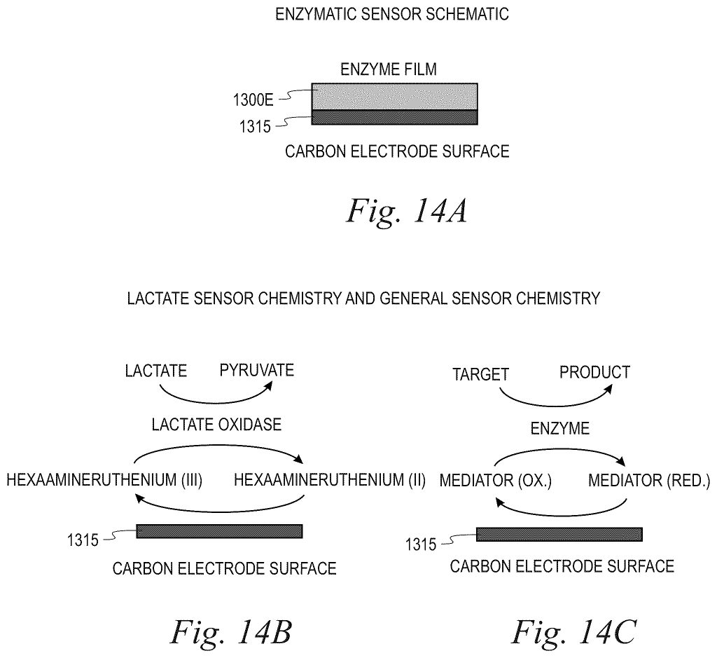

FIG. 14A is a layer stackup of the enzymatic sensing film 1300E for a lactate sensing film, or an enzymatic sensing film generally. The film 1300E may be drop-cast as one layer into the wells 1331 (see FIG. 13E), wherein the film coats the carbon pads 1311, 1315 (see FIGS. 13C-13F) of chip 1300.

According to some embodiments, a biosensor chip 1300 may be configured to determine the presence and/or quantity of an analyte using an enzymatic reaction, wherein a working electrode counts the electrons added to a well 1331 by a mediator during a redox reaction. Specifically, according to such embodiments, chronoamperometry is used between a reference electrode and a working electrode. According to some such embodiments, the three electrode wells 1334 and 1336 are used for determining the presence and/or quantity of an analyte, and contain a sensing film 1300E (e.g., as shown in FIG. 14A), and the remaining wells are not used. According to some embodiments, the measurements determining the presence and/or quantity of an analyte between both wells are averaged for better accuracy. In such embodiments, one pad 1321 in each well 1334, 1136 (e.g., the outermost pad) acts as a sample fill detector pad, which has a small constant current running through it, such that, when a sample is dispended into the cartridge base 700 and further into the biosensor chip 1300, the resistance may change on the sample fill detector pad 1321 which changes the current. According to some embodiments, when circuitry (e.g., a processor 102) in the reader 100 detects such a change in current, the processor causes a set of instructions (e.g., stored in memory 106) to run on the reader 100 (e.g., a processor 102 therein) that causes measurement to begin as to signals associates with the other pads in the wells 1334, 1336. The other two pads 1322 and 1323 act as a working electrode and a reference electrode, respectively, for the associated well 1334, 1336.

According to some embodiments, accuracy is determined using blood or saliva samples measured by commercial grade machines for the same sample as used on the sensors, devices and systems described herein. Accordingly, comparisons between results obtained using the devices, sensors, and systems described herein and commercial grade machines may be made and results may be calibrated.

According to some embodiments, the chip 1300 comprises one or more saliva wells 1331, one or more reference electrodes 1323, one or more electrical contacts 1316, and one or more working electrodes 1322. According to some embodiments, the biosensor chip 1300 consists of a screen-printed working electrode 1322 with physical depressions or wells 1331 which contain enzymatic chemistry for detecting specific biomarkers in saliva, along with electrical connection points or contacts 1316, for interaction with a measuring tool or device 100.

According to some embodiments, when a saliva sample comes into contact with the biosensor 1300 in the cartridge base 700, an electrochemical test may start automatically. According to some embodiments, each biosensor 1300 contains both electrical and chemistry elements. According to some embodiments, the broader chemistry aspects can be broken down into three classes of reactions: enzymatic, ion-selective, and an immunoassay class. According to some embodiments, immunoassay testing may require a different form factor of the reader 100 and/or cartridge base 700 (which will be described in more detail below).

According to some embodiments, the sensor chip 1300 faces “down” relative to the cartridge base portion 700A, so that the saliva flows down through the reservoir hole 720H and through a bottom sensor chip hole 1339 onto a hydrophilic cover 1350, which aids in spreading the saliva along to electrodes 1311, 1312.

According to some embodiments, different classes of chemical tests present in chips 1300, either in part or in full, are differentiated by the addition of a small RFID tag to the top of chip 1300, which sends data such as the type of sensor, sensor ID, test(s) ID, types of tests on the sensor, model and/or serial number of the sensor and/or calibration data to the processor 102 of the reader unit 100. In some embodiments, the differentiation is not done by RFID but instead done through the mobile app, wherein such information about a cartridge to be inserted or inserted in a cartridge receptacle of the reader 100 is manually selected on a remote device such as a smartphone and then communicated to the processor 102 in the reader unit. In some embodiments, the differentiation is performed by physical “lock and key” mechanisms whereby a cartridge 200 with certain physical or mechanical shapes or protrusions (a “key”) fits into corresponding physical receiving shape (the “lock”) in the cartridge receptacle 140. Electrical contacts on the cartridge 200 and cartridge receptable 140 could then electrically transmit a message to the processor 102 indicating a lock and key fit for various cartridge test types. According to some embodiments, the chip 1300 may have a memory therein and such information (e.g., information about the type of sensor and/or the tests contained therein) is stored in the memory and communicated to the processor 102 of the reader unit 100 when the corresponding cartridge base 700 is received in the cartridge receptacle 140.

A. Exemplary Types of Measurements

According to some embodiments, chemically, the biosensors 1300 may contain chemicals related to measuring the concentration in any of a variety of chemical reactions such as the following three, or more, chemical reaction classes: Enzymatic, Ion Selective, or Immunoassay.

ENZYMATIC:

DETECTION METHOD: Amperometry

According to some embodiments, an enzymatic sensor 1300 works by measuring the electrons added to a working electrode by a redox mediator, e.g., Hexamine rhuthenium (II), during a redox reaction. Specifically, chronoamperometry is used between various electrodes. According to some embodiments, multiple current data readings, e.g., three data points, are averaged in the beginning, middle and near the end of the downslope of the analysis and related measurements and averaging may be performed by one or more processors 102 in the reader 100 communicatively coupled to a sensor 1300. According to some such embodiments, an average current value is converted to potential and then a concentration of an analyte is determined by a processor 102 using a standard calibration curve. For lactate, the potential (voltage) used is 100 to 300 mv. Any potential (voltage) may be used as long as it achieves reduction potential range for that specific biomarker.

ION SELECTIVE:

DETECTION METHOD: Open circuit potentiometry

According to some embodiments, an Ion Selective (ISE) sensor 1300 uses open circuit potentiometry (OCP) to detect ion analytes in a biosample such as saliva. According to some embodiments, there are two electrode types used for the measurements: a screen-printed Ag/AgCl reference electrode 1312, and a screen-printed carbon electrode 1311. According to some embodiments, the electrodes 1311 collect electrons and one or more potentiostats 104 compare the voltage (potential) between the electrodes 1311 and the reference electrode 1312, with a current kept constant between them. A processor 102 converts the voltage measurements to measures of concentrations of ions by the Nernst Equation, without any calibration steps needed.

IMMUNOASSAY:

DETECTION METHOD: Amperometry

According to some embodiments, chronoamperometry on an unmodified screen-printed carbon electrode 1800-E detects an amount of reacted tetramethylbenzidine (TMB). For TMB, the potential (voltage) used is 100 to 300 mv. Any potential (voltage) may be used as long as it achieves reduction potential range for that specific substrate. The detected amount is inversely proportional to the amount of cortisol (or other analytes) present in the saliva. According to some embodiments, a processor 102 makes a standard curve using known cortisol concentrations in a buffer which can then be used by the processor 102 to correlate current with cortisol concentration. According to some embodiments, the processor uses a 4-PL curve fit (Cortisol competitive immunoassay), but other parameterized logistic curves could be used. See below for more information.

Regardless of the chemical reaction class, according to some embodiments, electronics in the reader/POCT device 100 measure electrical characteristics of the chemical reaction(s) on the biosensor 1300 within a cartridge 200. The relationship between the electrical characteristics of the chemical reaction and, e.g., lactate concentration is a known quantity. Thus, according to some embodiments, firmware and/or software in the reader/POCT device 100 converts the electrical characteristics to a biomarker concentration when the reaction is complete (e.g., within 30 seconds to 12 minutes).

B. Enzymatic and/or Ion Selective Sensors

In embodiments wherein a biosensor chip 1300 is configured to determine the presence and/or quantity of an analyte using an enzymatic reaction, a sensing film 1300E according to some embodiments can be described as a second-generation biosensor, in which a redox mediator is used to transfer electrons from the enzymatic reaction to an electrode surface to reduce dependency on oxygen mediation for better signal, shown in FIGS. 14B and 14C. FIGS. 14B-14C show an embodiment of a biosensor chip 1300 for measuring lactate and a general schematic of the reactions used according to some embodiments. According to some such embodiments, a sensing film 1300E comprises a reagent preferably comprising an enzyme, a redox mediator, at least one binder, and a surfactant. According to some embodiments, the enzymes can be lactate oxidase (LOx) for lactate detection, glucose oxidase or dehydrogenase for glucose, or lactose dehydrogenase for pyruvate. Some embodiments of a biosensor configured to determine the presence and/or quantity of an analyte using an enzymatic reaction includes lactate as the target analyte and lactate oxidase as the redox mediator. In general, any analyte that can be detected enzymatically for which there exists suitable chemistry can be measured, such as glucose, cholesterol, uric acid, alcohol, ketones, triglycerides, etc. According to some embodiments, the enzymes used are from the class of oxidase, dehydrogenase, reductase, or esterase. These enzymes could include glucose dehydrogenase (GDH), glutamate dehydrogenase, glucose oxidase, cholesterol oxidase, cholesterol esterase, lactate oxidase, ascorbic acid oxidase, alcohol oxidase, alcohol dehydrogenase, bilirubin oxidase, and the like.

According to some embodiments, the redox mediator is a small redox active molecule that reacts with enzyme active sites and an electrode active surface 1315. The mediator is reduced by the ensuing electrons from the enzyme primary reaction, and then oxidized at a working electrode surface, whereby it transfers an electron to the working electrode and produces a current signal proportional to the detected analyte concentration. The mediator may comprise a transition metal complex in which one or more ligands are coordinated to the transition metal and a polymer backbone such as one or more kinds selected from the group consisting of poly(vinylpyridine) (PVP) or poly(vinylimidazole) (PVI), and poly allyl glycidyl ether (PAGE), and selectively, a linker structure connecting the polymer backbone and transition metal complex. The transition metals used could include Os, Rh, Ru, Ir, Fe and Co and the ligands could be monodentate, bidentate, tridentate or quadridentate. These will usually contain nitrogen and be heterocyclic (ammine, pyridine, imidazole derivative). For example, hexaammine ruthenium(III), ferricyanide, ferrocene, pentaaminechlororuthenium(III) chloride, etc. According to some such embodiments, the redox mediator used is hexaammineruthenium(III) (hereinafter to referred to as “HR3”) chloride because of its structural and chemical reversibility between the (II) and (III). HR3 is converted from the (III) to (II) and back after gaining or losing an electron respectively, so it is perfect for single electron reactions such as that of LOx.

According to some embodiments, a binder may comprise an inorganic polymer and/or an organic polymer. In some such embodiments, the inorganic polymer is PVP ([poly]vinylpyridine), and the organic polymers are trehalose and Methocel. In general, a binder can comprise inorganic polymers (PVC, PVP, etc.) as well as organic (trehalose, Methocel, cellulose, etc.). According to some embodiments, the use of trehalose, Methocel, and PVP as the binder for a sensing film 1300E allows for a greater than 12-month shelf life and higher stability.

According to some embodiments, a surfactant helps spread the sensing film, which includes the enzyme, mediator and polymer backbone, evenly across an electrode. According to some embodiments, the surfactant used is Triton X-100. Other surfactants could be used such as sodium dodecyl sulfade, perfluorooctane sulfonate, and sodium stearate.

According to some embodiments, a sensing film 1300E for a biosensor chip 1300 configured to detect the quantity of lactate in a sample may be fabricated in the following manner. HEPES and BTP are combined in aqueous solvent to make a buffer solution. NaCl and TritonX are similarly dissolved in aqueous solution to make respective stock solutions. PVP and hydroxyethyl cellulose are centrifuged and vortexed in an aqueous solution to form a polymer solution. Trehalose and sodium succinate are added to the polymer solution and stirred. The surfactant (TritonX, NaCl) solutions are added and the polymer solution is centrifuged and vortexed. Buffer solutions are added with Methocel and stirred for at least 1 hour to disperse in solution. Ruthenium hexamine is added to an aliquot of the polymer solution and centrifuged/vortexed and stirred for an hour. Ruthenium hexamine is added to an aliquot of the polymer solution and the combination is stirred for about an hour and then vortexed and centrifuged and then a final mix may be done to generate an intermediate mixture. Then LOx is added to this mixture and stirred for 20 minutes minimally. The resulting viscous solution is then drop-casted at 2 uL or less droplets into the respective wells 1331 and cured at a maximum of 55 C for at least 10 minutes.

According to some embodiments, the choice of various reagents in a sensing film 1300E may allow for the following advantages:
Detection time: 30 sec
>6 month shelf life
<=5 uL sample volume
Reproducibility Sensor to sensor: <5% Lot to Lot: <5%
0.1 mM sensitivity
LOD: 0.2 mM (millimolar)
3.5 mM linearity

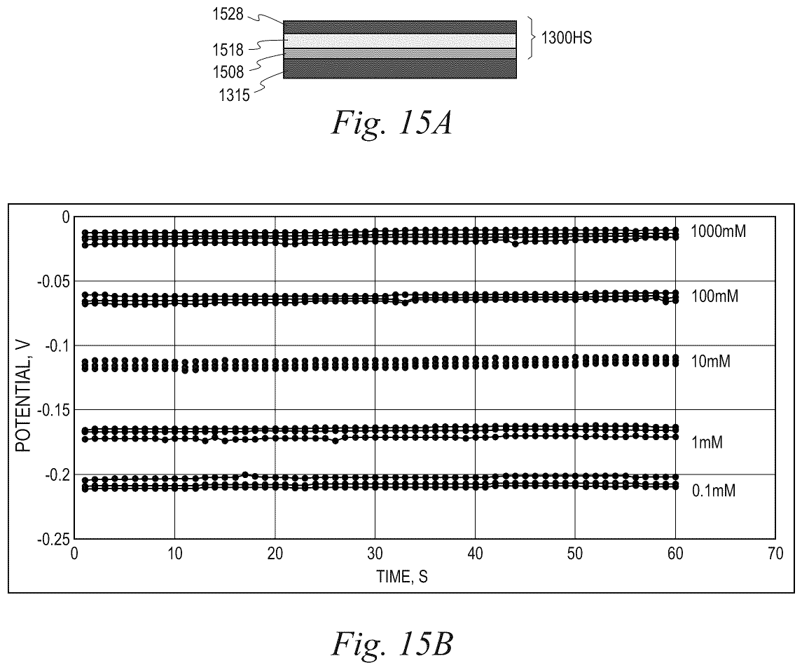

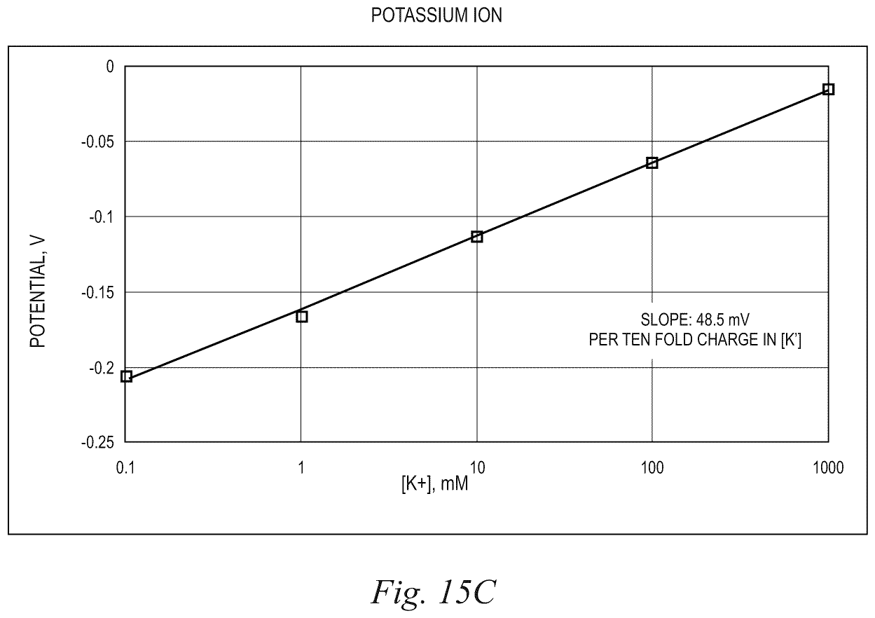

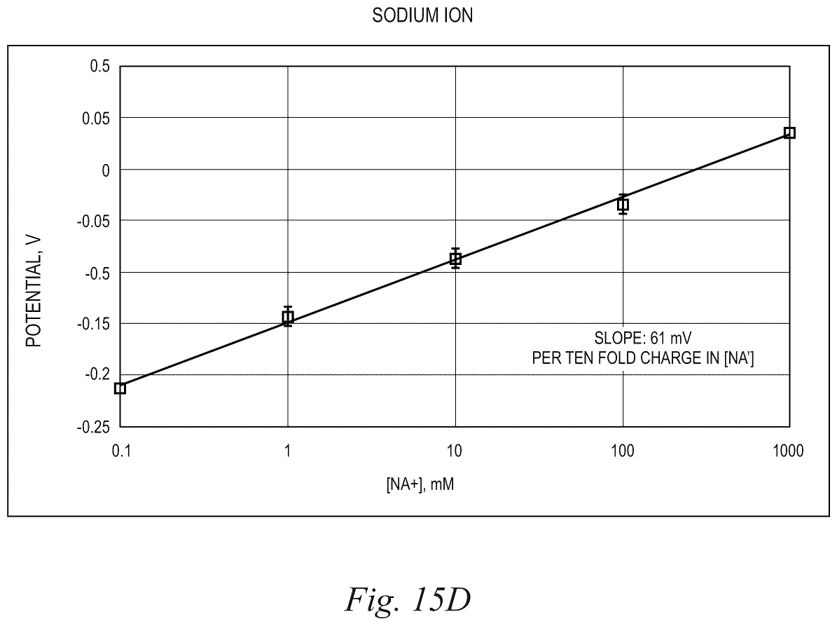

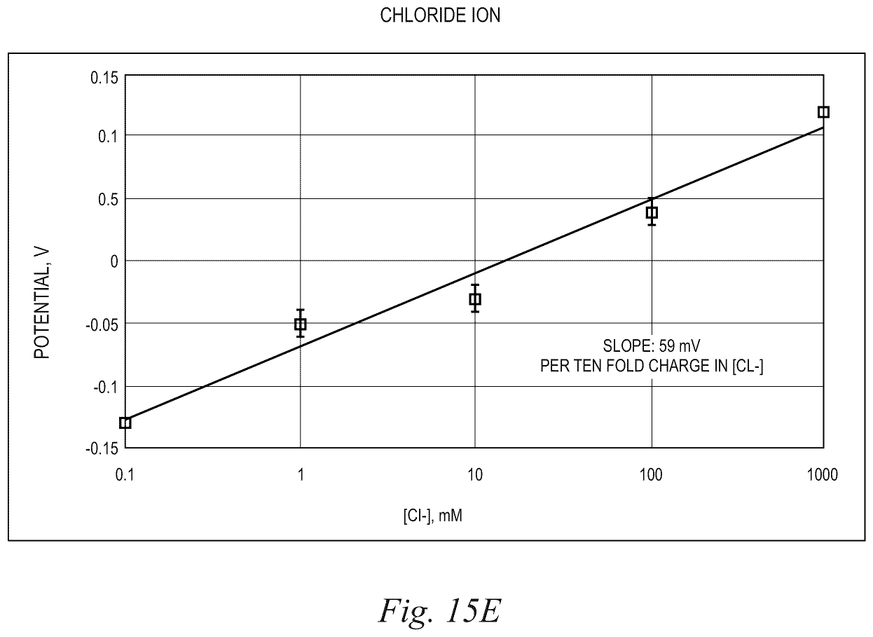

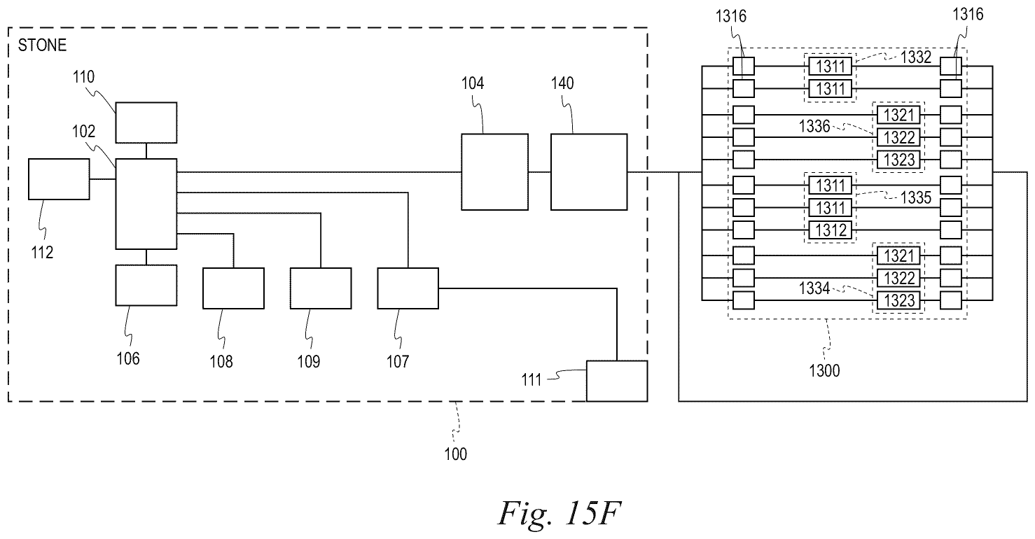

FIG. 15A is a layer stack up of the ion selective hydration sensing film 1300HS for a hydration sensor, or an ion selective electrode generally (ISE) according to some embodiments. The film 1300HS may be drop-cast in layers into the wells 1331, wherein the film 1300HS coats the carbon pads 1311, 1315 (see FIGS. 13D-13F). According to some embodiments, a first layer is a hydrogel layer 1508 which adheres the ion selective hydration sensing film 1300HS to the carbon pad 1311, 1315 and contributes to overall stability. Next is a sensing layer 1518 which contains an ionophore and complimentary salt for the ion of interest, which helps generate a membrane potential used for analyzing ion concentration. A top layer is a PDMS capping layer 1528, which protects the sensing layer 1518 from outside interference and damage and adds to overall stability while allowing for diffusion of ions from solution into the sensing layer for use during analysis.

According to another embodiment, a biosensor chip 1300 may be configured to determine the presence and/or quantity of an analyte using an ion selective reaction, wherein a working electrode detects the activity of an ion species passing through a selective membrane. According to some such embodiments, open circuit potentiometry (OCP) may be used to detect ionic analytes. With reference to FIG. 13E, according to some such embodiments, wells 1334 and 1336 may contain a sensing layer 1300HS to measure the concentrations of sodium (Na) (1334) and potassium (K) (1336) ions, respectively. One of the wells 1334 and 1336 acts as a working well for the sodium (Na) and the other well acts as a working well for potassium (K) ions, According to some embodiments, well 1335 contains two carbon pads that serve as common counter electrodes for some such embodiments, and one Ag/AgCl electrode 1312 as a common reference electrode. The other wells are not used in some such embodiments. A working electrode is where a chemical reaction takes place, including oxidation or reduction. The counter or reference electrodes have opposite chemical reactions occurring at their surfaces, such as oxidation if the working electrode is experiencing reduction. This completes the circuit between the two cells and the potentiostat 104. According to some embodiments, the reference electrode measures the potential of the working electrode without passing current through it, and holds a constant potential throughout the experiment.

According to some embodiments, the sensing film 1300HS is an ion selective electrode (ISE) that may be configured to measure key hydration biomarkers, such as Na+, K+, and Cl−, although the techniques herein can be used to create any selective ISE for any ionic analyte. As illustrated in FIG. 15A, the ion selective hydration sensing film 1300HS may comprise a capping layer 1528 which only contains a polyvinyl alcohol (PVA) film, a sensing layer 1518, and a polymer film 1508 atop a screen-printed carbon electrode 1315. The capping layer 1528 is used to protect the sensing layer 1518 and increase stability, as well as facilitate diffusion of the analyte to the sensing layer 1518.

According to some embodiments, the capping layer 1528 substance is Dowsil 3140, a form of PDMS. According to some embodiments, other types of PDMS could be used if they can be dissolved and drop-cast onto the sensor surface 1518. Other polymers could be used such as Nafion, polyurethane, etc. According to some embodiments, the capping polymer is dissolved in a solvent such as THF, although other solvents can be used such as 2-Methyltetrahydrofuran, Cyclopentyl methyl ether, etc. According to some embodiments, a plasticizer such as DOS is also used in this mixture to drop-cast the polymer. Any other plasticizer such as DEHP, adipates, citrates, phthalates, etc. can be used instead.

According to some embodiments, the sensing layer 1518 is composed of a polymer, ionophore, lipophilic salt and plasticizer. The ionophore acts to capture the analyte ion and allow it to pass through/get trapped in the sensing layer 1518, allowing a potential to be generated that is proportional to the analyte's concentration. According to some embodiments, the ionophore for the Na+ analyte is sodium ionophore X (4-tert-Butylcalix [4] arene-tetraacetic acid tetraethyl ester), but other ionophores for sodium could be any of the Selectophore line of Sigma Aldrich (i.e., Sodium ionophore I, II, etc.). In general, other ionophores for other analytes would be similarly derived molecules with a high affinity for the ion and that cannot diffuse out of the layer 1518. Examples of these would be ETH 1001 for calcium, nonactin for ammonium, Valinomycin for potassium, etc. According to some embodiments, the polymer used for the sensing layer 1518 is PVC. Other polymers such as PVA could be used, as well as conductive polymers such as PEDOT:PSS, PPy, etc. The solvent for the layer 1518 is THF, and the previously mentioned solvents can also be used. According to some embodiments, the plasticizer is also DOS, and previously mentioned alternatives apply. According to some embodiments, the lipophilic salt in this layer 1518 is Na TTFPB (Sodium tetrakis[3,5-bis(trifluoromethyl)phenyl]borate Hydrate). The salt has increased solubility in the layer 1518, which helps solvent extraction of the cation and increases sensitivity of the sensor. Other lipophilic salts for sodium can also include sodium tetrakis(4-fluorophenyl)borate dihydrate, etc. In general, a lipophilic salt that contains the analyte ion would be used for a general purpose ISE. For example, for potassium the salt Potassium tetrakis(4-chlorophenyl)borate could be used.

According to such an embodiment, the sensing film 1300HS and electrode 1315 forms a solid-state electrode, requiring no liquid inner membrane which allows for ease of fabrication and higher stability during storage. It gives immediate results (<1 second) instead of taking up to 10 minutes settling time like conventional ISEs. It has a broad sensitivity range due to its construction and gives a logarithmic Nernstian response.

According to some embodiments, the following advantages may be gained from using a sensing film 1300HS: Stability: 3 months or more at room temp., expecting stability of more than one year after fabrication, sensitivity: 0.1-100 mM in saliva, no calibration, no preconditioning.

According to some embodiments, a sensing film 1300HS is manufactured in the following manner. 2.5% PVA in DI water is prepared, and methanol is carefully added. This solution is drop-cast into the respective well 1331, and then baked at no more than 100 C for 10 minutes minimally. Then a separate solution of sodium ionophore X, Na-TFPB and THF is stirred for 30 minutes or more. PVC is added to this solution and stirred for at least 1 hour. DOS is added to this final solution and stirred for at least 2 hours. The solution is then drop-cast onto the base layer 1508 and baked at no more than 40 C for at least 5 hours. The final solution is made up of Dowsil 3140 in THF and DOS, which is stirred for at least 2 hours. This is drop-cast on to the electrode 1315 and left for an hour at room temperature.

According to another embodiment, a biosensor chip 1300 contains both the enzymatic wells and the ionic wells. In such an embodiment, the wells 1332 and 1333 are used to determine the presence and/or quantity of an analyte using an enzymatic reaction, with well 1332 serving as a background correction well which contains no sensing layer, and well 1333 as the measurement well which is drop-cast with the sensing layer 1300E. According to some embodiments, background correction may be accomplished by subtracting the signal at the background electrode from the signal (current) obtained at the working electrode. Further, both wells 1334 and 1336 and well 1335 contain the sensing layer 1300HS, wherein wells 1334 and 1336 both serve as the working wells for the measurement of Na and K ions, respectively and well 1335 contains the common counter and reference electrode used in the measurement.

FIG. 15B is a graph illustrating the stability response of the sensor to ion-selective reactions, by measuring different concentrations of NaCl over a 1-minute period according to some embodiments. According to some embodiments, the POCT device 100 measures biomarkers in the ion-selective chemistry class via electrochemistry by showing changes in the electromotive force (EMF, volts) with the different concentrations of target ions being monitored under virtually zero current conditions. The FIG. 15B shows the sensor 1300 response to the different concentrations of potassium ions (0.1 to 1000 mM) in the sample. The stability of the sensor 1300 response was evaluated by measuring the response potential as a function of time (over 60 seconds). The results indicate that the sensor 1300 achieves stable potential immediately and is stable over the period of detection. Further, the sensor's ion-selective electrodes show high reproducibility in sensor response towards the detection of target ions at different concentrations.

FIG. 15C-15E are graphs illustrating a response of sensor 1300 at different concentrations of electrolytes, with FIG. 15C showing Potassium, FIG. 15D showing Sodium and FIG. 15E showing Chloride, according to some embodiments. The sensor response to the different concentrations (0.1 to 1000 mM) of Potassium ions (K+) in FIG. 15C, Sodium ions (Na+) in FIG. 15D and Chloride ions (Cl−) in FIG. 15E are presented. For this specific example, the slopes of the linear parts of the response curves are 48.5 mV, 59 mV and 61 mV per decade for K+, Na+ and ions, respectively. Other examples may show alternate response curves. The slopes of the calibration curves are close to Nernstian response and are within the range of experimental error.

FIG. 15F is functional block diagram of a reader/testing device and a sensor according to some embodiments of the present disclosure. The reader or Stone 100 compresses a processor 102 electrically and/or communicatively coupled to a potentiostat circuitry 104 which in turn is electrically and/or communicatively coupled to electrical contacts 160 in the cartridge receptacle 140. The processor 102 may comprise one or more processors and may have one or forms such as being a microcontroller. According to some embodiments, the potentiostat circuitry 104 comprises one or more potentiostats. The potentiostat measures and controls the potential difference between two any two electrodes. In some embodiments in which enzymatic or immunoassay chemical tests are employed, the potentiostat drives a single voltage onto two electrodes and measures the resulting current over time. In some embodiments in which ion selective tests are employed, the potentiostat varies a potential to hold a constant current between a working and reference electrode. The potentiostat then measures the resulting change in potential. The electrical contacts 160 in the cartridge receptacle 140 are electrically coupled to sensor pads or contacts 1316 on the sensor 1300. In turn, these pads or contacts 1316 are electrically coupled to electrical contacts or pads 1311, 1312, 1321, 1322, 1323 which are associated with wells 1332, 1333, 1334, 1335, and 1336 of the sensor 1300. The processor 102 may also be communicatively coupled to a memory 106 which may have operating instructions stored therein and may store information or data received from the processor 102 and/or the chip 1300 and/or an external device, e.g., smartphone 191. According to some embodiments, the electrodes 1311, 1312, 1321, 1322, 1323 on the chip 1300 form part of an electrical circuit monitored by processor 102 and/or potentiostats 104. When a chemical reaction occurs in one or more wells 1331 on the chip 1300, changes in the number of electrons on an electrode 1311, 1312, 1321, 1322, 1323 occur and can be sensed and characteristics of the biosample, e.g., saliva, made be determined by the processor 102. The changes in the number of electrons may be sensed as a change in voltage and/or current in an electrical signal received from an electrode 1311, 1312, 1321, 1322, 1323 by reader 100 such changes being detected by potentiostat(s) 104 and/or processor 102. According to some embodiments, the changes in one or more signals received from one or more electrodes 1311, 1312, 1321, 1322, 1323 are proportional to the amount of one or more substances present in a biosample and the proportional signals are employed by the potentiostats 104 and/or processor 102 to determine and/or calculate a quantity of such substance(s) such as described above. According to some embodiments, changes in one or more signals received from one or more electrodes 1311, 1312, 1321, 1322, 1323 occur when one or more particular substance(s) is(are) present in a biosample and the signals are employed by the potentiostats 104 and/or processor 102 to determine presence of such substance(s) such as described above. The reader 100 also may comprise a memory 106 communicatively coupled to the processor 102. The reader 100 also may comprise a display 110 communicatively coupled to the processor, wherein the processor can cause data or testing results to be displayed on the display. The reader 100 may also comprise an antenna or communication module 108 to permit the reader to be communicatively coupled to one or more external devices such as smartphones, smart watches, or computers. In some embodiments, the communication module comprises Bluetooth hardware, such as, e.g., Bluetooth Low Energy (BLE). The reader 100 may also comprise a power regulation controller 109. The power regulation controller allows for use of a battery 107; and the battery can be charged through a port 111 in a main chassis of the reader. The reader 100 may also comprise a signal conditioning circuit 112 to amplify a detected electrical signal from the biosensor chip.

C. Manufacturing of Enzymatic and/or Ion Selective Sensors

FIG. 16A is a top or plan view of a printed layer strip 1610 which comprises a plurality of screen-printed electrode layers 1310 which can be used to make a plurality of sensor chips 1300 according to some embodiments of the present disclosure. The manufactured strip 1610 comprises a plurality of screen-printed electrode layers 1310 shown in FIG. 13C. According to some embodiments, a plurality of screen-printed electrode layers 1310 are printed in strips 1610 with alignment holes 1612 for precise mounting for addition of the spacer 1340 and top cover layers 1350. Printing in strips allows for high scaling of production and ease of automation in manufacturing of the electrodes 1311, 1312 and chips 1300, as they can be cut in standard sizes from very large, laser cut strips 1610 of material. During manufacture, the one or more alignment holes 1612 of a printed layer strip 1610 are placed about one or more alignment posts.

FIG. 16B is a top or plan view of a strip 1640 of a spacer layers 1340, used for automated manufacturing according to some embodiments of the present disclosure. The manufactured spacer strip 1640 comprises a plurality of spacer layers 1340 shown in FIG. 13B. According to some embodiments, the spacer strip 1640 has one or more alignment holes 1642 for precise alignment with additional strips such as a printed layer strip 1610 and a top cover layer strip 1650. During manufacture, the one or more alignment holes 1642 of a spacer strip 1640 are placed about the one or more alignments posts and the spacer strip 1640 is covered with a thin adhesive layer and pressed onto the printed layer strip 1612. Exemplary dimensions shown in FIG. 16B are in millimeters (mm). According to some embodiments, the spacer strip 1640 is made of PET, and laser cut to allow for a trough to form over each well 1331 for increased saliva retention and to strengthen the electrode material by making it more rigid. The spacer 1340 also makes space between the screen-printed electrode layers 1310 and a top cover 1350 shown in FIG. 13B for saliva to flow correctly.

FIG. 16C is a top or plan view of a top cover strip 1650 comprising a plurality of top covers 1350 shown in FIG. 13B, used for automated manufacturing according to some embodiments of the present disclosure. According to some embodiments, the top cover strip 1650 is made of a hydrophilic plastic film. The hydrophilicity of the film redirects the saliva along the film into each of the wells 1331, as well as protects the electrodes 1311, 1312 within each well from outside interference. According to some embodiments, the top cover strip 1650 has one or more alignment holes 1652 for precise alignment with additional strips such as a printed layer strip 1610 and a spacer strip 1640. According to some embodiments, during manufacture, the one or more alignment holes 1652 of a cover strip 1650 are placed about the one or more alignments posts and the top cover 1350 is attached to a spacer strip 1640 using a pressure activated adhesive.

According to some embodiments, the biosensor chips 1300 are manufactured in a way such that the chips 1300 are cheap and reproducible. In such embodiments, a printed layer 1310 is first manufactured using screen-printing in a strip design 1610. Then this design is mounted such that the holes 1612 of the strip 1610 can line up across all three layers that will be pressed on, which include 1640 and 1650 aligning the holes 1612, 1642, and 1652 of the respective layers. A spacer strip 1640 is pressed onto the printed layer strip 1310, such that a pressure activated adhesive bonds the layers 1610, 1640 together. Similarly, a top cover strip 1650 is pressed onto the spacer layer 1640, and then the combined layers 1610 are cut or divided into individual biosensor chips 1300.

D. Immunoassay Sensors

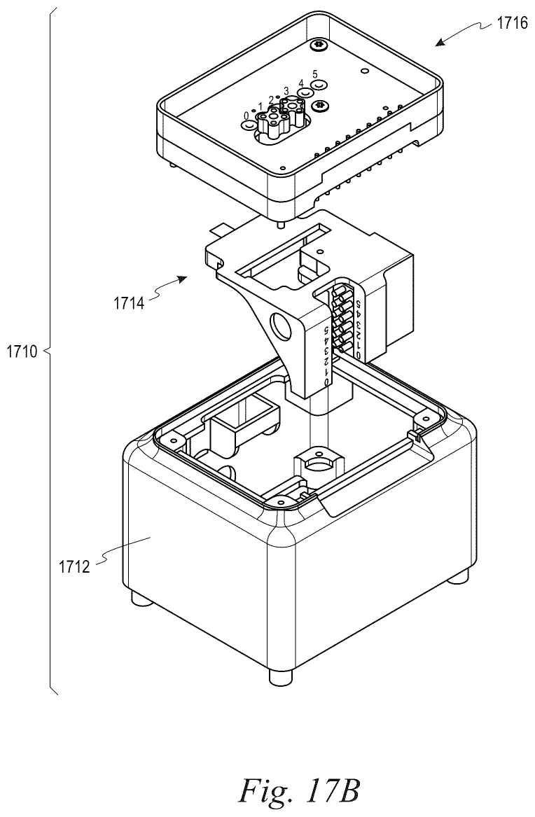

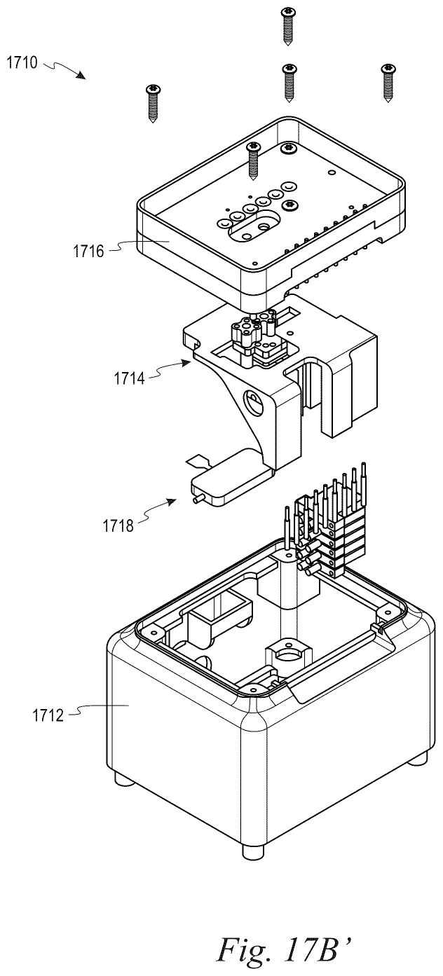

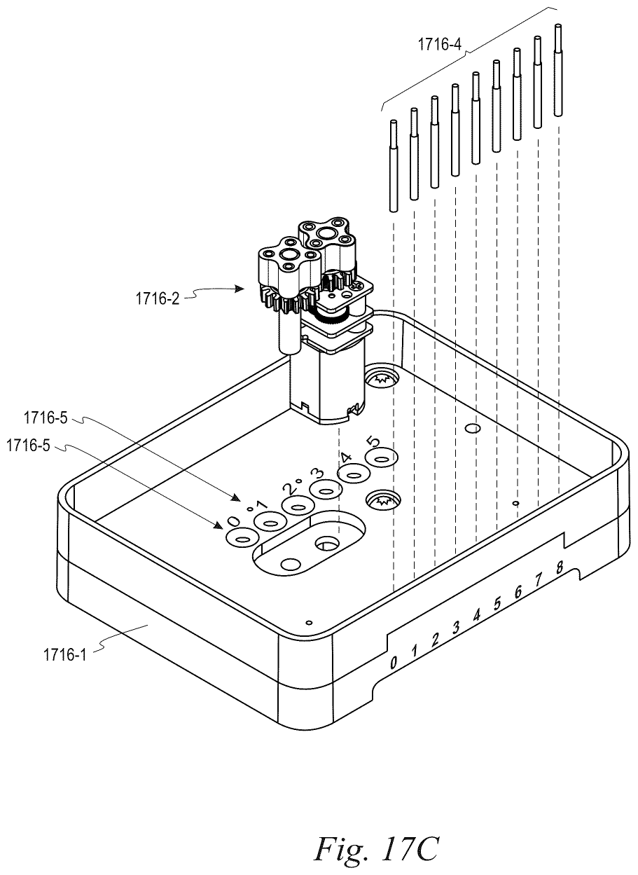

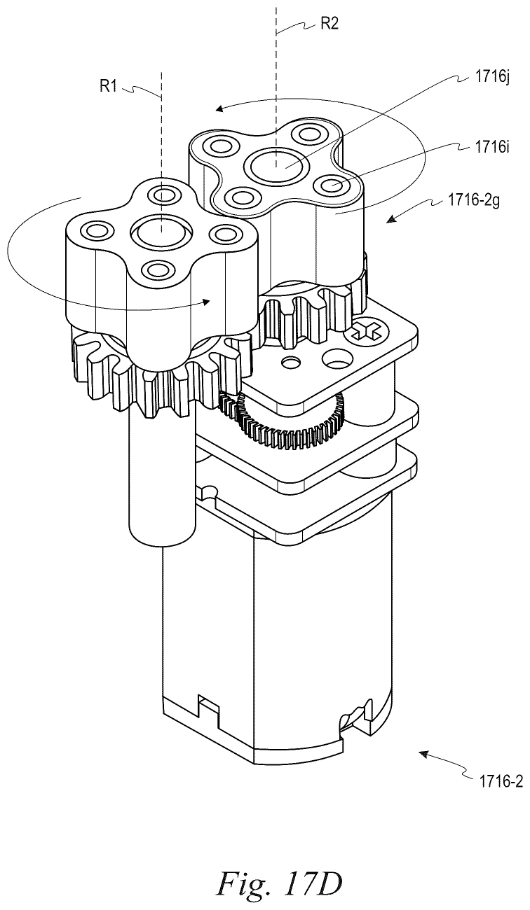

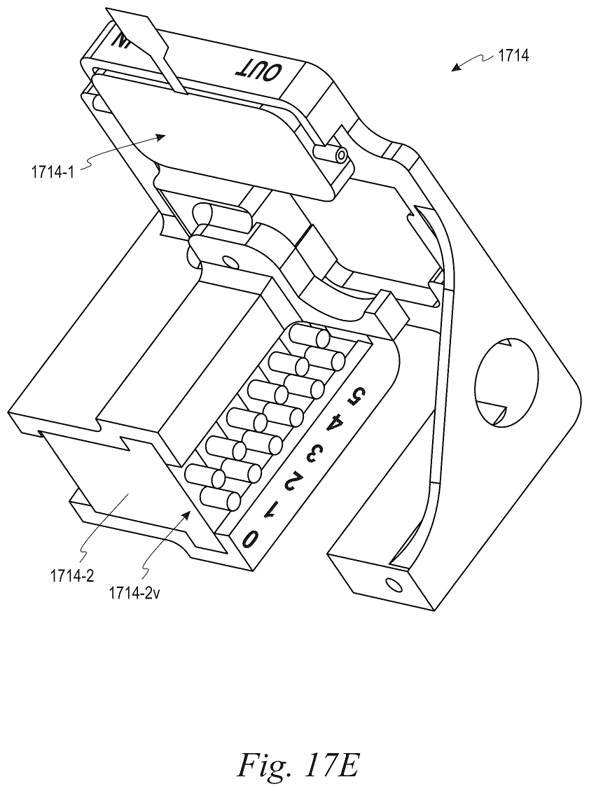

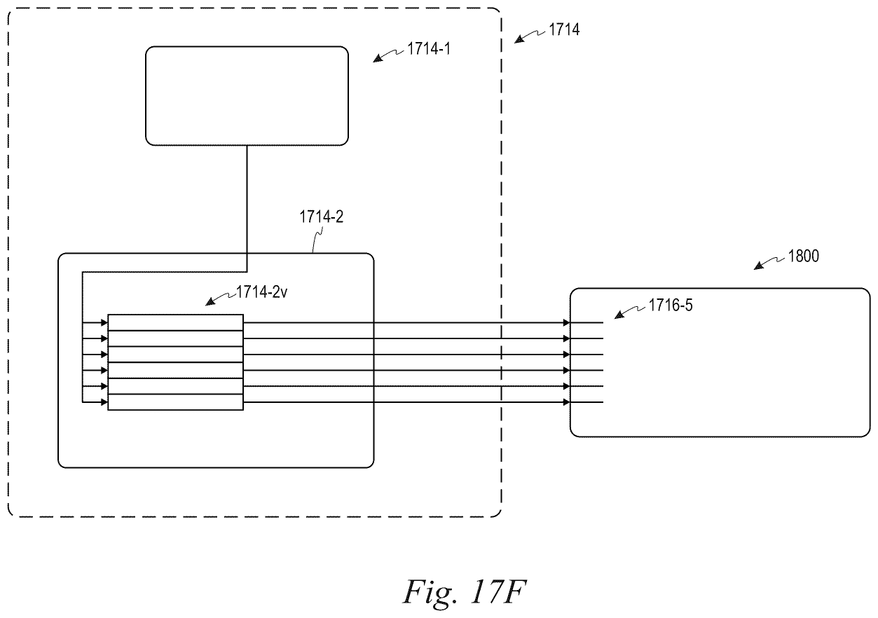

FIG. 17A is an exploded perspective view of an immunoassay system or platform 1700 according to some embodiments of the present disclosure. According to some embodiments, the immunoassay system 1700 comprises a Cube reader 1710, a disposable microfluidics chip 1800, and a biofluid, e.g., saliva, collector 400.

FIG. 17B is an exploded perspective view of a component of the Cube reader 1710 according to some embodiments of the present disclosure. FIG. 17B′ is an exploded perspective view of a component of a Cube reader 1710 according to some embodiments of the present disclosure. One component of an immunoassay system 1700 is a Cube reader 1710, which houses electronics and actuators used to complete an analysis of a biofluid such a saliva according to some embodiments. According to some embodiments, the system 1700 can include disposable devices such as a microfluidic chip 1800 and the saliva collector 400.

According to some embodiments, the immunoassay system 1700 and the Cube reader 1710 comprise a base 1712, a manifold 1714 and a test bed 1716.

According to some embodiments, the base 1712 comprises tubes and wiring.

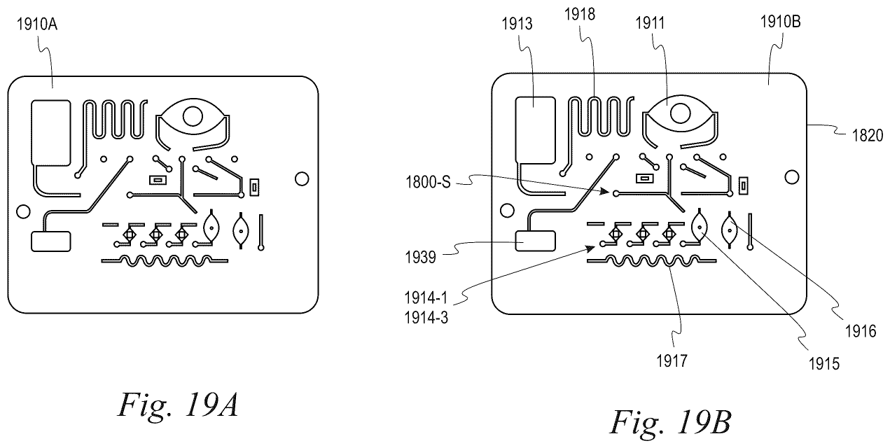

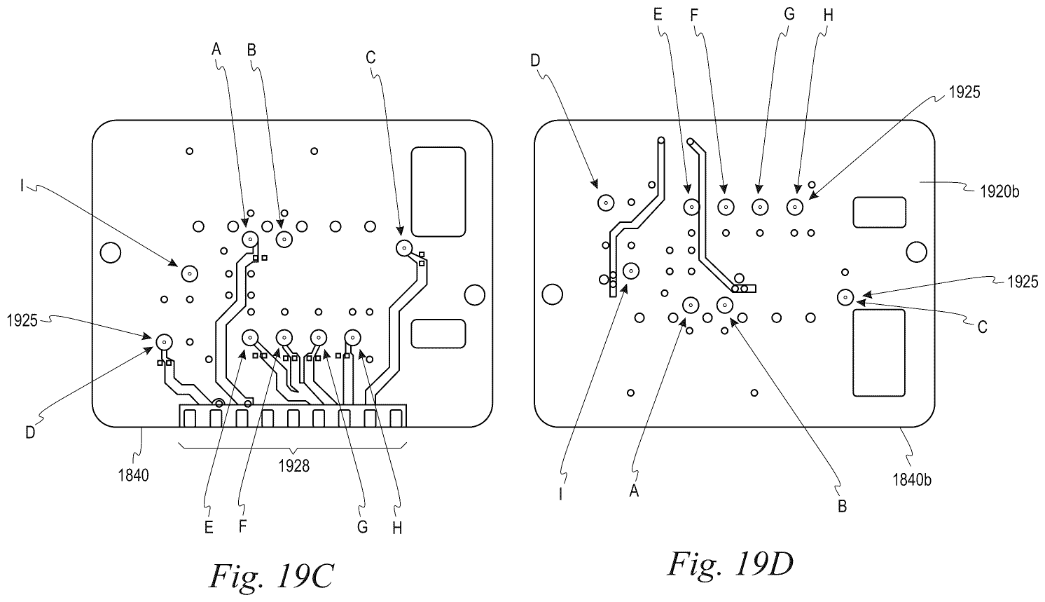

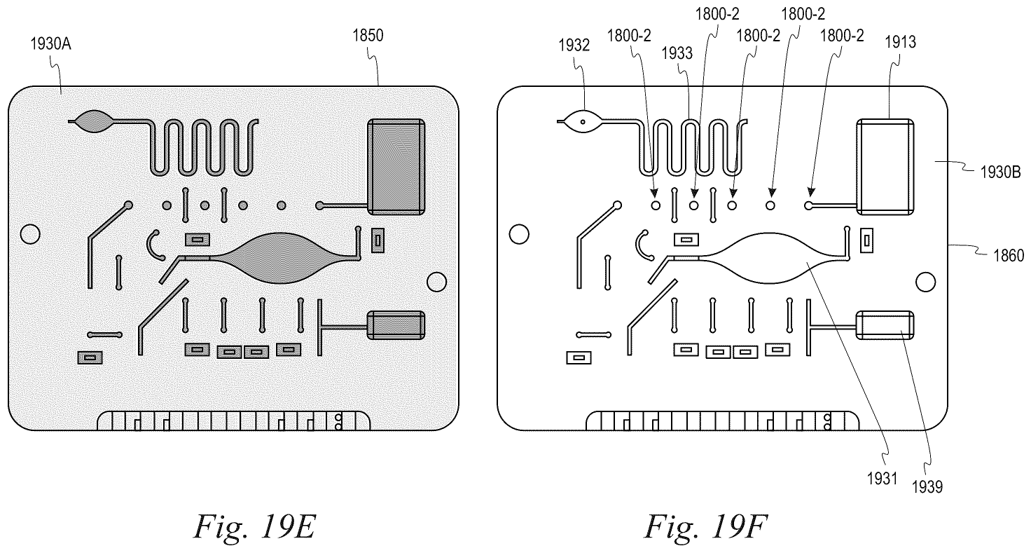

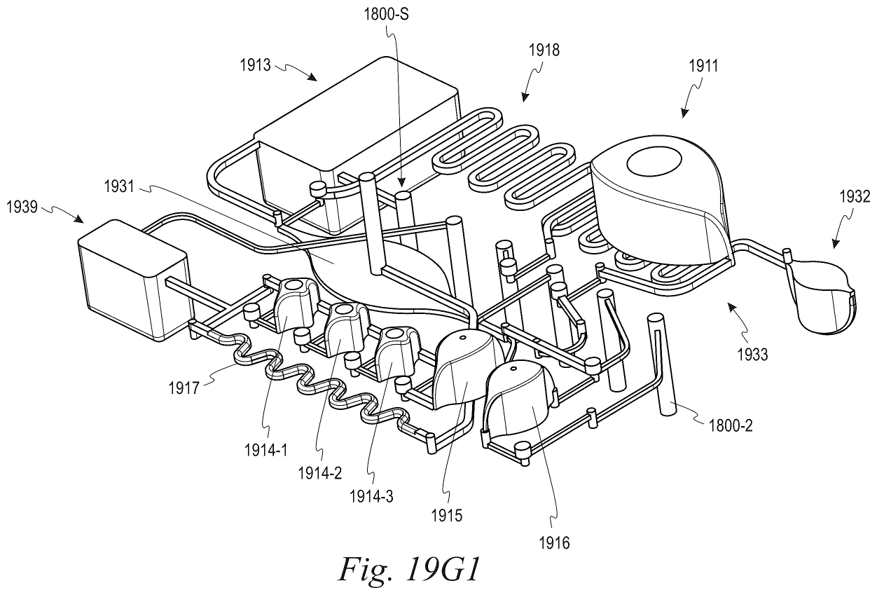

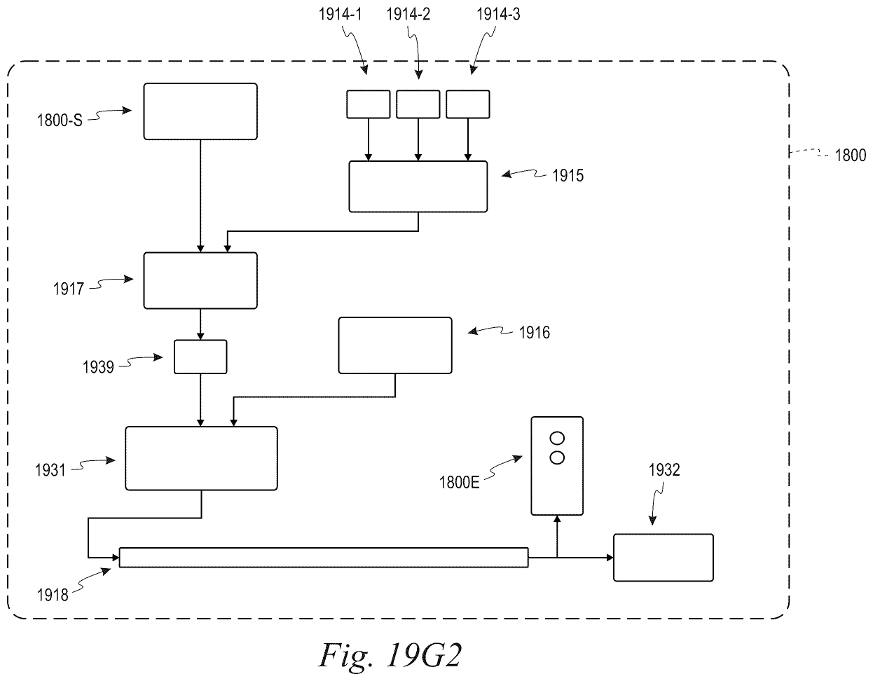

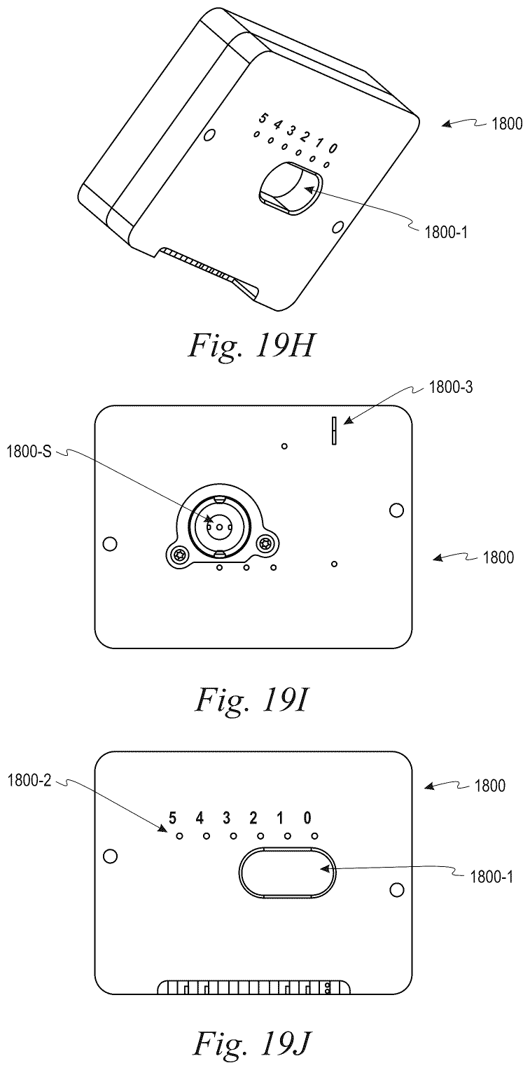

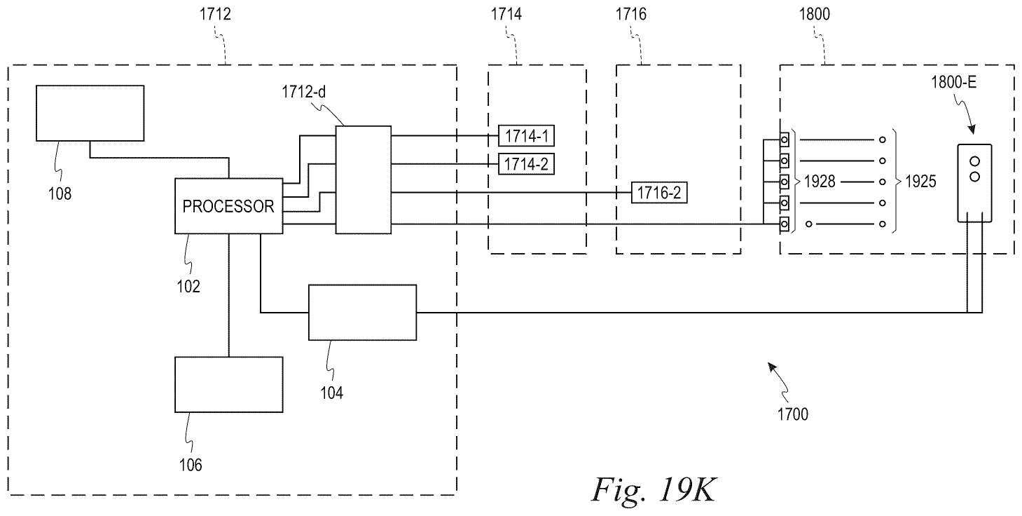

FIG. 17C is a perspective view of a test bed 1716 according to some embodiments. According to some embodiments, the test bed 1716 comprises a test bed housing 1716-1, a magnetic gear assembly 1716-2, and Pogo pins 1716-4. The test bed housing 1716-1 has a plurality of air ports 1716-5 therein that correspond to air inlets 1800-2 (see FIG. 19J) and valves 1714-2.

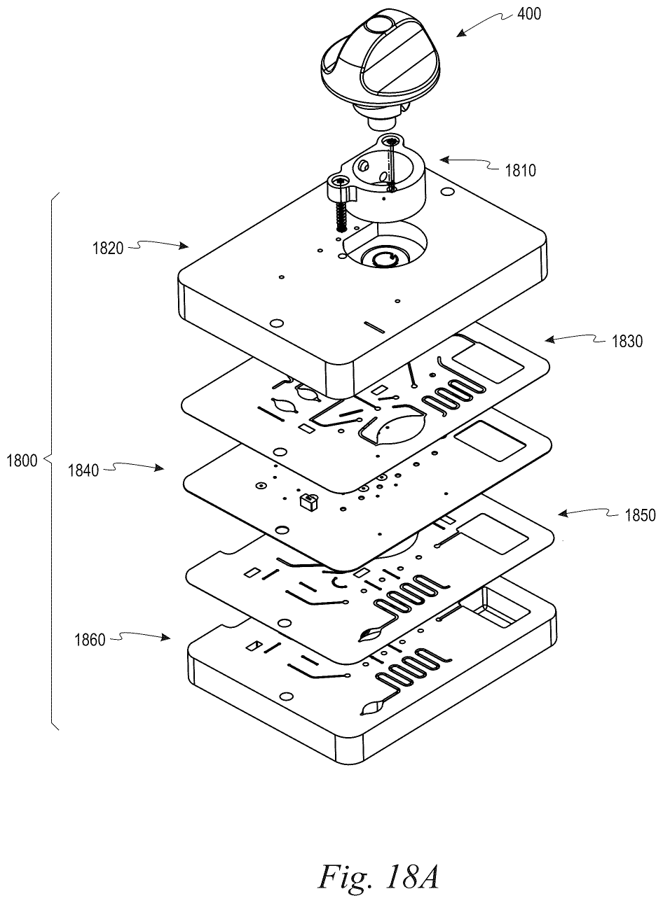

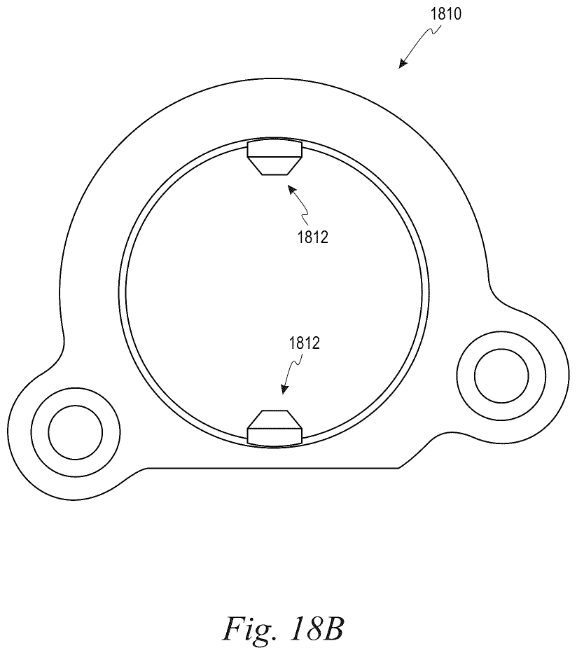

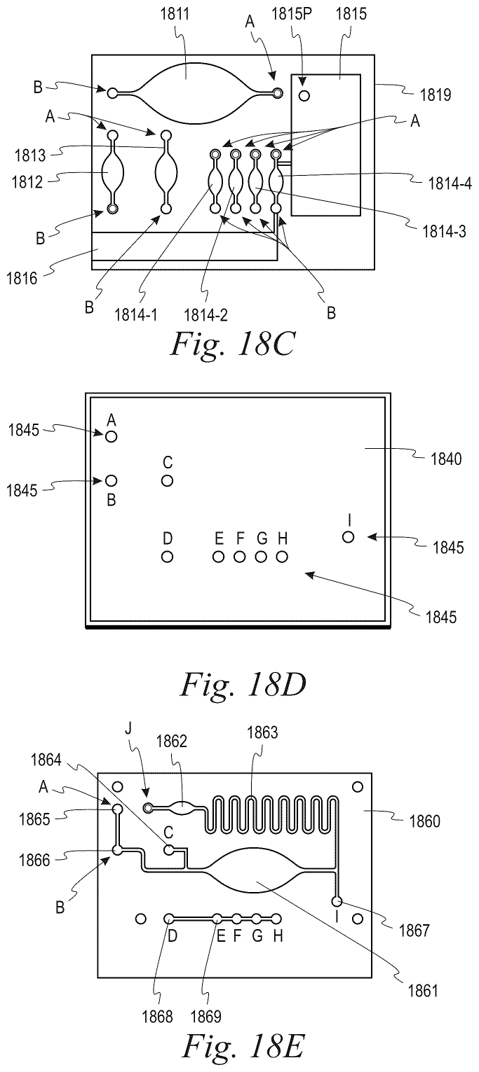

FIG. 17D is a perspective view of a magnetic gear assembly 1716-2 having magnetic gears 1716-2g according to some embodiments. Magnetic gears 1716-2g insert into a bottom chip port 1800-1 (see FIGS. 1911, 19J) and rotate to mix the substrates in a reaction chamber 1931 (see FIGS. 18C, 18E, 19F, 19G1) through the action of the ferromagnetic beads. Neodymium magnets 1716-j are located in the magnetic gears 1716-2g, which by rotating affect a changing magnetic field on the reaction chamber 1931 to mix the reactants by causing the ferromagnetic beads within the reaction chamber 1931 to move. In some embodiments, magnets can also be placed into the bed 1716-1 to attach the chip 1800 to the test bed 1730 tightly so there is a seal along the air ports 1716-5. The pogo pins 1716-4 correspond to the contacts 1928 (see FIG. 19C) on the chip 1800. When the chip 1800 is clamped to the test bed surface 1716-1s due to the force from the magnets 1716-3, the pogo pins 1716-4 are compressed. These pins 1716-4 pass along the voltage from a processor or microcontroller 102 to melt the wax valves 1925.

FIG. 17E is a perspective view of a manifold 1714 according to some embodiments. According to some embodiments, the manifold 1714 comprises a micropump 1714-1 that moves fluid along the entire device 1700. According to some embodiments, the micropump 1714-1 uses low voltage and is piezoelectric. For example, the micropump 1714-1 may be a Bartels mp6 micropump. According to some embodiments, the manifold 1714 comprises a valve manifold 1714-2 comprises individual microvalves 1714-2v. These microvalves 1714-2 may be shape-memory alloy valves (e.g., valves made by Takasago Electric, Inc. SMV-2R-AN1F) that open in response to voltage signals from the microcontroller 102. They allow input—output for the fluids and air throughout the device 1700. These microvalves 1714-2v may be stacked into the manifold 1714 and correspond to the numbers of each port in the test bed 1716-1, e.g., the 0 valve may correspond to the line supplying 1716-5 that is labelled 0 as well.

FIG. 17F is a schematic depicting fluidic flow in a manifold 1714 and a microfluidics chip 1800 according to some embodiments. As shown in FIG. 17F a pump 1714-1 is connected via a single line to a valve manifold 1714-2, through which the air pumped from the pump is carried through an individual line to the valves 1714-2v and to the corresponding air ports 1716-5 in the chip 1800. If a valve 1714-2v in the valve manifold 1714 is labelled “0” for instance, that number corresponds to the entire line in the device that ends at the corresponding air port 1716-5 labelled “0”, and is the same for all lines from 0-5, in some embodiments. The pump 1714-1 and/or valves 1714-2v may be communicatively coupled to a processor 102 which may the operation of the same. This control from pump 1714-1 to chip 1800 allows for different routes in the microfluidic chip to be selectively utilized for steps in the experiment.

Example systems can employ one or more or all of the following features: small sample volumes (e.g., ≤ about 20 uL), 30 second measurement time, under 12-minute processing time instead of hours, flow metering in situ, active pumping in microfluidics instead of capillary flow, self-contained disposable cartridges, heating for melting wax seals on reagent container.

According to some embodiments, the system is provided having one or more or all of the below features:
Small scale, multilayer microfluidic designs for small scale immunoassay;
Immunoassay POC using saliva vs. in lab;
Unique processing steps (reagents, mixing etc.);
Low sample volume (20 uL vs 200 uL (nearest competitor));
30 second measurement time;
Less than 12-minute processing time;
Locking mechanism for the handle integration into cartridge;
Self-contained, disposable cartridges that do all the analysis; and/or
Wax melted using heating electrode on the substrate.

Microfluidic Biosensor Chip 1800:

FIG. 18A is an exploded perspective view of an immunoassay biosensor chip 1800 according to some embodiments. As illustrated, the biosensor chip 1800 comprises a biofluid, e.g., saliva, collector adapter 1810 for receiving a biofluid collection device 400, a first or top microfluidic layer 1820 (see also FIGS. 18D, 19A-19B), a first or top adhesive layer 1830 (see also FIGS. 18E, 19A-19B), a wax valve PCB 1840 (see also FIG. 19C), a second or bottom adhesive layer 1850 (see also FIGS. 19E-19F), and a second or bottom microfluidic layer 1860 (see also FIGS. 19E-19F).

FIG. 18B is a plan view of a biofluid, e.g., saliva, collector adapter 1810 for receiving a biofluid collection device 400. The biofluid collector adapter 1810 comprises one or more thread pins 1812. The thread pins 1812 serve the same purpose as protrusions or thread pins 712 described above (see FIG. 7D), allowing the handle 450 to thread and put pressure onto the cotton swab tip 480 to release a biofluid such as saliva into the chip 1800.

FIGS. 18C-18E are top view of a multiple layers of an immunoassay biosensor chip 1800 according to some other embodiments of chip 1800. According to some embodiments, there are three layers to the chip: a microfluidic channel layer 1860, a wax seal layer 1840, and a sample storage layer 1819.

FIGS. 18C-18E top views of multiple layers of an example chip 1800 according to a first embodiment of chip 1800.

FIGS. 19A-19B and 19E-19F are top views of multiple layers of an example chips 1800 according to a second embodiment. FIGS. 19C-19D shows example heating PCBs 1840, 1840b illustrating example wax valves 1925 thereon (individually labeled A-I). In some versions of the second embodiment, only heating PCB 1840 shown in FIG. 19C is employed.

The first embodiment of chip 1800 will be describe with reference to FIGS. 18C-18E.

Sample storage layer 1819 (FIG. 18C): The storage layer 1819 contains reagents, as well as stores waste after analysis and mixing of a sample with an antibody. According to some embodiments, the sample storage layer 1819 comprises a wash buffer reservoir 1811, a waste collection chamber 1815, a TMB reservoir 1812, a sample mixing chamber 1811, a plurality of sample storage chambers 1814-1, 1814-2, 1814-3, a saliva collector interface 1816, and a saliva metering port 1814-4.

Wax Seal Layer 1840 (FIG. 18D): The wax seal layer 1840 holds valves 1825 (labeled A-I) that are positioned between the storage layer 1819 and the microfluidic layer 1860. The wax valves 1845 are melted during analysis to release the reagents stored above.

Microfluidic Layer 1830 (FIG. 18E): The microfluidic layer 1860 contains a “lab” for analysis. According to some embodiments, this layer 1860 comprises a reaction chamber 1861, a serpentine mixing channel 1863 for mixing a biofluid sample with TMB, a detection chamber 1862, a sample inlet port 1864, an outlet to a waste reservoir 1867, an air/wash buffer inlet 1865, and a TMB inlet 1866.

Referring to FIGS. 18A-18E and FIGS. 19A-19J, according to some embodiments, each chip 1800 contains a microfluidic “lab” complete with microfluidic mixing, reacting and storage channels and its own reagents. The reagents are stored in the chip 1800 and released into the cartridge microfluidic system during analysis using paraffin wax valves 1845/1925 that are melted using a heating PCB 1840, 1840b in the chip 1800.

The second embodiment of chip 1800 will be describe with reference to FIGS. 19A-19J.

FIG. 19A shows an adhesive layer that is cut to match the perimeters of the microfluidic routes shown in FIG. 19B to seal the chip together. The adhesive layer of FIG. 19A is positioned between the layers shown in FIGS. 19B and 19D.

FIG. 19B shows a first or top half of an immunoassay chip 1800. This layer of the chip comprises a main waste chamber 1913, a serpentine channel 1918 for mixing and TMB reaction, as well as controlling signal read delay by slowing down fluid flow. The layer also comprises a wash buffer storage chamber 1911, a TMB storage chamber 1916, a pre-mixing chamber 1915 for an antibody, ferromagnetic beads and HRP. The layer also comprises a long serpentine mixing channel 1917 to thoroughly mix a solution before reactants go into a reaction chamber, an antibody storage chamber 1914-1, a magnetic bead storage chamber 1914-2, and a HRP storage chamber 1914-3. The layer also comprises an inlet 1800-S for receiving saliva or other biofluid from the biofluid collector adapter 1810.

FIG. 19C shows a first side of a Wax valve PCB layer that responds to electrical inputs to melt wax, releasing sealed fluids for use in a reaction. Electrical contacts 1928 connect to wax valves 1925. When an electrical signal is transmitted along a respective connection, the wax seal in a corresponding valve 1925 melts, thereby releasing a corresponding stored fluid.

FIG. 19D shows a second, opposing side of the Wax valve PCB layer of FIG. 19C.

FIG. 19E shows an adhesive layer that is cut to match the perimeters of the microfluidic routes shown in FIG. 19F to seal the chip together. The adhesive layer of FIG. 19F is applied to the half of an immunoassay chip 1800 shown in FIG. 19E and the combination of the adhesive layer of FIG. 19E and the half of an immunoassay chip 1800 shown in FIG. 19F is then pressed against the first half of the Wax valve PCB layer shown in FIG. 19C.

FIG. 19F shows a second or bottom half of an immunoassay chip 1800. This half of the immunoassay chip 1800 comprises a chamber 1932, a waste chamber 1913 to collect reacted TMB, a mixing channel 1933 for mixing TMB and slowing flow of a solution to chamber 1932, a sample metering chamber 1939 and a main reaction chamber 1931. Air ports 1800-2 allow the flow of air from the pump into the chip fluidic network. These correspond with their respective air lines (0-5).

Referring to FIGS. 19G1 and 19G2, saliva is input at 1800-S. Capillary forces move the saliva into a mixing channel 1917, along with the antibodies, magnetic beads and HRP from the chambers 1914-1,1914-2,1914-3 respectively. These first combine in chamber 1915 before being pumped to the mixing channel 1917. The combined sample fluid is pumped into the sample metering chamber 1939, then pumped to the main reaction chamber 1931. The magnetic motor 1716-2 spins underneath or adjacent chamber 1931, mixing the combined fluids thoroughly and adhering the antibody—bead—HRP—cortisol complex to the bottom of the reaction chamber 1931. Buffer is then pumped in from buffer storage or wash chamber 1911 to remove unconjugated material, which is pumped to waste chamber 1913. TMB is then pumped from TMB storage chamber 1916 into main reaction chamber 1931 to react with the conjugated material. The solution mixed with TMB is then passed into serpentine mixing channel 1918 for further mixing and to slow down the flow speed, where it is pumped to an electrode 1800-E for measurement. Any excess fluid is pumped into waste chamber 1913.

According to some embodiments, the chip 1800 works as follows: in response to a signal from microprocessor 102, a 5V signal is sent through traces into heating resistors of 25Ω, with a power rating of 1.5 W. The resulting power dispersed at each resistor is distributed as heat to copper through holes which contain a thin layer of paraffin wax. The resulting heat melts a corresponding wax valve 1845, 1925, allowing for the reagents or fluids contained in the storage layer of the microfluidic chip 1800 to be dispensed into the microfluidic reaction layer 1860. Each valve 1845, 1925 is controlled by a separate trace to a microcontroller 102 of the immunoassay Cube reader 1710, allowing for precise dispensation of each chemical when needed in the reaction sequence.

Turning to FIG. 19G1, is a perspective view illustrating microfluidic paths and wells as within a disposable microfluidics chip 1800 according to some embodiments. These correspond to the paths shown in FIGS. 19A-19B, but as they would be when the chip is sealed, forming these paths and chambers.

FIG. 19G2 is a block diagram representing the physical flow of fluids through FIG. 19G1A.

FIG. 19H is a perspective bottom view, FIG. 19I is a top view, and FIG. 19J is a bottom view of an assembled microfluidics chip 1800 according to some embodiments. A bottom chip port 1800-1 allows for the magnetic gears 1716-2 to insert and get very close to the reaction chamber 1831, 1931, which sits on the opposite side of this surface. Chip air inlets 1800-2 allow for the micropump 1714-1 to move fluid around the microfluidic chambers. An electrode port 1800-3 may be a standard carbon electrode 1800-E is inserted for analysis of a reaction in the chip 1800.

According to some embodiments, the top microfluidic layer 1820 of the microfluidics chip 1800 may comprise a main waste chamber 1913. Reactant waste is routed to the main waste chamber 1913 and contained for disposal. The top microfluidic layer 1820 may also comprise a wash chamber 1911—a wash step is performed here, where reagent is passed over the magnetically adhered antibody complex to get rid of un-adhered reactant. The top microfluidic layer 1820 may also comprise a sample metering and overflow chamber 1939 (see FIG. 19B). Extra saliva (in excess of what is required for analysis) is sent to the sample waste chamber 1939. The top microfluidic layer 1820 also may comprise an antibody storage chamber 1914-1, a magnetic bead storage chamber 1914-2, and an HRP storage chamber 1914-3. The antibody storage chamber 1914-1 stores an antibody. When its valve is melted, the antibody goes into the mixing chamber 1915. The magnetic bead storage chamber 1914-2 stores magnetic beads for mixing. The HRP storage chamber 1914-3 stores HRP for mixing. These three chambers are released with separate wax valves into mixing chamber 1915, and then sent to mixing channel 1917 to mix with the sample. The top microfluidic layer 1820 also may comprise a pre-mix chamber 1915 in which premixed reactant/sample is stored and a pre-mix chamber 1916. The top microfluidic layer 1820 may also comprise a sample metering chamber 1939 to assist with monitoring the amount of sample loaded into the reaction chamber 1931. The top microfluidic layer 1820 may also comprise a TMB mixing channel 1918 which may be a serpentine-contoured channel for mixing of the TMB and the HRP-antibody-bead-cortisol conjugate inside the reaction chamber 1931.

According to some embodiments, the top adhesive layer 1830 of the microfluidics chip 1800 may be made of plastic (PET or other thin, flexible plastic) covered on both sides with pressure-sensitive adhesive. The plastic may be laser cut with the pattern shown (see FIGS. 18A, 19A and 19E). This adhesive binds the top layer 1820 to the PCB layer 1940. The adhesive layer 1830 and the top layer 1820 layer are hydrophobic, which keeps the liquids in the microfluidic channels instead of leaking out.

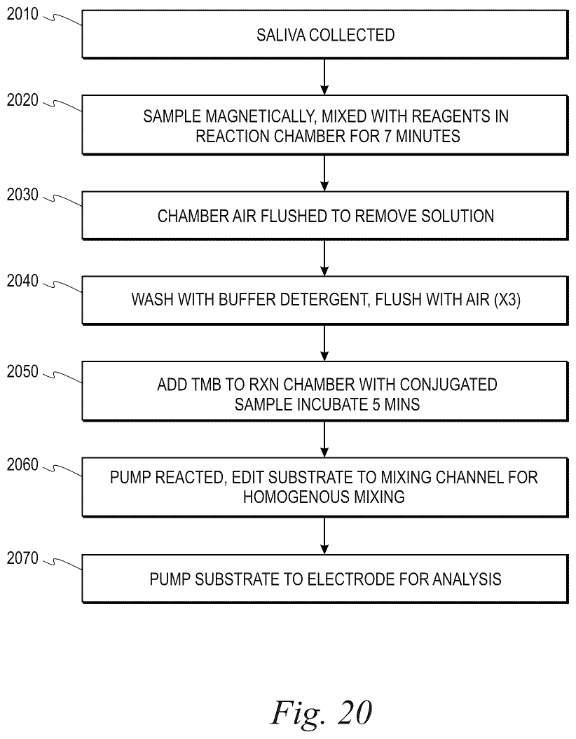

According to some embodiments, the wax valve PCB 1840 comprises wax valves 1925 and contacts 1928. The PCB 1840 may be a thin (0.6 mm) board that contains wax vias 1925. PCB 1840 sits between the top 1820 and bottom 1860 microfluidic layers and forms the surface for the cavities and channels on each layer. The wax vias 1925 may be copper vias of various diameters that are covered in liquid paraffin wax and allowed to dry. When a signal comes in from the processor or controller 102 through the contacts 1928, a voltage is run through a resister connected to a corresponding one of the vias 1925, which generates heat as the resistors have a small resistance value (<10 ohm). The heat is transferred to the corresponding via from a direct contact and within a second the wax in the via melts away, allowing fluid to flow through the via such as a corresponding specific chemical or sample to flow through the top microfluidic layer 1820 to the bottom layer 1860. The opening of the respective wax valves 1925 can be controllable, triggered such as one at a time, such that a sequence of chemicals/fluids is released and used throughout the process (described in the flowchart—see FIG. 20).

According to some embodiments, the bottom adhesive layer 1850 of the microfluidics chip 1800 may be made of plastic (PET or other thin, flexible plastic) covered on both sides with pressure-sensitive adhesive. The plastic may be laser cut with the pattern shown (see FIGS. 19A and 19E). This adhesive binds the bottom layer 1860 to the PCB layer 1940. The adhesive layer 1850 and the bottom layer 1860 layer are hydrophobic, which keeps the liquids in the microfluidic channels instead of leaking out.

According to some embodiments, the bottom microfluidic layer 1860 of the microfluidics chip 1800 may comprise a reaction chamber 1931. According to some embodiments, a TMB reaction occurs in the reaction chamber 1931, and the chamber 1931 allows for pumping the final reacted fluid onto the electrode 1800-E. The chamber 1931 allows insertion of a standard carbon screen printed electrode, and fluid is pumped into the chamber such that the electrode is covered in liquid so that the chamber may act as an electrochemical cell for measurement. The bottom microfluidic layer 1860 may also comprise a TMB mixing channel 1933 which may have a serpentine contour. The bottom microfluidic layer 1860 may also comprise a spill over chamber 1932 which may provide additional storage space for spill over from any liquid in excess of what is needed to measure on 1800-E. 1800-E may be located across mixing channel 1933 before chamber 1932. The bottom microfluidic layer 1860 may also comprise a main waste chamber 1938 and a sample waste chamber 1939.

According to some embodiments, the chip 1800 works as follows: a 5V signal is sent through traces into heating resistors of <25Ω, with a power rating of at least 1.5 W. The resulting power dispersed at each resistor is distributed as heat to copper through holes which contain a thin layer of paraffin wax. The resulting heat melts the wax valve(s) 1925, allowing for the reagents or fluids contained in the storage layer 1820 of the microfluidic chip 1800 to be dispensed into the microfluidic reaction layer (composes all of FIG. 19G1, the reactants/substractes can be released into the system. Each valve 1825 is controlled by a separate trace to a microcontroller 102 of the immunoassay Cube (1700), allowing for precise dispensation of each chemical when needed in a reaction sequence.

FIG. 19K is functional block diagram of electrical components of an immunoassay system 1700 according to some embodiments of the present disclosure. 1712-d is a functional block of driver to run the valves/pump in 1714 and 1716. Processor 102 sends out signals/instructions to 1712-d to drive pumps, valves or melt the wax vias based on parameters set during execution. It also sends data to communication module 108. Potentiostat 104 measures the output of the chemical reaction at electrode 1800-E and sends the data/signals back to processor 102.

CHEMISTRY PROCESS:

The immunoassay platform functions by way of microfluidic mixing and sampling. First, a sample (saliva, although could be other biofluids such as blood or urine in other embodiments) is added to the system through the collector adapter 1810 using the biofluid collection device 400. Stored reagents (Ab, enzyme tagged competitive agent, immobilizing substrate particle) are mixed with the sample. These are moved to a reaction chamber 1831 and reacted for 7 minutes, during which the antibody will conjugate with the streptavidin-tagged magnetic particle. Then, binding competition occurs between the analyte present in the saliva and the competitive agent (analyte tagged with enzyme). The reaction chamber 1831 is then purged with air and washed with the phosphate buffer, leaving only the conjugated magnetic particles. TMB is added and incubated with the particles for at least 5 minutes, and then pumped to a mixing channel 1833 for homogenous mixing. This mixture is then sent to the electrode 1800-E for analysis.

Example reagents include: Biotinylated polyclonal rabbit anti-cortisol antibody in stabilizing buffer, cortisol-3-CMO-HRP in HRP stabilizer, magnetic particle with streptavidin conjugate in phosphate buffer, phosphate buffered saline with 0.05% Tween 20, [3,3,5,5′]-Tetramethylbenzidine.

A general immunoassay platform 1700 includes a combination of reagents to facilitate electrochemical detection. Generally, these are a biotinylated antibody specific to the target analyte, a competitive agent (in the case of a competitive assay), a tag on the competitive agent (in competitive assay), a secondary antibody (sandwich assay only), a chromogenic substrate, an immobilizing substrate, and a tag on the immobilizing substrate. Some embodiments of the platform 1700 are used to detect cortisol in saliva, but the platform could be used to detect any analyte for which an antibody can be found, and the other reagents would be customized accordingly to the specific analyte of interest. Some embodiments of the platform 1700 use a competitive immunoassay to sense the analyte, but other forms of immunoassay could be used such as a sandwich, direct or indirect assay.

According to some embodiments, a biotinylated rabbit polyclonal anti-cortisol Ab is used, but any animal basis could be used such as a mouse monoclonal, rat, etc. as well as some other conjugate of the above, being bispecific as well. The buffer used to stabilize the antibody is Surmodics Research Assay Diluent in some example embodiments, but any stabilizing buffer for the antibody of choice could be used. For an example assay, which is competitive, the competitive agent is Cortisol-HRP, which is a form of cortisol tagged with the horseradish peroxidase enzyme (HRP). In general, any HRP-tagged analyte could be used, matching the analyte of interest, such as testosterone-HRP for testosterone sensing, etc.; the enzyme could also be different, such as alkaline phosphatase (AP), biotin, or fluorescein. The enzyme used will have a corresponding enzymatic stabilizer, which for the preferred embodiment is HRP stabilizer (StabilZyme™ HRP Conjugate Stabilizer), but any stabilizer for the enzyme can be used. In the preferred embodiment, there is no secondary antibody, but in the sandwich, assay form any antibody from hosts could be used, such as rats, mice, etc. and tagged with HRP, AP, or other tags. The chromogenic substrate in the preferred embodiment is 3,3′,5,5′-tetramethylbenzidine (TMB), but others could be used such as ABTS, AEC, DAB, ECL, and Amplex Red (if using HRP as the enzyme tag); otherwise, PNPP, etc. could be used if AP is used as the tagging enzyme. The immobilizing substrate in the preferred embodiment is a ferromagnetic particle tagged with streptavidin, but other particles could be used such as agarose beads, polystyrene beads, etc.; and the conjugate could be changed to neutravidin, Protein A, Protein G, etc. The buffer used in the washing steps is phosphate buffered saline (PBS) and Tween 20 (Polyoxyethylenesorbitan monolaurate), although other buffer types could be used such as BSS, DPBS, etc. and another detergent could be used, such as Tween 40, Triton X 100, etc.

FIG. 20 is a flowchart of chemical processes within an immunoassay device according to some embodiments. The block diagram on FIG. 19H also shows the process. In Step 2010, saliva is collected, e.g., using collection device 400. When the collection device 400 is reinserted in cartridge base 700, saliva on tip 480 is squeezed off of the tip 480 and passes into the microfluidic system at 1800-S after passing through hole 720H (FIGS. 7F, 7I).

In Step 2020, the saliva sample is magnetically mixed with reagents in reaction chamber 1831 for 7 minutes.

In Step 2030, the reaction chamber 1831 is flushed with air to remove the solution.

In Step 2040, according to some embodiments, the reaction chamber 1831 is washed with a phosphate buffer and/or a detergent and flushed again with air and this process is repeated three times, leaving only the conjugated magnetic particles in the reaction chamber.

In Step 2050, TMB is added to the reaction chamber 1831 with the conjugated sample and incubated with the particles for 5 minutes.

In Step 2060, the sample is pumped to a mixing channel 1833 for homogenous mixing.

In Step 270, after mixing, the substrate is pumped to an electrode 1800-E for analysis. According to some embodiments, the electrode used to quantify the ensuing reaction with TMB is made up of screen-printed carbon ink, any type of carbon ink can be used.

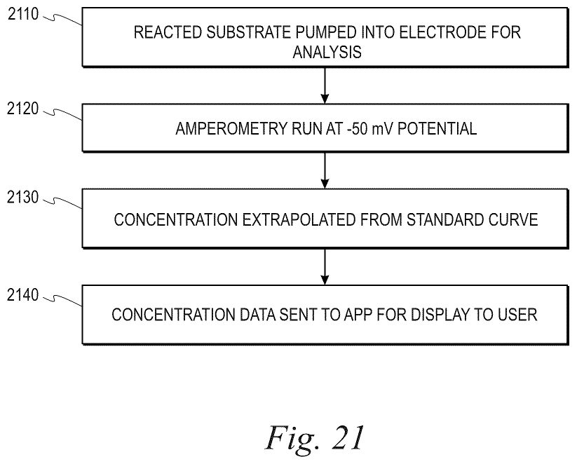

FIG. 21 is a flowchart of chemical processes within an immunoassay device according to some embodiments.

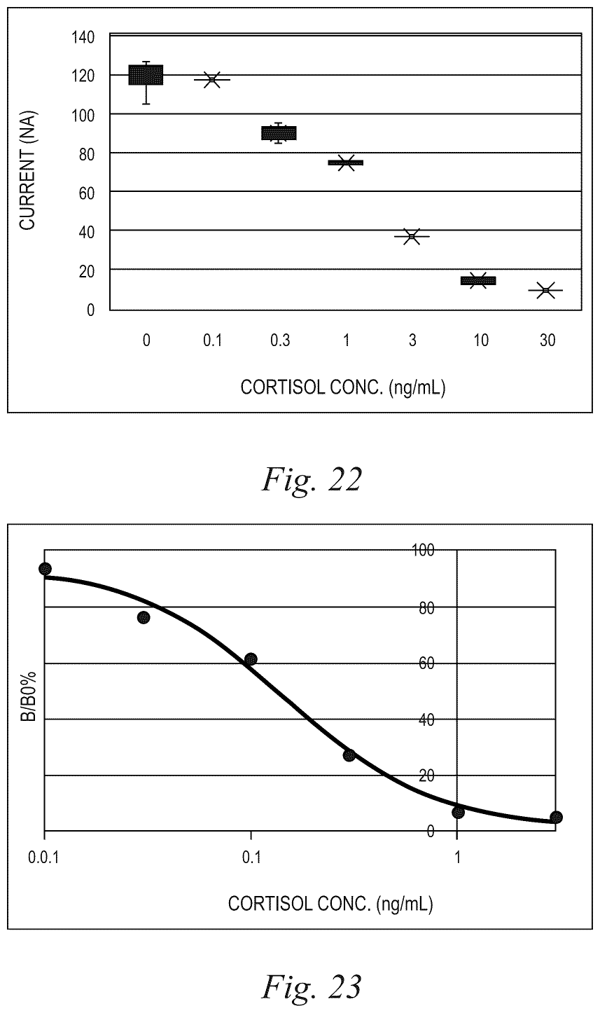

FIG. 21 is an immunoassay processing flowchart according to some embodiments. In Step 2110, reacted substrate pumped to electrode for analysis by internal micropumps. In Step 2120, amperometry is run at −50 mV potential. In Step 2130, a concentration of the analyte (cortisol in the preferred embodiment) is extrapolated from a standard curve (see, e.g., FIGS. 22-23). At Step 2140 the concentration data is sent to an App for display to a user according to some embodiment.

FIG. 22 is a graph of the electrochemical reaction of cortisol according to some embodiments of the present disclosures. FIG. 22 shows the salivary cortisol 4-parameter curve fit for the detection of cortisol in saliva. FIG. 23 is a graph of the sensor response to the different concentrations of cortisol according to some embodiments of the present disclosures. The graph also shows the percent bound (B/Bo) for each saliva sample. B/Bo is obtained by dividing the sensor response of each sample (B) by the average background value for the zero (Bo). The logistic range of cortisol measurement in saliva is between 0.01 and 30 ng/mL, the linear range of measurement is between 0.1 and 10 ng/mL. Cortisol enters saliva by intracellular mechanisms, independently of salivary flow rate. In saliva, most cortisol remains unbound to protein. It reflects the circadian rhythm and early morning peak and responds to changes in plasma cortisol concentration quickly and reliably. Mean salivary cortisol concentrations in the early morning is between 3.6 nmol/L and 8.3 nmol/L and the late-night salivary cortisol value is below 3 nmol/L.

DETECTION METHOD: Chronoamperometry on unmodified screen-printed carbon electrode, which detects the amount of reacted TMB. This amount is inversely proportional to the amount of cortisol (or other analytes) present in the saliva. A standard curve is made using known cortisol concentrations in phosphate buffer solution (PBS) (FIG. 23) which can then be used to correlate current with cortisol concentration. A 4-PL curve fit is used according to some embodiments (Cortisol competitive immunoassay), but other parameterized logistic curves could be used if they fit the data well.

### 6. General

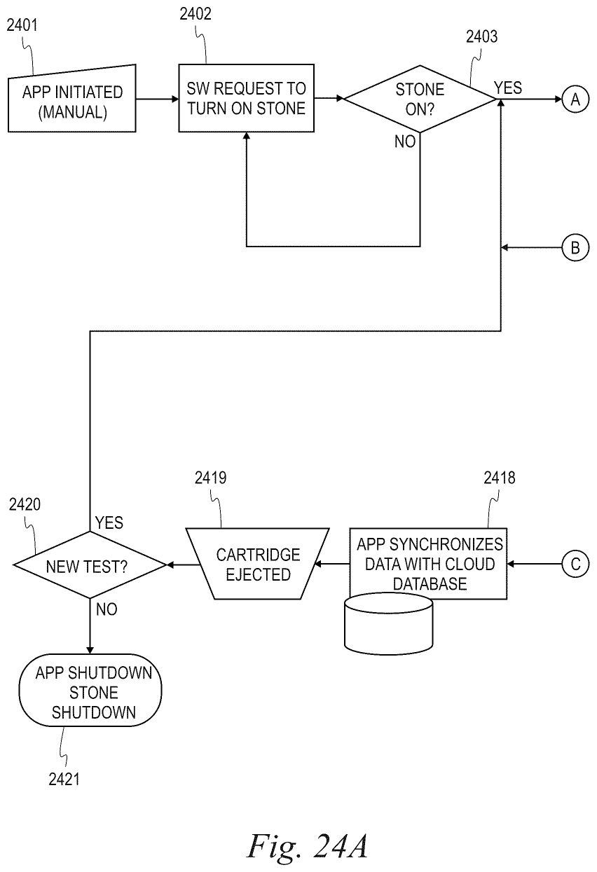

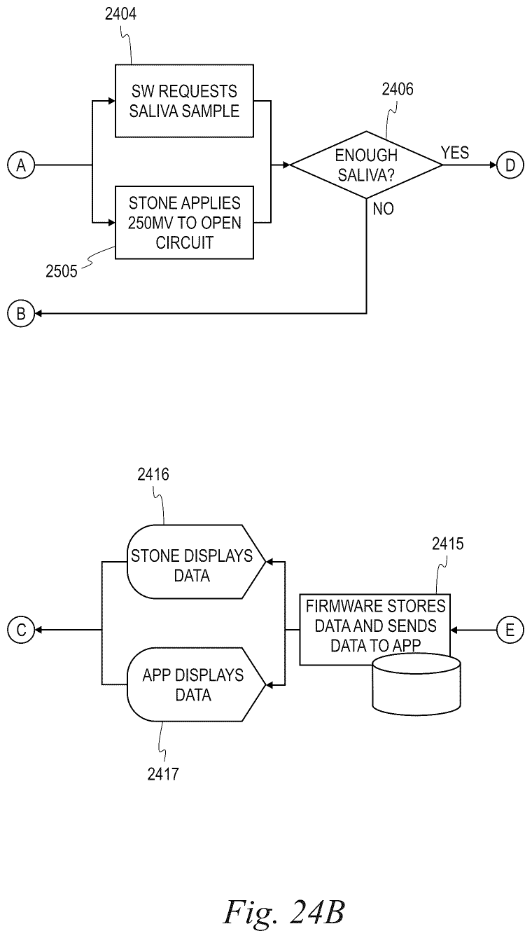

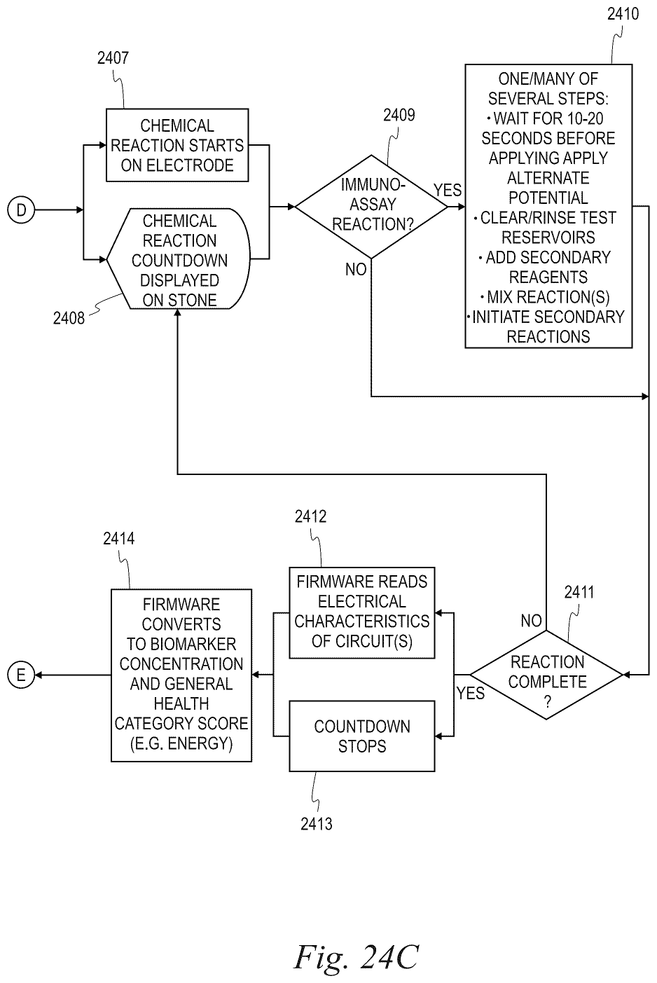

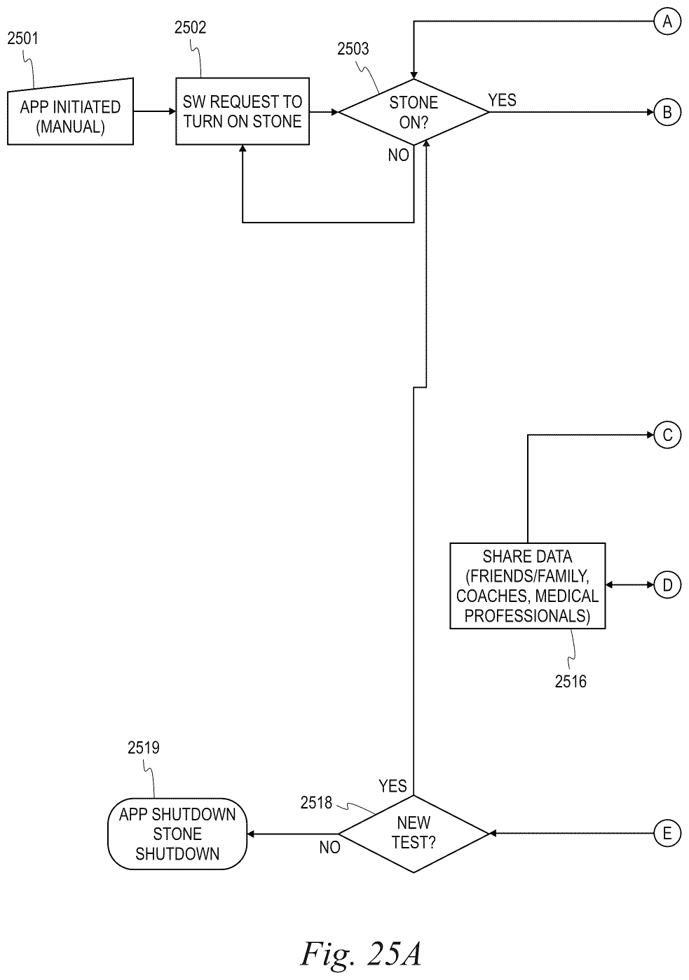

FIG. 24 is a flowchart illustrating a method of testing according to some embodiments of the present disclosure. According to some embodiments, the steps of FIG. 24 may be implemented in firmware and/or software in the POCT device 100. In an example operation:

2401—App initiated (manual). App turned on.

2402—App software requests user to turn on POCT Stone

2403—Stone on? (Yes or No answer). If no, return to Step 2402. If yes, Go to Steps 2404 and 2405.

2404—App SW requests saliva sample.

2405—Stone applies 250 mv in FIG. 15F. The potential is applied to potentiostat 104 and in turn, 160 (the electrical contacts for the sensor in the cartridge) and in turn, 1316, the contacts on sensor 1300.

2406—Enough saliva? (Yes or No answer). If no, return to step Step 2404. If yes, go to Steps 2407 and 2408.

2407—Chemical reaction starts on electrode.

2408—Chemical reaction countdown displayed on Stone.

2409—Immuno-assay Reaction? (Yes or No answer). If no, skip to step 2411. If yes, Go to Step 2410.

2410—One/many of several steps ensure:
Wait for 10-20 seconds before applying apply alternate potential to the sensor 1300 in FIG. 15F, by applying the alternate potential to 160 (the electrical contacts for the sensor in the cartridge) and in turn, 1316, the contacts on sensor 1300.
Clear/rinse test reservoirs
Add secondary reagents
Mix reaction(s)
initiate secondary reactions

2411—Reaction complete? (Yes or No answer). If no, return to Step 2408. If yes, go to Step 2412 and 2413.

2412—Firmware reads electrical characteristics of circuit(s). Specifically, the firmware embedded in the controller 102 in FIG. 15F senses the change in, e.g., current from the potentiostat(s) 104.

2413—Countdown stops

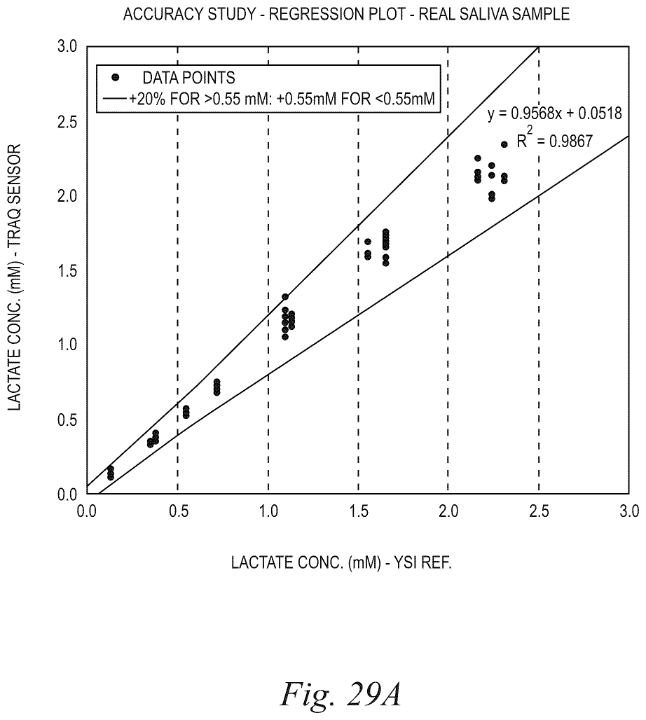

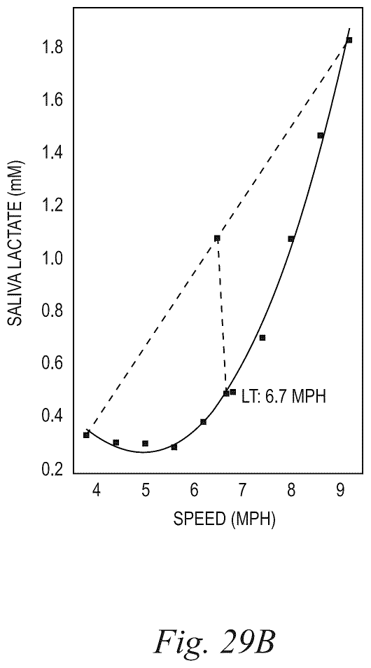

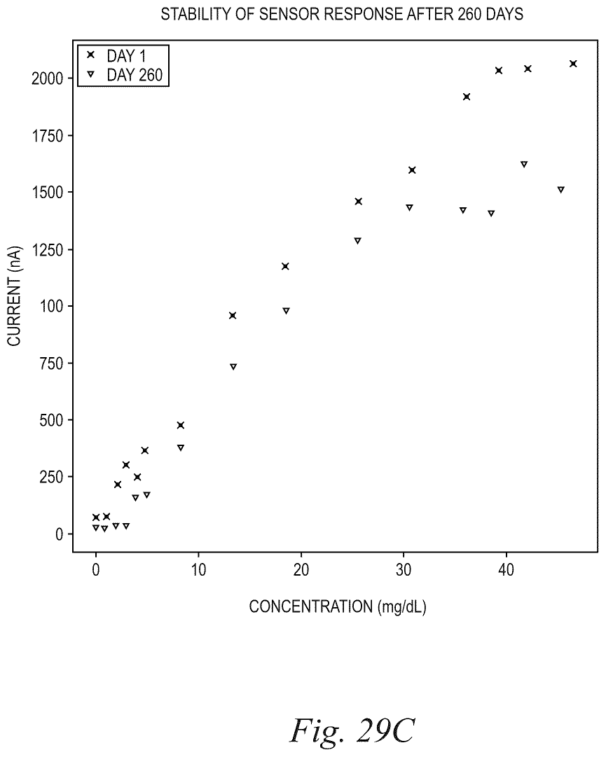

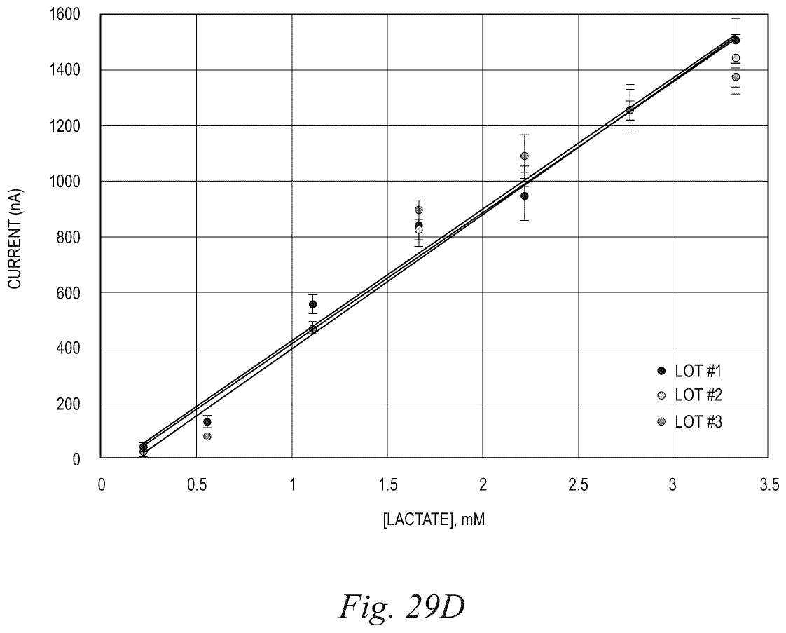

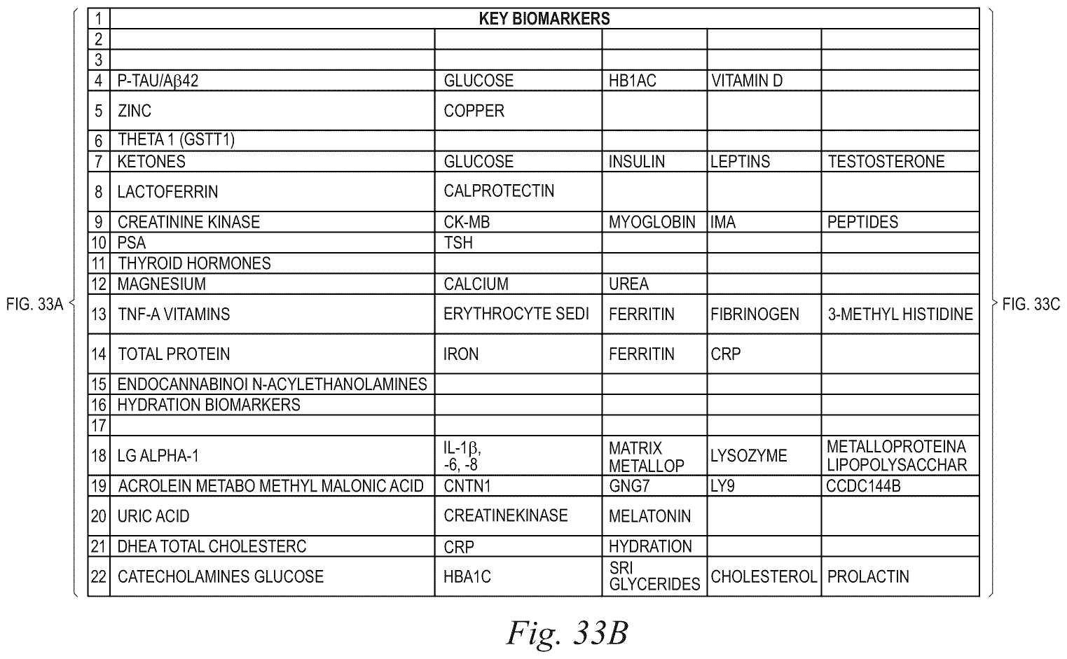

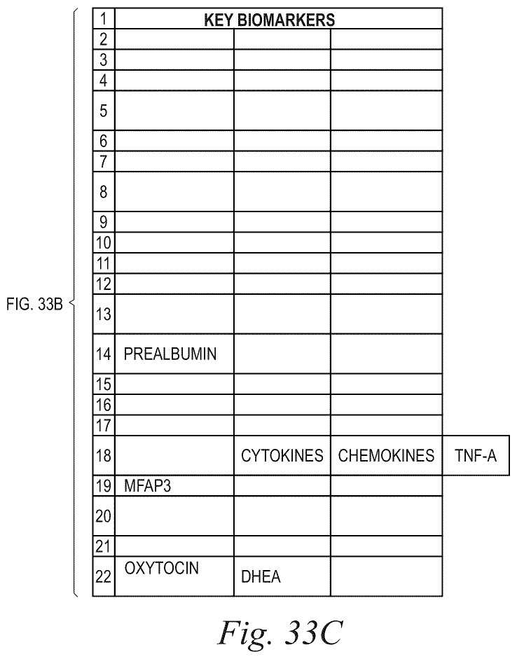

2414—Firmware converts to biomarker concentration and general health category score (e.g. Energy). Specifically, according to some embodiments, the firmware instructs processor 102 to measure the current change on one or more electrodes 1311,1312 via the potentiostat(s) 104. Within the firmware, the current is then converted into biomarker concentration using a known relationship between current and concentration specific to this biomarker, as in example FIG. 29D. Further, this biomarker concentration is also converted into a health score in its related health category, as noted in FIG. 33. For example, a specific lactate concentration reading across electrodes 1311, 1312 and potentiostat 104 might read 800 nA. Using the known relationship between current and lactate concentration in FIG. 29D would yield a lactate concentration of approximately 1.8 nM. This lactate concentration would then relate to an Energy score, per the relationship noted in FIG. 33, line 7, of approximately 50% as it is midway between the normal human range of lactate concentration, where a lactate concentration above 3.4 nM might equal 0% Energy score and a lactate concentration below 0.2 nM might equal an Energy score of 100%.

2415—Firmware stores data and sends data to App

2416—Stone displays data

2417—App displays data

2418—App synchronizes data with cloud database

2419—Cartridge ejected

2420—New test? (Yes or No answer). If no, skip to step 2421. If yes, Go to Step 2404.

2421—App shutdown, stone shutdown

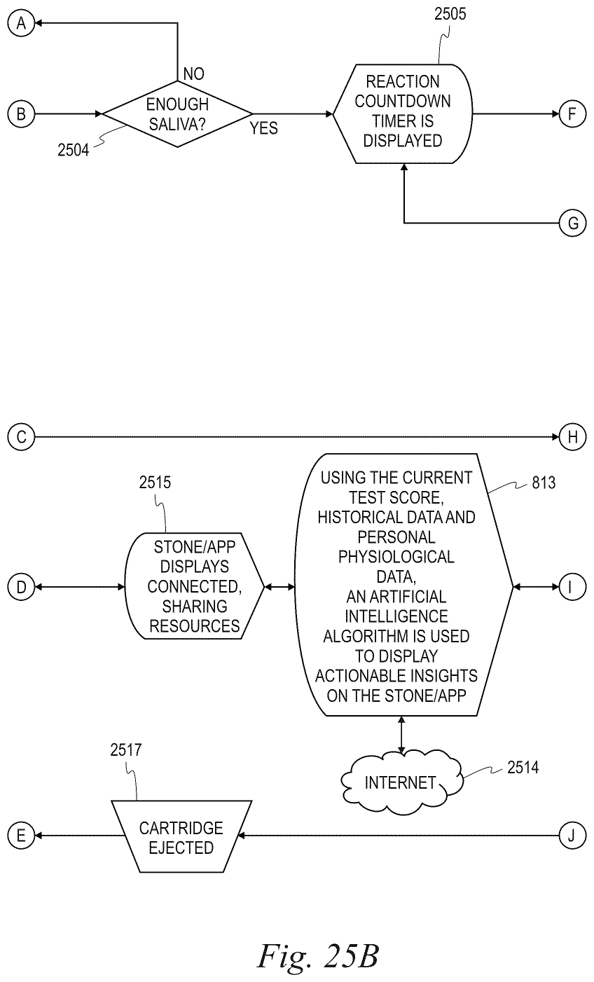

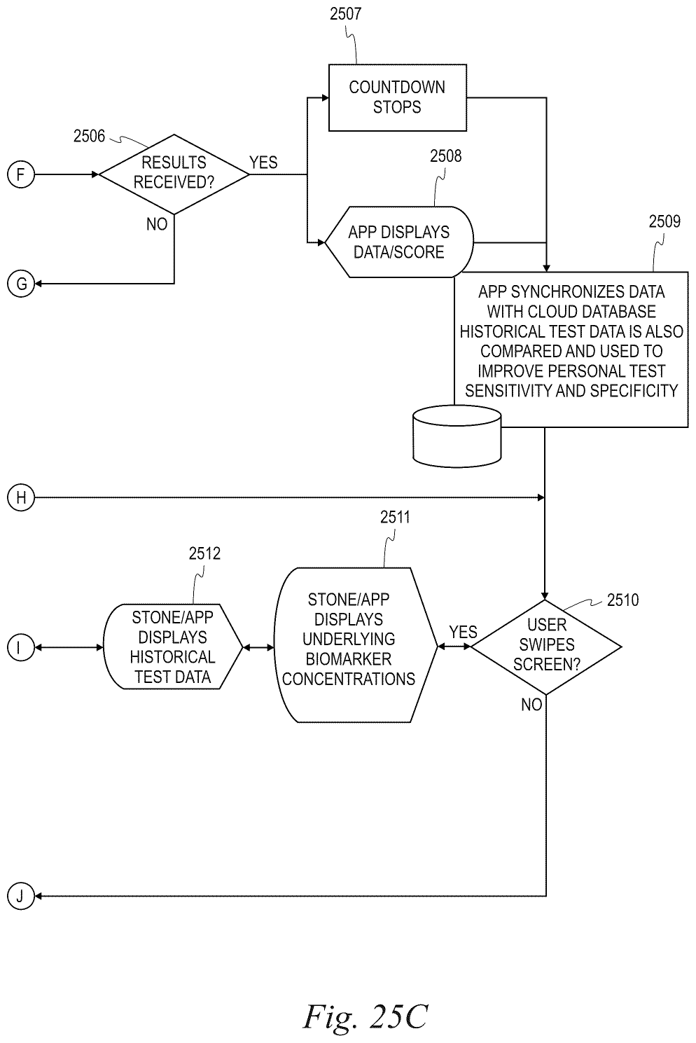

FIG. 25 is a flowchart illustrating a method of testing according to some embodiments of the present disclosure. According to some embodiments, the steps of FIG. 25 may be implemented in software executed by one or more processors in the POCT device 100. In an example operation:

2501—App initiated (manual). App turned on.

2502—App SW requests user to turn on POCT Stone

2503—Stone on? (Yes or No answer). If no, return to Step 2502. If yes, Go to Step 2504.

2504—User collects saliva sample. Enough saliva (Yes or No answer). If no, return to Step 2503. If yes, go to step 2505.

2505—Reaction countdown timer is displayed

2506—Results received (Yes or No answer). If no, return to Step 2505. If yes, Go to Step 2506.

2507—Countdown stops.

2508—App displays data/score.

2509—App synchronizes data with cloud database. Historical test data is also compared and used to improve personal test sensitivity and specificity.

2510—User swipes screen? (Yes or No answer). If no, skip to Step 2517. If yes, Go to Step 2511. Swiping is a physical action a user employs on the screen of their mobile device when operating the App. Swiping the screen to the left or right allows the user to navigate the App and view test results and actionable insights the results might suggest. In this case, if the user swipes the screen, the next report (biomarker concentration) would be displayed.

2511—Stone/App displays underlying biomarker concentrations. Go back to Step 2510 or forward to Step 2512.

2512—Stone/App displays historical test data. Go back to Step 2511 or forward to Step 2513.

2513—Using the current test score, historical data and personal physiological data, an artificial intelligence algorithm is used to display actionable insights on the Stone/App. Go back to Step 2512 or forward to Step 2515.

2514—Internet. According to some embodiments, the current test score and/or other data made by stored in the cloud on the internet and/or the internet is access to retrieve information such as to provide actionable insights.

2515—Stone/App displays connected, sharing resources. Go back to Step 2513 or forward to Step 2516.

2516—Share Data (friends/family, coaches, medical professionals). Go back to Step 2515 or to Step 2510.

2517—Cartridge ejected.

2518—New test? (Yes or No answer). If no, skip to step 2519. If yes, Go to Step 2503.

2519—App shutdown, Stone shutdown.

According to some embodiments, the steps of FIG. 25 may be implemented in software such as in an App on a smartphone or remote device. According to some embodiments, some of the steps are optional and may not be performed and/or may be performed in differing sequences.

According to some embodiments, the POCT system 10 comprises three main components: the handheld Stone POCT device 100, a disposable biofluid or saliva test cartridge 200 and optionally, a mobile or smartphone 191, 2600 or some other remote device (e.g., smartwatch 2620) with an App installed. According to some embodiments, the App is not required for testing but may be required for updating the POCT device 100 and for more involved interaction with the internet and/or for sharing results.

FIGS. 26A-26F and 2611-26P illustrate exemplary displays on a POCT device 100 and/or a remote device such as a smartphone 2600 according to some embodiments of the present disclosure. For the purposes of illustration, both the Stone POCT device 100 and an App on a smartphone 2600 are shown in 26A-26F and 2611-26P. FIG. 26G illustrates the collection of a biofluid such as saliva according some embodiments of the present disclosure. An exemplary embodiment is described below:
1. User Pre-test preparation instructions:
   a. Refrain from eating, drinking, exercising or physical exertion for 90 minutes or more
   b. Gargle with water between 2-5 minutes prior to test.
2. Test.
   a. Activate device (App, POCT)
      i. Turn on TRAQ app on mobile device, computer. You will see the TRAQ Home page. [See FIG. 26A]
      ii. App will notify user to turn on POCT device 100. [See FIG. 26B]
      iii. Turn on POCT 100. [See FIG. 26C]
   b. App may automatically connect to POCT 100 (via Bluetooth) and indicate as such. [See FIG. 26D]
   c. Insert test cartridge and collect saliva sample.
      i. Insert test cartridge into POCT 100. [See FIG. 26E]. App will indicate that cartridge has been inserted and call for user to apply saliva sample. [See FIG. 26F]
      ii. POCT device 100 registers test type and displays (e.g., “Hydration”). [See FIG. 26F]
      iii. User removes biofluid collection device from cartridge base (already inserted in POCT). [See FIG. 26F]
      iv. Collect saliva from mouth. [See FIG. 26G]
      v. When moist throughout the tip (5-20 seconds, usually), reinsert biofluid collection device 400 into cartridge base 700. [See FIG. 26H]
   d. Auto-start test.
      i. According to some embodiments, when saliva is detected by the device 100, the reaction has begun, and the POCT/App show a countdown until the test is complete (usually 30 seconds to 12 minutes). [See FIG. 26I] According to some embodiments, saliva detection may be accomplished by holding a current constant over two of the electrodes, and detecting when there is a resistance change. Detection of the resistance change may cause the processor 102 to trigger the process to start.
   e. Results
      i. When the test is complete, the results/category score will be displayed on the POCT 100 and App, on any one of several devices with the connected App. [See FIG. 26J, 26K]
      ii. For health & wellness, the first screen displayed is a Category score (e.g. Hydration of 63%). For diagnostics, there is no Category score, rather, the key biomarker concentration(s) is(are) shown as in iii) below.
      iii. The user can then decide to drill down to see specific biomarker concentrations (e.g. Sodium, Potassium, etc.) that are used to determine that higher-level health & wellness score (e.g. Hydration) [See FIG. 26L];
      iv. View historical score and test data [See FIG. 26M];
      v. View actionable insights (what does the score mean and what should I do to improve?) or share the results. Link to the internet. [See FIGS. 26N-260]
      vi. View list of sharing resources: friends/family, advisors, coaches or medical professionals and share, contact. [See FIG. 26O]
3. When done, simply eject the cartridge to perform another test or turn off the POCT device 100. [See FIG. 26P]

According to some embodiments, the disposable biomarker test cartridge 200 can be recycled/upcycled. If not already done so, either another disposable biofluid test cartridge 200 may be inserted to conduct another test or the POCT device 100 and App may be turned off [See FIG. 26P]

According to some embodiments, one or more or all of the below advantages may be achieved with the POCT system 10:

Non-invasive: The test uses saliva. No blood draws are required.

No medical professionals are required: Users can test themselves.

On-Demand, Convenient & Private: No appointment need be scheduled. Test anywhere, anytime.

Fast: Results are available in seconds to minutes.

Safer: Disposable cartridges, no blood required, no masking, gloves, etc.

Accurate: Much like sanctioned FDA tests, the test is accurate with 10% (within FDA guidelines)

Economical: Tests cost only a few dollars.

Portable: Handheld or desktop.

Easy: Simple protocol, few steps.

Shareable: Able to share results in near real-time with advisors, coaches or medical professionals.

Single biomarkers are rarely specific enough to give a full picture of one's fitness, health & wellness or medical state. Usually, several underlying biomarkers are required to give a more complete picture and while the some embodiments of device 100 are capable of four simultaneous, unique biomarker measurements, more may be required for an accurate picture and other embodiments may include more than four simultaneous biomarker measurements.

Saliva contamination or dilution is possible, whether by food, drinks (including water), or injury to mouth that allows for blood to enter the mouth. Such contamination would nullify test results.

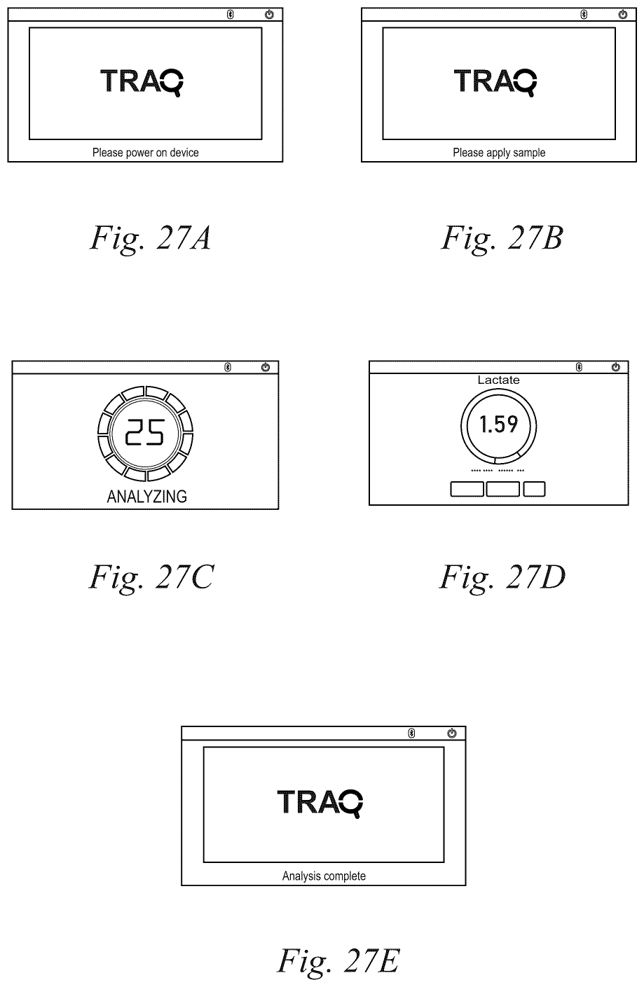

FIGS. 27A-27E are example screen shots of a phone running an application (App) according to some embodiments of the present disclosure.

Example embodiments may provide, among other things, a sample analysis reader configured to be electrically coupled to a sample analysis cartridge, the sample analysis reader comprising: a processor; and a non-transitory computer readable medium having instructions stored thereon, wherein the instructions, when executed by the processor, cause the processor to: detect a presence of the sample analysis cartridge electrically coupled to the sample analysis reader; detect identification information associated with the sample analysis cartridge; identify a proper test protocol for the sample analysis cartridge based at least in part on the identification information; detect a sample collection device inserted into the sample analysis cartridge; and initiate a mixing protocol to mix a sample received form the sample collection device with reagents in fluid in a reservoir in the sample analysis cartridge.

In example embodiments, the processor 102 may be configured to initiate the mixing protocol by pumping reagent and sample from respective reservoirs into a mixing reservoir.

In example embodiments, the processor 102 may be configured to initiate the mixing protocol to facilitate formation of a plurality of competitive complexes between the analyte present in the saliva and the competitive agent form with a streptavidin-tagged magnetic particle.

In example embodiments, the processor 102 may be configured to process a signal from an internal electrode system which is indicative of the quantity of one or more target analytes form the sample

In example embodiments, the sample analysis reader 100 may be configured to be electrically coupled to a plurality of sample analysis cartridges 200 that are each disposable.

Example biosensors as provided herein can provide one or more of the following benefits. However, providing these example benefits is not intended to require that an example biosensor include any one of these benefits. An example biosensor:
Need not rely on batteries or powering in an in vivo manner (in the mouth);
Need not require the device 100 or sensor 1600/1800 to be wearable;
May use easily manufacturable and cost-effective chemicals;
Can be capable of measuring multiple biomarkers/analytes simultaneously;
May have an accuracy well-within FDA requirements for non-salivary biofluids, such as blood-based tests;
May be capable of being employed on any biofluid;
Need not require the use of medical professional to implement the test or read the results;
May employ AI (artificial intelligence) or machine learning to personalize tests for an individual;
Can perform tests in a wide variety or environmental settings without the need of refrigeration or other special storage mechanisms;
Can perform electrochemical tests on multiple classes of reactions: enzymatic, ion selective and/or immunoassay simultaneously;
May use vastly different chemical compounds to perform rapid, sensitive, robust and inexpensive tests;
Can provide a resulting overall categorized health score (Energy, Hydration, Fat Burn, etc.);
May employ a saliva collection device 400 for safe and effective collecting and insertion into POCT testing device 100;
Can enable testing for only a few dollars per test vs. tens or hundreds of dollars per test;
Can remain stable over time (>12 months) without any need for special storage or refrigeration;
Can be capable of interfacing with software for display, storage or for interacting with advisors, coaches or physicians regarding actionable insights to improve overall health, wellness or fitness;
May include associated firmware and software for performing calculations independently; and/or
May be capable of analyzing repeat lactic acid tests for determining lactic acidosis with great specificity and sensitivity, thus serving as an early indicator of several serious maladies.

### 7. Acidosis

According to some embodiments, a point-of-care testing (POCT) system 10 that allows for on-demand, non-invasive (saliva) lactic acidosis screening for severe maladies with results available in seconds, and costing only a few dollars per test, allowing for more frequent testing and thus, earlier detection is provided.

Early detection of disease is desired for both improved patient outcome and cost savings to remedy a medical issue. Some of the most serious and costly diseases that would benefit from earlier detection include, for example:
Cancer (over 20 million new cases worldwide, with over 10 million deaths annually);
Sepsis, causes 1 in 5 deaths worldwide;
Kidney disease, affecting roughly 10% worldwide;
Liver disease, the 11th leading cause of death worldwide; and
Congestive heart issues (affecting 33.5 million people annually).

Current diagnostics for the above medical issues rely on blood tests, scans (MM, PET, Mammogram, etc.,) or specific diagnostic tests (e.g., Pap smear for cervical cancer) along with physical exams by healthcare providers. All these diagnostics procedures help diagnose underlying diseases with great sensitivity and specificity. Blood serum test panels can scan for specific biomarkers that serve as early indicators. Other solutions have included the use of alternative biofluids, such as urine and pH testing strips.

However, each of these procedures or tests are either expensive, time-consuming, subjective in nature, or proscribed either after an issue has already been detected or with a proscribed testing frequency such as annually or some of period. Further, they are usually invasive, cumbersome, or inconvenient and require trained medical professionals. In essence, they don't detect the disease as easily or early as it could be detected with other means.

There is a predictable and measurable relationship between most biomarkers in blood and their counterparts in saliva. Lactate is one such biomarker with a predictable relationship between its concentration in blood serum and saliva.

Lactic acidosis is a sustained, elevated level of lactate that affects millions worldwide each year and is an early indicator of one of several serious, underlying medical issues, including cancer, sepsis, kidney disease, liver disease and/or congestive heart failure, among others.

Specific to lactic acidosis screening, current diagnostic methodologies include the use of blood serum testing in labs to measure lactate levels, requiring invasive blood extraction time for lab processing at an excessive cost, and the presence of a medical professional.

Electronics in the POCT device 100 measure the electrical characteristics of the chemical reaction on the biosensor 1300 within the disposable biofluid or saliva test cartridge 200. The relationship between the electrical characteristics of the chemical reaction and lactate concentration is a known quantity. Thus, according to some embodiments, firmware and/or software in the POCT device 100 converts the electrical characteristics to a lactate concentration when the reaction is complete (e.g., within 30 seconds). In some embodiments, two lactate wells 1331 would be run at the same time, as the saliva coats all five (5) of the wells in every run and averaging the two simultaneous measurements the POCT 100 arrives at a final measurement.

For example, according to some embodiments, two lactate wells 1331 may be read independently and/or simultaneously, and multiple measurements per channel may be averaged and then each average measurement is converted to a corresponding lactate concentration as follows:

a*X_{Avg}²+b*X_{Avg}+c

whereby, the resulting concentration conversion measurements a, b and c might be:
a Value=0.0
b Value=0.0594424
c Value=−72.71592
(time limit(s)=30.0; samples limit: 30; Units=mg/dL)

With a resulting lactate concentration of:

a*X²+b*X+c
where a=0.0, b=0.0594424, and c=−72.71592
and the electrical characteristic from the circuit (voltage, current or resistance) might be XA vg=1250. Therefore:

a*X_{Avg}²+b*X_{Avg}+c

0.0*(1250)²+0.0594424*(1250)−72.71592

=1.59 mg/dl

According to some embodiments, the resulting lactate concentration is:
Displayed on the POCT device 100,
Sent to a mobile app for display, off-device storage, and/or possible sharing of results, and/or
Stored on the POCT device 100 for later retrieval or display.

Once the test is complete, the disposable biofluid or saliva test cartridge 200 may be ejected from the POCT device 100 for disposal or recycling.

According to some embodiments, the entire process usually takes less than 60 seconds, with 5-20 seconds for saliva collection, 5 seconds for collection tip reinsertion into the POCT device 100, and 20-30 seconds to complete the chemical reaction and display/communicate results to the app and/or the display on the POCT device 100. According to some embodiments, the disposable biofluid or saliva test cartridge 200 cost only a few dollars to mass produce.

While lactic acidosis testing is not new, some embodiments of the present disclosure provide and/or contain one or more or all of the following unique features:
non-invasive (saliva) lactate tests;
on-demand testing;
near real-time results;
electrochemical basis for testing;
tests costing only a few dollars;
auto-start testing;
POCT device 100 to app communication;
ability share results in near real-time; and/or
no need for trained medical professionals to conduct test.

According to some embodiments, an expected single use of the system 10 is as described above; however, it would be expected that 2-3 tests over a one-two hour period may be required to confirm results. For example, false high readings may be obtained from: 1) food, biomatter contamination (to mitigate the system 10 may instruct a user to gargle with water within 5 minutes of testing, but no sooner than 2 minutes prior to testing, and such action may be part of the protocol to ensure that the saliva sample is uncontaminated either by food/biomatter or diluted by water), 2) recent physical activity or exercise can inflate lactate readings (testing protocol calls for testing no sooner than within 90 minutes of physical exertion or exercise).

After multiple tests, if elevated, sustained levels of lactate are detected, counsel can be provided to the subject for follow-on services or testing. In this manner, subjects can easily, non-invasively, privately, rapidly and inexpensively test for lactic acidosis.

While some embodiments describe a disposable biofluid or saliva test cartridge 200; however, other embodiments may contain chemistry that allows for multi-use (multi-testing) or be non-disposable. According to some embodiments, within the POCT device 100, two measurements are made of a single sample which are then averaged. Other embodiments may use a single measurement or more than two. Further, it may be possible to employ pre-existing portable devices (such as a mobile phone) to do the calculations that are currently done on the stone, thus eliminating the need for that separate stone device.

According to some embodiments, systems and/or devices are provided that relate to a saliva sensor, a biosensor, a salivary biomarker, a handheld medical device, lactic acidosis testing, cancer screening, sepsis screening, kidney disease screening, live disease screening, congestive heart failure screening, and/or diabetes screening.

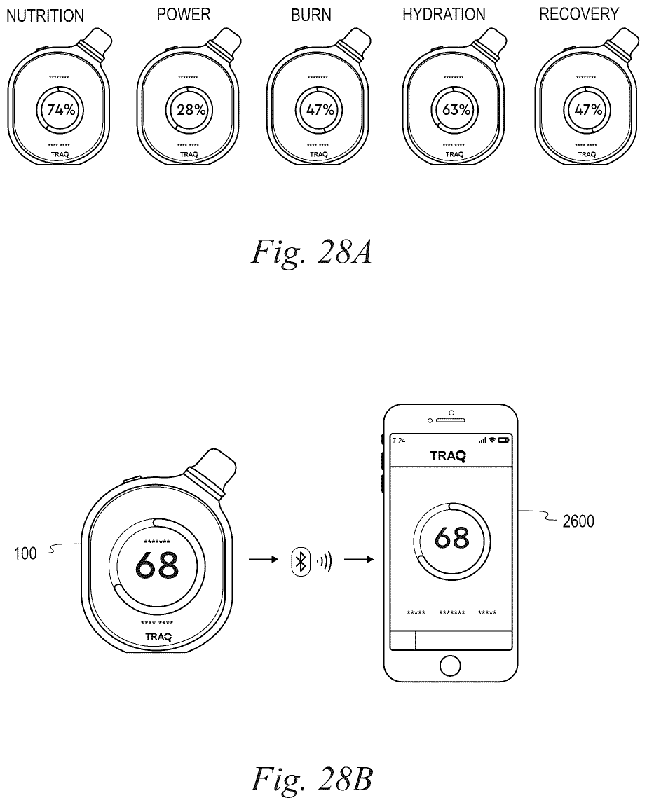

According to some embodiments, lactose testing using the system 10 may entail the following parameters.
1. Pre-Test Preparation:
   a. Refrain from exercising of physical exertion for 90 minutes or more
   b. Gargle with water between 2-5 minutes prior to test.
2. Test.
   a. Activate device (App, POCT 100)
      i. Turn on TRAQ app on mobile device, computer. App will notify user to turn on POCT device 100. FIG. 26B shows an exemplary screen shot of the App instructing a user to turn on the POCT device 100 according to some embodiments of the present disclosure.
      ii. Turn on POCT device 100. App will automatically connect to POCT device 100 (via Bluetooth) and indicate as such.
   b. Insert disposable biofluid or saliva test cartridge 200 and collect saliva sample.
      i. Insert disposable biofluid or saliva test cartridge 200 into POCT 100. App will indicate that cartridge has been inserted and call for user to apply saliva sample. See, e.g., exemplary App screen shown in FIG. 26F.
      ii. Remove collection tip 400 from cartridge base 700 (already inserted in POCT 100).
      iii. Collect saliva from mouth.
      iv. When moist throughout the tip (5-20 seconds, usually), reinsert collection tip into cartridge base.
   c. Auto-start test.
      i. When saliva is detected by the device, the reaction has begun, and the POCT/App show a countdown until the test is complete (usually 30 seconds or less). See, e.g., exemplary App screen shown in FIG. 26I.
   d. Results
      i. When the test is complete, the results will be displayed on the POCT and App. See, e.g., exemplary App screens shown in FIGS. 28A-28B.
      ii. The user can then decide to view actionable insights (what does the score mean and what should I do?), share the results, or simply turn off the POCT device 100 and App.
3. Eject the cartridge for disposal or recycling.

If not already done so, either another test cartridge is inserted to conduct another test or the POCT device 100 and App may be turned off.

According to some embodiments, a system 10 is provided that has one or more or all of the following advantages:
Non-invasive: The test uses saliva. No blood draws are required;
No medical professionals are required: Users can test themselves;
Convenient & Private: No appointment need be scheduled. Test anywhere, anytime;
Fast: Results are available in seconds to minutes;
Safer: disposable biofluid or saliva test cartridge 200, no blood required, no masking, gloves, etc.;
Accurate: Much like sanctioned lactic acidosis tests, the test is accurate with 10% (within FDA guidelines);
Economical: Tests cost only a few dollars.
Portable: Handheld or tabletop (ie. Not handheld but will sit on the user's table/counter like a toaster); and/or
Easy: Simple protocol, few steps.

With these advantages, more frequent testing is made possible, thus earlier detection is possible, saving more lives and money.

Lactic Acidosis tests in general (both blood and saliva) are non-specific as a marker for cancer or any of the other maladies noted above. Rather, the test indicates a general health problem that is best further diagnosed through existing tests and procedures also noted above. This is true of both our solution and other solutions involving lactic acidosis testing.

Lactic acidosis testing in general (both existing solutions and ours) my give false reading if exercise or significant physical activity has been undertaken within 30-90 minutes of the test.

According to some embodiments, saliva contamination or dilution is possible, whether by food, drinks (including water), or injury to mouth that allows for blood to enter the mouth. Such contamination may nullify test results.

FIG. 29A is a graph of an accuracy study from a POCT sensor 1300 configured to measure lactate, according to some embodiments of the present disclosure, as compared to an industry standard lactate concentration analyzer from YSI (YSI benchtop analyzer). Statistical analysis of the accuracy of the sensor 1300 includes primary analysis and secondary analysis. The saliva samples were collected, and the salivary lactate values were measured using a reader/POCT device 100 and a YSI 2300 Stat Plus (YSI Incorporated, Yellow Springs, OH). YSI was selected as the reference instrument and each YSI salivary lactate values were paired with the corresponding POCT sensor values. The correlation analysis of the POCT sensor readings and YSI values revealed that there was a significant positive correlation between them (R²=0.98678, P<0.001). Clarke Error Grid plot was constructed using the salivary lactate values which is shown in the figure. The scatter plot was created based on the YSI values (horizontal coordinate) and POCT sensor readings (vertical coordinate), and the Clarke error grid was constructed with ±20% for ≥0.55 mM and ±0.055 mM for <0.55 mM. Clarke Error Grid Analysis indicated that all the readings fell in the clinically acceptable zones.

FIG. 29B is a graph showing the salivary lactate response to incremental physical exercise according to some embodiments of this disclosure. The testing protocol involved an incremental exercise test for consecutive periods of 3-minute durations, interrupted by a resting period of 1 minute, during which the saliva samples were collected, lactate values were estimated. Typically, the test began at a fixed pace of 3.8 mph with a progressively increasing speed of 0.6 mph for every 3 minutes. This exercise/rest cycle was continued until the subject reached their endurance peak (volitional exhaustion). Salivary lactate profiles constructed using salivary lactate values show similar response to blood lactate profiles during the incremental exercise test. In all the cases, a clear inflection point in the lactate profile was observed with increasing intensity. The rate of change in the saliva lactate with increasing workloads was proportional to that observed in the blood.

FIG. 29C is a graph of a stability study by plotting comparing measurements obtained using a POCT sensor 1300 configured to measure lactate on Day 1 when the sensor is manufactured, up to Day 260 after the sensor was manufactured according to some embodiments. The real-time shelf life of the POCT sensors 1300 was evaluated by showing the sensor response to different concentrations of lactate on Day 1 and Day 260. The POCT lactate sensors were stored at room temperature over silica gel as desiccant in the storage vial for various periods of time and the sensor 1300 response to the different concentration of salivary lactate (mg/dL) was measured at various time intervals. The vials were stored closed throughout the study period and stored at room temperature. The results show that the POCT sensor 1300 response is comparable after 260 days of storage.

FIG. 29D is a graph showing lot-to-lot manufacturing variability and robustness by plotting measured biomarker concentrations in three different production lots and applied to biosensor chips 1300 according to some embodiments. Manufacturing lot-to-lot variation may be defined as a change in the analytical performance of a POCT sensor 1300 from one production lot to the next. The POCT sensors 1300 from three different lots were selected and the sensor response to the different concentration of lactate was studied. The study was conducted within the analytical range of the lactate assay. Testing of each sample was carried out on the same day, using the same lactate control solutions and the same operator. No statistically significant variability between lot-to-lot manufacturing was observed.

According to some such embodiments, the biosensor 1300 consists of a screen-printed electrode 1311 within physical depressions or wells 1331 which contain enzymatic chemistry for detecting lactate in saliva (or other biofluids), along with electrical connection points 1316, for interaction with the handheld measuring tool 100.

According to some such embodiments, collectively the system 10 makes an electrochemical measurement, whereby the electrical characteristics of a chemical reaction are measurable and are directly related to lactate concentration in the biofluid. According to some embodiments, a user inserts the entire test cartridge 200 (saliva collection device 400, base 700 with embedded biosensor 1300) into the POCT device 100, and then removes the saliva collection device 400 from the cartridge base 700 to collect a sample from their mouth. Once saliva has been collected by the saliva collection device 400, the user would then re-insert the moist collection device 400 into the cartridge base 700. According to some embodiments, the system automatically senses a chemical reaction between the saliva sample and the biosensor 1300.

Specifically, according to some embodiments, the biosensor 1300 works by reading the electrons added to the working electrode by the mediator during a redox reaction. Specifically, according to some embodiments, chronoamperometry is used between a reference electrode 1323 and a working electrode 1322. According to some embodiments, three data points are measured and then averaged in the beginning, middle and near the end of the chronoamperometry run. The current average value is converted to a potential and then to a corresponding concentration of measured analyte.

According to some embodiments, the biosensor 1300 is configured to measure lactate and its chemistry consists of the following ingredients:
50 mM HEPES, 50 mM BTP, DI water, Polyvinylpyrrolidone, hydroxyethyl cellulose, 0.9% NaCl, 5% Triton X, Methocel, Trehalose, Sodium Succinate, Ruthenium Hexamine, and lactate oxidase.

The working electrode 1322 material has a redox mediator and a lactate oxidase (LOx) thereon. The LOx may be from any source such as microorganism (e.g., toyobo, LCO 301) e.g., Aerococcus viridans. The electrode 1322 material and an analyte-related enzyme form an enzyme mix capable of catalyzing a reaction. The redox mediator is capable of transferring electrons between the enzyme-catalyzed reactions and the working electrode 1322. The reference electrode 1323 serves as a voltage reference between the solution and the working electrode 1322, allowing for the correct voltage difference to be read from the working electrode.

The following formula is related to the above reaction:

L-Lactate+O₂(LOx) [symbol] Pyruvate+H₂O₂

According to some embodiments, as part of the lactate biosensor 1300, the reagent preferably contains two enzymes, a reduced form of a redox mediator, at least one binder, and a surfactant. According to some embodiments, the enzymes are glucose oxidase (GOD) and the peroxidase mentioned above.

### 8. Health Categories

According to some embodiments, a system 10 and/or device 100 relates to health & wellness, biomarkers, health measurement and scoring, artificial intelligence, and/or diagnostics. An example scoring method that can be performed in part by or entirely by an example system can include the following:
Delineate the various biomarkers that are known to be related to a general category of fitness, health or wellness. These are called the underlying biomarkers.
Assign (or receive or retrieve (e.g., from memory or storage) an already-assigned) a weighted importance factor (numeric) to each underlying biomarker as it relates to the optimal level of that category in terms of fitness, health or wellness. That is, one underlying biomarker may have more importance or significance than another. This relative importance score is assigned, for example, as percentage with the combined importance factors of all underlying biomarkers totaling 100, as a percentage.
A weighted importance factor may be derived from any appropriate source, such as by not limited to historical scores, the broader population or physiological parameters of the user (age, gender, weight, height, etc.) or some combination thereof, e.g., via a suitable algorithm, e.g., an artificial intelligence algorithm.
Assign a concentration score, from 0 to 100, to each biomarker that is related to its measured concentration within the biofluid of the subject population, with healthier concentrations figures receiving concentration scores closer to 100 and unhealthy concentrations receiving concentration scores closer to 0.
Combine the weighted importance score and concentration score for each underlying biomarker.
Total these combined underlying biomarker scores to calculate or derive a final score or numeric for that specific fitness, health or wellness category.

The example salivary point-of-care biomarker measuring device can be configured to measure any one of thousands of biomarkers, and the measurement can be used to generate an overall category health score, displaying results on the device and communicating results to software application.

An example software application can communicate with the POCT device, displaying overall health category scores(s), underlying biomarker concentrations, self-directed actionable insights to aid the user in improving their overall health category score and links to advisors, coaches, health professionals or others for external insights.

Current fitness, health and wellness scoring systems fail to measure specific chemical biomarkers related to those areas of fitness, health and wellness. Instead, various systems limit themselves to:
Activity tracking—steps, miles, calories, weight lifted, etc. Quantifiable, but not liked to a specific biomarker, nor to a general category of fitness, health and wellness.
Interviews for scoring non-chemical biomarkers, such as steps, heart rate, general feelings. Quantifiable, not linked to actual physiological metrics.
Linking specific maladies to a specific biomarker concentration, such as Diabetes and blood serum concentration of Glucose or Insulin. Quantifiable, but not linked to general categories for fitness, health and wellness.
Expressing fitness, health and wellness categories as relative qualities: good, bad, OK, fine, great, poor, so so, etc. Not numeric. Hard to compare.

No existing system attempts to quantify (e.g., score) general fitness, health and wellness categories using related, underlying chemical biomarkers, and in a numerical fashion. With quantifiable scores, users can more easily compare fitness levels, health and wellness categories, among a larger population, or against their own improvement or lack of improvement. Further, in non-human populations, a scoring system is invaluable in understanding subject's fitness, health and wellness, given the likely lack of communication. A scoring system for the measuring the “Immunity” level or “Hydration” level of a pet or livestock, would prove very valuable.

Further, with quantified metrics, more precise actions (actionable insights) can be taken in relation to a poor or good score.

The value of the system 10 according to some embodiments relies on accurately assigning underlying biomarkers to a category. Typically, there is no single biomarker that is indicative of neither a disease, nor a broad category, such as mental health. There usually are multiple underlying biomarkers affecting a general fitness, health or wellness category.

According to some embodiments, the value of the system 10 also relies on accurately measuring biomarker concentrations. Currently, blood is considered the “gold standard” as it rates to biomarker concentrations, as doctor visits usually start with collecting vitals and likely blood samples for testing. Using other biofluids, that are less consistent or reliable, can also cloud the scoring results.

Lastly, some subjectivity will remain as long as the numeric definitions of a “normal”, “healthy”, “unhealthy” and “abnormal” biomarker concentration exist.

Some embodiments of the disclosure provide a system that quantitatively defines typically non-quantified health, fitness and diagnostic health measures using the concentration of related underlying biomarkers within saliva and/or other biofluids (blood, urine, tears, sweat), and a user's general physical parameters, comprising;
(a) Concentration(s) of various biomarkers related to a qualitative health category generally associated the underlying biofluid;
(b) Weighting factors assigned to each biomarker as it relates to the overall importance to the qualitative assessment within a health category;
(c) An algorithm that combines the concentrations and weighting factors in one of several fashions to compute a final health category score;
(d) where the final health score may be a scaled numeric (e.g. 1-10, or 1-100), binary (e.g. high-low), or any other stratified scale that denotes a quantitative difference between poor health and excellent health (e.g. high-medium-low, green-yellow-red);

An example intelligent algorithm may use a score along with pertinent user health information, to modify weighting factors for a personalized weighting schedule;

An example system may additionally determine and provide to the user actionable insights related to the health category score that provide the user recommendations on how to improve the overall health category score.

The POCT device or other device in the system can provide an on-device display of underlying biomarker concentrations, health category score and self-directed or external actionable insights that relate to health category score improvement;

A mobile software application may be provided that displays underlying biomarker concentrations, health category score and actionable insights.

Some embodiments of the disclosure provide a device with a microprocessor and sensing elements capable of measuring biomarker concentrations, storing and altering weighted factors, manually or based on an artificial intelligence algorithm, and calculating a final health category score;

The device may further comprise means for communicating with and transmitting the biomarker concentrations, weight factors and overall health category scores back and forth between the measuring device and software application(s) and/or databases.

The device may further comprise means for of sharing quantitative health category scores with coaches, medical advisors or others, for further actionable insights or counsel.

### Health Categories:

A(some) biomarker(s) serve as underlying fitness category indicators, such as lactate as an indicator of Energy levels. Other categories might include Nutrition or Hydration. Yet other biomarker(s) indicate general health & wellness, such as Hearth Health or Cognitive health. Further, some of these same biomarkers, or others, serve as indicators of a specific medical condition. In general, these salivary biomarkers can help one determine their fitness, general health & wellness and/or a diagnostic condition in an on-demand, rapid, private, cost-effective manner.

Once a biomarker(s) is/are measured:
A fitness, health & wellness or a diagnostic condition score may be displayed on the POCT device 100 (and/or elsewhere);
The concertation of the underlying biomarker(s) may be displayed on the POCT device 100 (and/or elsewhere);
Results may be sent to a mobile app for display, off-device storage, providing actionable insights related to the overall score or biomarker concentrations, and possible sharing of results;
Results may be stored on the POCT device 100 (and/or elsewhere) for later retrieval or display.

Once a salivary test is complete, the associated cartridge may be ejected from the POCT device 100 for disposal or up/recycling.

According to some embodiments, the entire process usually takes between 30 seconds to 12 minutes, with 5-20 seconds for saliva collection, 5 seconds for collection tip reinsertion into the POCT 100, and 20 seconds to 11 minutes to complete the chemical reaction and display/communicate results, e.g., to a related app. According to some embodiments, cartridges 200 cost only a few dollars to mass produce.

According to some embodiments, once testing is complete and results displayed, the subject, e.g., user, can delve further into their fitness, health & wellness or medical condition through “actionable insights” available on the POCT device 100 and/or a companion App. According to some embodiments, the actionable insights may allow the user to learn more about their state and start to act. Further, according to some embodiments, the subject, e.g., user, may be then linked to various coaches, advisors or healthcare professionals for an easier entry-point into the healthcare system. According to some such embodiments, the stone 100 connects to another device (e.g., a coach's smartphone) such as via Bluetooth. In “coach mode” the stone 100 can contain all the data for individual team members and send them to the cloud or a master smartphone so that a coach may have a record of, e.g., lactate threshold increase, etc. for each athlete on the coach's team.

According to some embodiments, cartridges 200 can be biomarker specific (e.g., lactate, glucose, cholesterol) or fit into anyone of several fitness categories (e.g., Energy, Stress, Hydration, etc.)

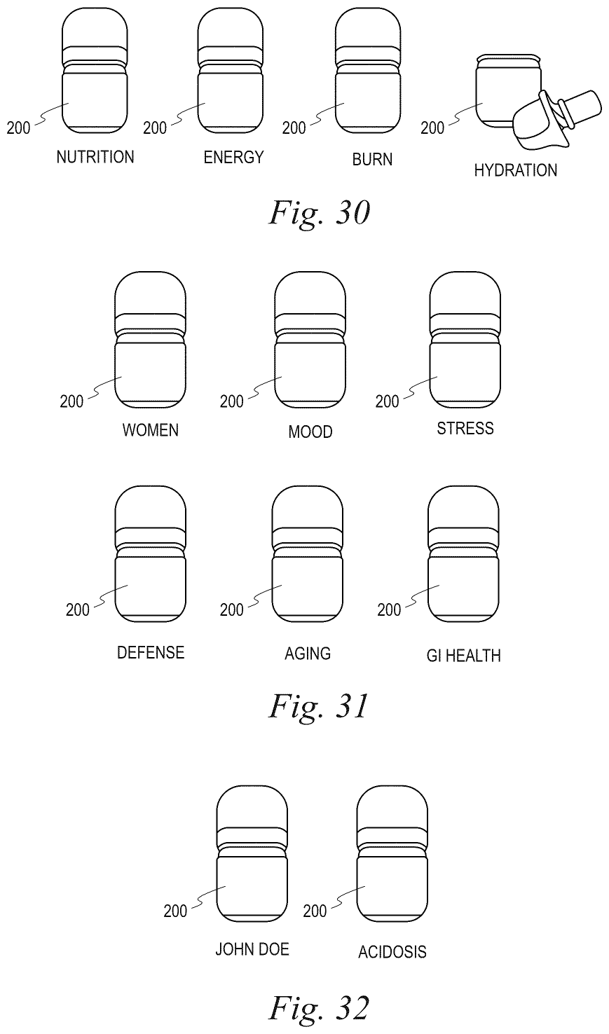

FIG. 30 illustrates sample disposable biofluid or saliva test cartridges 200 for various potential fitness categories (Nutrition, Energy, Burn, Hydration), etc.) of the present disclosure.

FIG. 31 illustrates sample disposable biofluid or saliva test cartridges 200 for health & wellness categories (e.g., aging, defense, mood, GI Health, stress) or gender-based cartridges (e.g. women's' health) of the present disclosure.

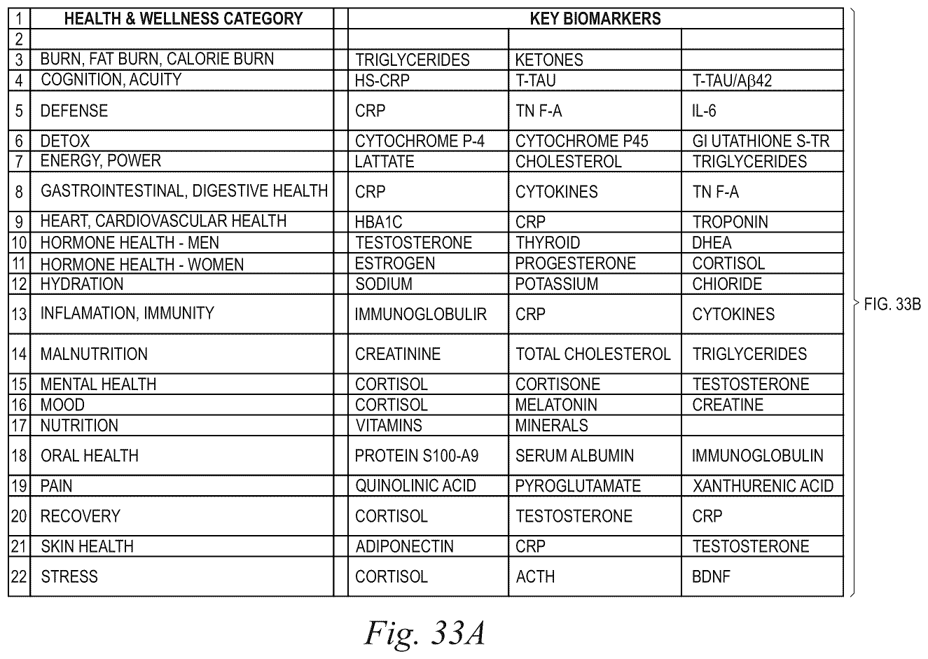

FIG. 32 illustrates sample disposable biofluid or saliva test cartridge 200 for specific maladies or medical conditions (e.g., acidosis) or personalized medicine with dedicated cartridges for an individual (e.g., “John Doe”) according to some embodiments of the present disclosure.

According to some embodiments, one or more or all of the below features are provided:
non-invasive (saliva) tests for specific biomarkers, fitness levels, health & wellness categories, or medical conditions;
on-demand testing, wherever, whenever for complete control and privacy;
portable testing with handheld or small desktop apparatus;
testing one/more salivary biomarkers simultaneously;
simple, as no medical personal required to administer, read or share tests;
near real-time results;
safe, with disposable cartridges;
accurate salivary testing to within current FDA requirements;
electrochemical basis for testing;
tests costing only a few dollars;
auto-start testing;
POCT device 100 to app communication; and/or
ability share results in near real-time

An expected single use of the device is as described above. However, according to some embodiments, it may be expected that several tests over some period may be required to confirm one or more results as false high readings that may be obtained from:
1) recent ingestion of food or liquids—much like blood tests, according to some embodiments, subjects may be required to refrain from eating up to 2 hours before such tests;
2) food, biomatter contamination—according to some embodiments, the testing protocol may require subjects to gargle with water within 5 minutes of testing, but no sooner than 2 minutes prior to testing, to ensure that the saliva sample is uncontaminated either by food/biomatter or diluted by water;
3) stress, or recent physical activity or exercise can inflate or depress certain biomarker readings—according to some embodiments, the testing protocol may call for testing no sooner than within 90 minutes of physical exertion or exercise or stress-related event.

Some embodiments describe single-use, disposable biofluid or saliva test cartridge 200; however, other embodiments may contain chemistry that allows for multi-use (multi-testing) or be non-disposable.

According to some embodiments, within the POCT system 10, two measurements are made of a single saliva sample which are then averaged. Other embodiments may use a single measurement or more than two. According to some embodiments, up to four salivary biomarkers may be measured simultaneously, however other embodiments may envision fewer or more biomarkers being tested simultaneously. Further, it may be possible to employ pre-existing portable devices (such as a smart phone) to serve as the POCT device 100 itself, thus eliminating the dedicated POCT device 100. According to some such embodiments, the functions of the stone 100 (computation, analysis) can be split between the smart phone and the disposable biofluid or saliva test cartridge 200, respectively. Other embodiments may not even envision human health and human saliva, rather other animals or mammals, such as pets or livestock. Lastly, biofluids other than saliva might be imagined.

According to some embodiments, health & wellness category definitions and a quantitative scoring system are provided.

General health statements (e.g., energy level, dehydration, ability to recover, etc.) provide high level information regarding one's health, however, there is no direct connection to specific, underlying physiological biomarkers used to define those broad health categories. Further, there is no quantifiable (numeric) metric assigned to these broad categories for the purposes of comparison. For example, there is no numerical score to define “Energy.” This makes it very difficult to compare against historic Energy “feelings” or with other Energy feelings of other individuals.

Current solutions to this problem do link specific maladies to underlying biomarkers. For example, diabetes is typically defined by the concentration of glucose and insulin (as well as dozens more biomarkers with less sensitivity or specificity for the disease).

Despite the direct connection of specific maladies to specific biomarkers, there still is a lack of connection between higher level general health & wellness categories and specific underlying biomarkers, as well as a lack of assignment of the relative importance of those various underlying biomarkers to the health category in question. Further, there is no existing system for assigning numeric values, or scoring, to the broad health & wellness categories in an attempt to quantify these categories and make actionable corrections to improve one's score or compare populations.

Several general health & wellness categories, known both in the medical world as well as to laypeople, exist. However, when an individual states that they have “good energy,” are in a “good mood,” or feel “dehydrated,” what does that mean on a physiological level?

According to some embodiments, through direct research, clinical trials and research review of published clinical papers, various primary chemical biomarkers are delineated that indicate these general health categories.

First, the following Health & Wellness categories were defined (these are not exhaustive):
Burn, Fat Burn, Calorie Burn
Cognition, Acuity
Defense
Detox
Energy, Power
Gastrointestinal, Digestive health, digestion
Heart, Cardiovascular Health
Hormone Health—Men
Hormone Health—Women
Hydration
Inflammation, immunity
Malnutrition
Mental Health
Mood
Nutrition
Oral health
Pain
Recovery
Skin Health
Stress

Next, various underlying chemical biomarkers were then defined as broadly impacting those health & wellness categories.

FIGS. 33A-33C illustrate a table of examples of Health Categories & exemplary related, underlying biomarkers according to some embodiments of the present disclosure. The Health Categories can be defined in more detail by the concentrations of the listed, related and underlying biomarkers. Each noted biomarker holds a varying degree of importance as an indicator of that health & wellness category. Some biomarkers listed hold more importance while other are less important. The list is not exhaustive as in some cases, dozens or hundreds of individual biomarkers may have an impact on a general Health Category, albeit in varied levels of importance.

With reference to FIGS. 33A-33C, each noted biomarker holds a varying degree of importance as an indicator of a corresponding health & wellness category. Some biomarkers listed hold more importance while other are less important.

Finally, according to some embodiments, a system was developed to assign a numeric score to a general Health & Wellness category according to: 1) the relative importance of the biomarker as an indicator of the health & wellness category in question, and 2) the relative concentration of the biomarker in the relative biofluid (blood, saliva, urine, sweat, tears, etc.), 3) along with the physiological state of the user with such information as gender, health, weight, age, etc.

To employ this health scoring system, each related biomarker was assigned a score of importance (IS) as it relates to the higher-level health category, from 1-100. For a biomarker with more importance, its IS is closer to 100. For a biomarker with less importance as an indicator within that category, its IS is closer to 0. Collectively however, all biomarkers' IS scores related to a particular Health & Wellness category add up to 100.

FIG. 28A shows various devices 100 associated with different tests or general health categories and associated scores are displayed on the display of the device 100 and provides examples of qualitative health categories with sample related underlying biomarkers according to some embodiments of the present disclosure.

For example, for the broad measure of “Energy” the following biomarkers were found to be of indicative value: Lactate, Glucose, Testosterone, Cholesterol, Triglycerides, Ketones, Insulin, Leptins. The Energy IS scores were assigned as follows:
Lactate: 60
Glucose: 30
Testosterone: 4
Cholesterol: 2
Triglycerides: 1
Ketones: 1
Insulin: 1
Leptins: 1
Total IS: 100

The individual IS assignments may fluctuate due to both subjective and factual input. For example, it may be found that there are several more biomarkers that have impact on the Energy category, that one biomarker has more or less relative importance than noted here or that the assigner of the IS may have a bias of one biomarker over another.

If a biomarker is not measured, it is removed from the IS list and the relative values are recalculated amongst the remaining biomarkers on a pro rata basis. That is, if Lactate and Testosterone are the only biomarkers available to be measured, the un-measured biomarkers are removed the IS list and Lactate is assigned an IS of 100*(60/64)=93.75 and Testosterone is reassigned as IS of 100*(4/64)=6.25, ensuring the total weighted scores tally 100.

The IS score serves as a “importance weighting factor” in the calculation of the final Health & Wellness Category Score.

The second aspect to the scoring system involves a Concentration Score (CS) that is defined by the normal range of the biomarker concentration for the subject population (human, canine, bovine, etc.) and then mapped to a range of 0 to 100. CS values closer to 100 represent biomarker concentrations that are within the normal or optimal ranges for that biofluid within the subject population. A CS score further from 100 represents less than optimal concentration of the biofluid. Typically, the optimal CS score is in the middle of the biomarker concentration range with lower scores assigned to both very low or very high biomarker concentrations as these typically indicate poorer health with respect to a biomarker, although this may not always be true. The CS scores can be assigned manually or through extrapolation or fitting according to a number of methods, such as linear regressions, exponential, polynomial, cubic splines, a form of artificial intelligence (AI) assignment or some other method or combination of methods or algorithms. The CS score may or may not be normalized.

The CS score serves as the primary indicator of health for that biomarker.

It is important to note however, that the individual CS assignments may fluctuate due to both subjective and factual input. For example, it may be found that the range of blood serum Lactate is broader or more narrow range or that “normal” may be broader than 0.5-2.3 mM. Further, the assigner may have a bias as it relates to overall range or the definition of a healthy biomarker concentration.

These ranges change by gender and by age. They also vary by weight. Using historical score data and this psychological information, weighting can be calculated using a personalized artificial intelligence algorithm to define personalized weightings.

The final aspect of the scoring system is to combine the IS (Importance Score) and the CS (Concentration Score).

With both the IS and CS scores calculated, they are multiplied given that the CS is the primary health indictor and the IS, in the form of a percentage, is a weighted numeric as it relates to all other underlying biomarkers that serve as indicators of that category's general Health & Wellness.

By way of example, in calculating and Energy Score using only Lactate, Glucose and Testosterone:

The weighted IS scores for each may be Lactate (60), Glucose (30) and Testosterone (10), ensuring that the total IS score of all relevant biomarkers being measured add up to 100.

If the concentration range in human blood serum for Lactate is defined as 0-50 mM, and the range for normal, healthy humans is between 0.5-2.3 mM, a blood serum Lactate concentration of 1.5 mM might receive a CS of 95.

If the concentration range in human blood serum for Glucose is defined as between 0-1,000 mg/dl, and the range for normal, healthy humans is between 70-99 mg/dl, a blood serum Glucose concentration of 130 mg/dl might a CS of 60.

If the concentration range in human blood serum for Testosterone is defined as 0-2,000 ng/dl, and the range for normal, healthy male is between 300-1,000 ng/dl, a blood serum Testosterone concentration of 600 might receive a CS of 80.

Energy Example IS and CS Scores

| Biomarker | IS | CS | Weighted Score |
|---|---|---|---|
| Lactate | 60% | 95 | 57 |
| Glucose | 30% | 60 | 18 |
| Testosterone | 10% | 80 | 8 |
| Totals | 100% | | 83 |

The Energy score for this example is an 83. Typically, the scoring system will assign a higher value (closer to 100) the generally healthier the subject is.

Again, it is important to note that the various ranges that are considered healthy vary from population to population, by gender, age, weight, general fitness levels and can change for an individual over time given their relatively changing fitness levels.

All actual biomarker concentrations, health category scores, and possible actionable insights are transmitted/communicated between the measuring device and mobile software applications, for further action to improve the health category score or share with coaches, medical advisors or others.

FIG. 28B illustrates device 100 communicating, e.g., via Bluetooth, test results and scores to a smart phone according to some embodiments of the present disclosure. The device 100 serves as a biomarker measuring device (left) transmitting Health Category Score (Power) to software application on mobile device (right) via Bluetooth.

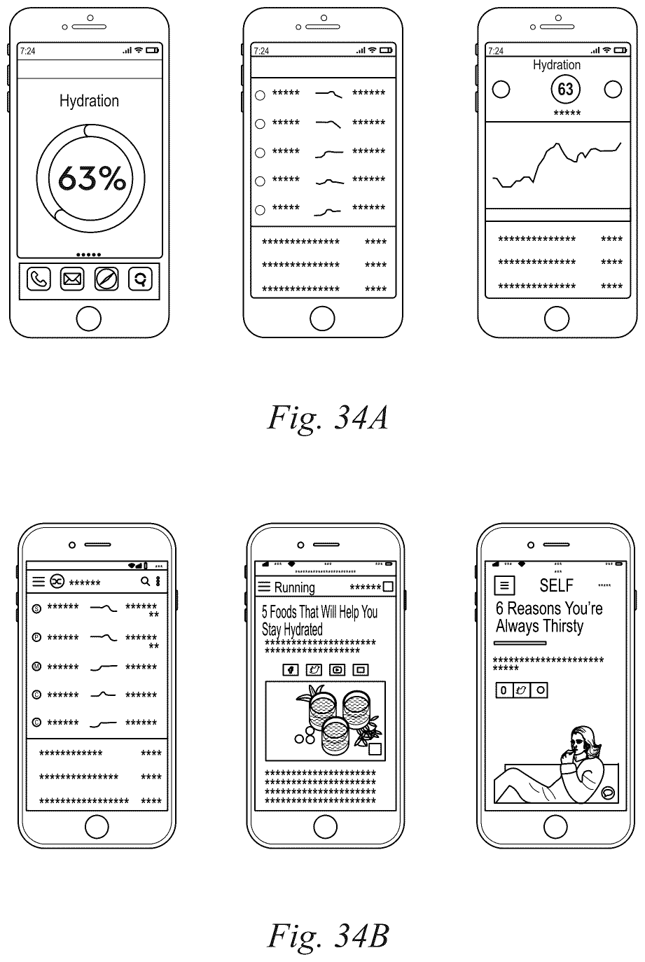

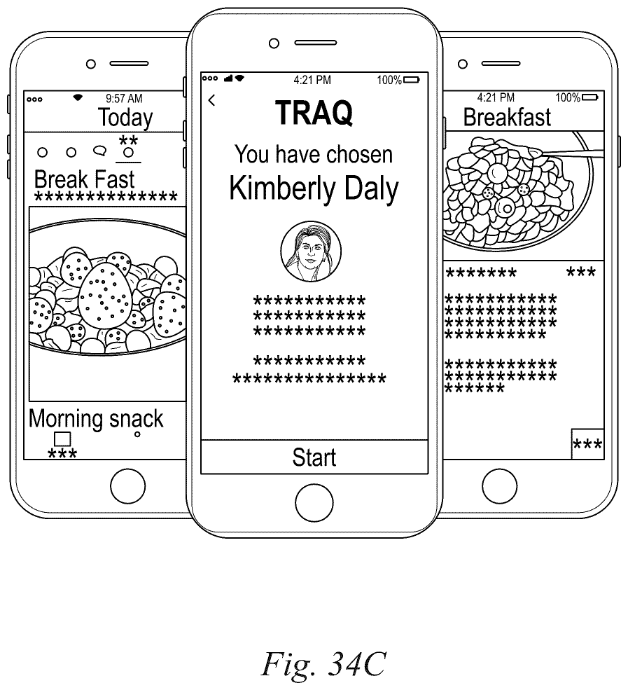

FIG. 34A illustrates some screens on a smartphone showing a health category score for Hydration shown on mobile application (left), along with related underlying biomarker concentrations (middle) and historical scores (right), according to some embodiments of the present disclosure.

FIG. 34B shows a mobile software application displaying underlying biomarker concentration for Hydration heath category (left), and self-directed actionable insights (middle, right) according to some embodiments of the present disclosure.

FIG. 34C shows a mobile software application displaying self-directed actionable insights (left, right) and external link to advisors, coaches, medical professionals or others (center) according to some embodiments of the present disclosure.

According to some embodiments, a saliva sensor or biosensor is provided which may be used with a cartridge 200 and a POCT device 100. The system may be used to test for salivary biomarker(s), as a fitness sensor, health & wellness sensor, as a handheld medical device, and/or for telehealth and/or disease screening.

Biomarkers for different disease states/useful health information are currently obtained using urine or blood samples. Blood sampling is invasive and usually takes sophisticated lab machinery to carry out testing. Consumers cannot easily perform testing using a point of care (POC) device. Such testing is costly, time consuming, and invasive.

Some prior solutions to have included the use of alternative biofluids, such as sweat, tears and saliva. Current salivary testing schema use a mail-in system where the consumer can extract their own saliva and then return the saliva sample to the company for them to run lab diagnostics. These solutions are still time consuming and costly. Despite ease of use, aside from cost and lack of real-time results, as this solution relies on postal delivery and outside lab testing, it also requires knowledge of sophisticated machinery and scientific training, hence no Point-of-Care-Testing (POCT) in real time is available. Lastly, these professionals require safety training and gear given that sample are not self-contained.

According to some embodiments, a point-of-care testing (POCT) device 100 that allows for on-demand, portable, non-invasive (saliva) testing using self-contained, sample collecting cartridges 200 that contain biosensors that enable the measuring a user's fitness, general health & wellness as well as screening for severe maladies is provided. According to some embodiments, results are available in seconds to minutes, and each test costs only a few dollars per test without the need of a medical professional or otherwise.

### 9. Embodiments

Embodiment A1. An electrochemical sensor device for detecting and measuring analyte concentration in saliva, comprising:
a saliva sample collection mechanism comprising a handle and an absorbent swab;
a saliva sample collection reservoir;
a means for computing a general health, fitness or wellness score based partially on analyte concentration;
a means of displaying and communicating analyte concentrations and health, fitness and wellness scores to an external device; and
software that displays same along with self-directed actionable insights and links to external professional or others for additional insight;
wherein the saliva sample collection mechanism and reservoir are disposable after every use.

Embodiment B1. An oral fluid collection device intended for collecting oral fluid comprising:
a handle;
an extendable member;
an absorbent tip; and
a coupling mechanism by which the device can be coupled to a cartridge base.

Embodiment B2. A biofluid collecting device 400 for collecting a biofluid fluid comprising:
a handle 450;
an extendable member 460;
an absorbent tip 480; and
a coupling mechanism 454 by which the device can be coupled to a cartridge base 700.

Embodiment B3. The device of Embodiments B1 or B2 wherein the mechanism is configured to permit the device to be locked to a cartridge base.

Embodiment B4. An biofluid collection device intended for collecting a biofluid comprising:
a handle;
an extendable member; and
an absorbent tip.

Embodiment B5. The biofluid collection device of Embodiment B4 further comprising a coupling mechanism by which the device can be coupled to a cartridge base.

Embodiment B6. The biofluid collection device according to any of Embodiments B2-B4 wherein the biofluid is saliva to be collected by the device from a mouth of a human and wherein the device is configured such that during collection of saliva, the handle remains outside the mouth while the extendable member and the absorbent tip are positioned within the mouth.

Embodiment B7. The fluid collection device of according to any of Embodiments B1-B6 wherein the handle allows for the collection of an oral fluid sample without any oral fluid coming into contact with either the handle itself or a user of the device.

Embodiment B8. The fluid collection device of according to any of Embodiments B1-B7 wherein the extendable member is a telescoping arm.

Embodiment B9. The collection device according to any of Embodiments B1-B8 wherein the extendable member may extend or telescope in such a fashion as to extend the absorbent tip further into an oral cavity for sample collection, and collapse in such a fashion that the device remains more compact when coupled to the cartridge base.

Embodiment B9. The collection device according to any of Embodiments B1-B9 wherein the extendable member has an extended position and a collapsed position and wherein the extendable member extends to its extended position when unrestrained, and collapses to a collapsed position when the absorbent tip and the extendable member are inserted into a cartridge base and the collection device is coupled to the cartridge base.

Embodiment B10. The collecting device according to any of Embodiments B1-B9 wherein the coupling mechanism provides a way by which the extendable member and absorbent tip may be stored in a cartridge base.

Embodiment B11. The collecting device according to any of Embodiments B1-B10 wherein the coupling mechanism allows for the device to be stored in the cartridge base in a compact state, and wherein the coupling mechanism comprises threads to permit the device to be screwed onto a cartridge base or magnets to secure the device to the base magnetically.

Embodiment B12. The collecting device according to any of Embodiments B1-B11 wherein when inserted into a cartridge base, the combination of the device and the base are considered a single cartridge and wherein the device is configured to be seated or locked onto the base in a manner that restricts leakage of any biofluid or oral fluid out of the cartridge.

Embodiment B13. The collection device according to any of Embodiments B1-B12 wherein when the device is inserted into a cartridge base prior to being inserted into an oral cavity, the absorbent tip compresses at the bottom of a chamber in the base in a manner that allows for both closure of the combined device and cartridge base and when the device is removed from the cartridge base, the absorbent tip expands to allow for oral or bio fluid collection.

Embodiment B14. The collection device according to any of Embodiments B1-B13 wherein when the device is inserted into a cartridge base after being inserted into an oral cavity for saliva collection, the absorbent tip compresses at the bottom of a chamber in the cartridge base in a manner that allows for both closure of the combined device and base and the release of collected saliva into a chamber in the base.

Embodiment B15. A method of using the collecting device according to any of Embodiments B1-B14 wherein when the device is inserted into a cartridge base after being inserted into an oral cavity for saliva collection, the absorbent tip is compressed at a bottom of a chamber in the base in a manner that allows for both closure of the combined device and base (cartridge 200) and the release of collected saliva into a chamber of the base.

Embodiment B16. A biofluid collection device intended for collecting a biofluid comprising:
a handle; and
an absorbent tip.

Embodiment B17. The collection device according to any of Embodiments B1-B16 not having any electronics and/or a battery and/or other power source therein.

Embodiment B18. The collection device according to any of Embodiments B1-B16 not having any electronics and/or a battery and/or other power source on a portion thereof configured to be received in a cavity of a body in which a biosample is to be collected.

Embodiment C1. An electrochemically active sensor chip, comprising:
a substrate including an electrically insulative material;
a plurality of electrode wells disposed on the substrate in different locations, wherein each well includes a plurality of electrodes containing a surface including a chemical substance that includes a catalyst or reactant corresponding to an analyte in saliva;
wherein the electrodes are capable of sustaining a redox reaction with the chemical substance and analyte to produce an electrical signal.

Embodiment C2. The sensor chip of Embodiment C1, further comprising:
a spacer layer coupled to the substrate, which allows saliva to pass into the wells; and
a hydrophilic top cover, which directs saliva towards the wells and seals the chip.

Embodiment C3. The sensor chip of Embodiment C1 or C2, wherein two of the electrode wells are used for two-electrode enzymatic analyte analysis.

Embodiment C4. The sensor chip according to any of Embodiments C1-C3, wherein a first well is used for background corrections.

Embodiment C5. The sensor chip according to any of Embodiments C1-C4, wherein a second well is used for electrochemical analysis.

Embodiment C6. The sensor chip according to any of Embodiments C1-05, wherein the wells comprise three electrodes.

Embodiment C7. The sensor chip of Embodiment C6, wherein
a first electrode in each well acts as a sample fill detector;
a second electrode in each well acts as a working electrode; and
a third electrode in each well acts as a reference electrode.

Embodiment C8. The sensor chip according to any of Embodiments C6-C7, wherein the electrodes comprise a conductive material such as carbon ink.

Embodiment C9. The sensor chip of Embodiment C8, wherein the electrodes are configured to be electrically coupled to a reader device via contacts on an edge of the chip.

Embodiment C10. The sensor chip according to any of Embodiments C1-C9, wherein the surfaces of the electrodes in the wells are coated with a polymer film containing an enzyme selective for the specific analyte of interest, a redox mediator, and a surfactant.

Embodiment C11. The sensor chip of Embodiment C10, wherein the enzyme reduces an oxygenated analyte substance in saliva to a second product in a chemical reduction process in which the second product gains electrons; and wherein the enzyme comprises lactate oxidase (LOx), and the analyte comprises lactate.

Embodiment C12. The sensor chip of Embodiment C10, wherein the enzyme comprises glucose oxidase, and the analyte for detection by the device comprises glucose.

Embodiment C13. The sensor chip of Embodiment C10, wherein the enzyme comprises lactose dehydrogenase, and the analyte comprises pyruvate.

Embodiment C14. The sensor chip of Embodiment C10, wherein the enzyme comprises cholesterol oxidase, and the analyte comprises cholesterol.

Embodiment C15. The sensor chip of Embodiment C10, wherein the enzyme comprises ascorbic acid oxidase, and the analyte for detection comprises ascorbic acid.

Embodiment C16. The sensor chip of Embodiment C10, wherein the enzyme comprises alcohol dehydrogenase, and the analyte for detection comprises ethanol.

Embodiment C17. The sensor chip of Embodiment C10, wherein the redox mediator comprises a transition metal complex in which one or more ligands are coordinated to the transition metal; wherein the redox mediator transfers electrons from the enzymatic reduction process to at least one of the electrically conductive electrodes.

Embodiment C18. The sensor chip of Embodiment C17, wherein the transition metal comprises Os, Rh, Ru, Ir, Fe and/or Co.

Embodiment C19. The sensor chip of Embodiment C17, wherein the redox mediator comprises hexaammineruthenium(III) chloride.

Embodiment C20. The sensor chip of Embodiment C17, wherein the redox mediator comprises pentaaminechlororuthenium(III) chloride.

Embodiment C21. The sensor chip of Embodiment C17, wherein the redox mediator comprises ferricyanide chloride.

Embodiment C22. The sensor chip of Embodiment C10, wherein the surfactant facilitates even distribution of film components.

Embodiment C23. The sensor chip of Embodiment C22, wherein the surfactant comprises Triton X-100.

Embodiment C24. The sensor chip of Embodiment C22, wherein the surfactant comprises sodium dodecyl sulfade.

Embodiment C25. The sensor chip of Embodiment C22, wherein the surfactant comprises sodium stearate.

Embodiment C26. The sensor chip of Embodiment C10, wherein the polymer film further comprises a polymer binder.

Embodiment C27. The sensor chip of Embodiment C26, wherein the polymer binder comprises [poly]vinylpyridine (PVP).

Embodiment C28. The sensor chip of Embodiment C26 or C27, wherein the polymer binder comprises trehalose.

Embodiment C29. The sensor chip according to any of Embodiments C26-C29, wherein the polymer binder comprises Methocel.

Embodiment C30. The sensor chip of Embodiment C10, wherein the polymer film is drop cast onto the surface of the electrode wells.

Embodiment C31. The sensor chip of Embodiment C2, wherein the spacer layer comprises an opaque structural layer; wherein the spacer layer further comprises a pressure sensitive adhesive layer on either side.

Embodiment C32. The sensor chip of Embodiment C31, wherein the spacer layer further forms the walls of the electrode wells, and forms space for saliva to flow into the wells.

Embodiment C33. The sensor chip of Embodiment C2, wherein the top cover is coupled to the spacer layer, wherein the top cover comprises a hydrophilic polymer; and the top cover further comprises a pressure sensitive adhesive layer on one side; and the top cover acts as a microfluidic channel moving saliva into the electrode wells through capillary forces.

Embodiment D1. A reader unit comprising:
electrical components for wireless data transmission;
a potentiostat; and
a battery;
wherein the reader unit is configured to collect data, wherein the reader unit is configured to interface with electrodes through contact with a cartridge.

Embodiment D2. A reader unit comprising:
a cartridge receptacle configured to receive therein a cartridge, the cartridge having a biosensor therein, the biosensor configured to generate one or more electrical signals in response to a biofluid contacting the biosensor;
a processor communicatively coupled to the biosensor of a cartridge received in the cartridge receptable and configured to determine concentrations of one or more biomarkers in the biofluid.

Embodiment D3. The reader unit of Embodiment D2 wherein cartridge comprises one or more electrical contacts and the cartridge receptable comprises one or more corresponding electrical contacts configured to contact the electrical contracts of the cartridge when the cartridge is received within the cartridge receptacle thereby permitting the processor to communicate with the biosensor and receive one or more electrical signals from the biosensor.

Embodiment D4. The reader unit of Embodiment D3 wherein the processor determines concentrations of one or more biomarkers in the biofluid using the one or more electrical signals received from the biosensor.

Embodiment D5. The reader unit according to any of Embodiments D2-D4 further comprises a memory communicatively coupled to the processor.

Embodiment D6. The reader unit according to any of Embodiments D1-D5, wherein the unit is a handheld device that performs data analysis and is configured to send data to a phone or display data on an integrated screen.

Embodiment D7. The reader unit according to any of Embodiments D1-D6, wherein the unit comprises a chassis; and the chassis comprises a main body and a release lever.

Embodiment D8. The reader unit of Embodiment D7, wherein the release lever comprises a spring-loaded lever configured to interface with an indentation in a cartridge body of the cartridge; wherein the lever is configured to hold a cartridge within the reader unit and allow for removal of the cartridge from the reader unit.

Embodiment D9. The reader unit of according to any of Embodiments D1-D8, further comprising a printed circuit board (PCB) for performing biosample analysis within the reading unit; and wherein the printed circuit board comprises a processor and a potentiostat; and the potentiostat is configured to perform chronoamperometry or potentiometry on a biofluid sample.

Embodiment D10. The reader unit of Embodiment D9, wherein the processor further comprises logic gated functions to determine when a sample fill is detected in an electrode well of a biosensor.

Embodiment D11. The reader unit of according to any of Embodiments D1-D10, further comprising a wireless antenna for wireless data transfer to a remote device.

Embodiment D12. The reader unit of according to any of Embodiments D1-D11 further comprising a display, wherein the processor is communicatively coupled to the display, wherein the processor can cause data or testing results to be displayed on the display.

Embodiment D13. The reader unit of Embodiment D9, wherein the PCB further comprises a power regulation controller; and the power regulation controller allows for use of the battery; and the battery can be charged through a port in a chassis of the reader.

Embodiment D14. The reader unit according to any of Embodiments D1-D13, wherein a potentiostat is coupled to a biosensor chip by way of internal pins or magnets in a cartridge receptacle of the reader unit; and these pins or magnets are configured to be electrically coupled to contacts on the biosensor chip to complete an electrical circuit used for electrochemical detection.

Embodiment D15. The reader unit according to any of Embodiments D1-D14, wherein the reader comprises a signal conditioning circuit to amplify a detected electrical signal by the biosensor chip.

Embodiment D16. The reader unit according to any of Embodiments D1-D15, further comprising comprises a data processing unit including a processor to process data based on the detected electrical signal and a memory to store or buffer the data.

Embodiment D17. The reader unit according to any of Embodiments D1-D10 wherein the reader unit is a configured to be a handheld unit having a first dimension of less than or equal to about 100 mm and a second, orthogonal dimension of less than or equal to about 50 mm.

Embodiment E1. A cartridge base comprising a biosensor chip according to any of Embodiments C1-C32 configured to interface with a reader unit of according to any of Embodiments D1-D17 and configured to receive saliva a collection device according to any of Embodiments B1-B15 and transfer received saliva to the biosensor chip.

Embodiment E2. A cartridge base comprising a biosensor chip according to any of Embodiments C1-C32 configured to receive saliva a collection device according to any of Embodiments B1-B15 and transfer received saliva to the biosensor chip.

Embodiment E3. The cartridge base of Embodiment E1 or E2 wherein the base is configured to transfer received saliva to the biosensor chip using microfluidic channeling:

Embodiment E4. The cartridge base according to any of Embodiments E1-E3 configured to be inserted into a reader unit, and wherein the reader unit is configured to detect at least one analyte in the received saliva.

Embodiment E5. The cartridge base according to any of Embodiments E1-E4, comprising a cartridge body made of a rigid material; and
wherein the cartridge body has a cylindrical shape having a cavity therein, wherein a first side of the cartridge body has an opening to the cavity; and
wherein the cartridge body comprises a plurality of protrusions located within the cavity,
wherein the protrusions are configured to interface with the saliva collection device;
wherein a portion of the collection device is configured to be inserted in the cavity of the cartridge body,
wherein the protrusions are configured to interface with the collection device in a manner that assists transferring saliva initially on the collection device into the cavity of the cartridge base;
wherein the cartridge base comprises an O-ring within the cavity of the cartridge housing; and
wherein a channel is located within the cavity of the cartridge housing, the channel having an aperture therein permitting saliva to flow to outside of the cavity.

Embodiment E6. The cartridge base of according to any of Embodiments E1-E5, further comprising a sensor cavity configured to house the biosensor chip; wherein the sensor cavity has an indentation which corresponds to the shape of the biosensor chip; and wherein the biosensor chip is secured within the sensor cavity using a latch.

Embodiment E7. The cartridge base according to any of Embodiments E1-E6, further comprising one or more outer indentations that interface with one or more opposite protrusions in a reader unit; and the indentations allow for the cartridge base to be secured within the reader unit during sample analysis and to be released after use by a release latch mechanism on the reader unit.

Embodiment E8. A cartridge base comprising:
a cartridge body defining a collection device cavity therein, wherein a first side of the cartridge body has an opening to the cavity, wherein a portion of a biofluid collection device is configured to be inserted in the collection device cavity of the cartridge body; and
the cartridge body defining a sensor cavity;
a biosensor chip in the sensor cavity.

Embodiment E9. The cartridge base of Embodiment E8, further comprising an aperture extending from a wall of the collection device cavity to a wall of the sensor cavity permitting a biofluid in the collection device cavity to flow onto the biosensor chip in the sensor cavity.

Embodiment E10. The cartridge base according to any of Embodiments E8-E9 wherein the collection device cavity is configured to collect a biofluid along a wall of the collection device cavity transferred from the biofluid collection device when the biofluid collection device is inserted in the collection device cavity.

Embodiment E11. The cartridge base of Embodiment E10, wherein the biofluid collection device comprises an absorbent tip having a biofluid therein and wherein when the absorbent tip is compressed against the wall of the collection device cavity when the biofluid collection device is inserted in the collection device cavity, the biofluid is transferred from the collection device to the wall of the collection device cavity.

Embodiment E12. The cartridge base of Embodiment E11, wherein the cartridge body and the collection device comprise means for securing collection device to the cartridge base within the absorbent tip of the collection device within the collection device cavity.

Embodiment E13. The cartridge base of Embodiment E12, wherein the means for securing the absorbent tip of the collection device within the collection device cavity causes the absorbent tip to be compressed against the wall of the collection device cavity when the collection device is secured to the cartridge base to assist in the transfer of the biofluid from the collection device to the wall of the collection device cavity.

Embodiment E14. The cartridge base according to any of Embodiments E8-E13 not having a battery therein.

Embodiment F1. A method to detect an analyte in saliva comprising:
reducing a chemical substrate in the saliva to a second product in a chemical reduction process in which the second product gains electrons;
supplying the extracted electrical energy to electrodes of an electrochemical sensor; and
detecting, from the electrodes of the electrochemical sensor in contact with the saliva, an electrical signal produced as a result of a redox reaction involving an analyte in the saliva and a chemical agent coupled to at least one of the electrodes of the electrochemical sensor.

Embodiment F2. A method to detect an analyte in saliva comprising:
receiving saliva on electrodes of an electrochemical sensor;
reducing a chemical substrate in the saliva to a second product in a chemical reduction process in which the second product gains electrons;
supplying the extracted electrical energy to the electrodes of the electrochemical sensor; and
detecting, from the electrodes of the activated electrochemical sensor in contact with the saliva, an electrical signal produced as a result of a redox reaction involving an analyte in the saliva and a chemical agent coupled to at least one of the electrodes of the electrochemical sensor.

Embodiment F3. The method of Embodiment F1 or F2 wherein the saliva is transferred to the electrodes via a saliva collection device.

Embodiment F4. The method according to any of Embodiments F1-F3 further comprising collecting the saliva from the mouth of a human using a saliva collection device.

Embodiment F5. The method according to any of Embodiments F1-F4, wherein the electrical signal is detected using amperometry, voltammetry, or potentiometry.

Embodiment F6. The method according to any of Embodiments F1-F5, further comprising processing the electrical signal to determine a parameter of the analyte.

Embodiment F7. The method of Embodiment F6, wherein the parameter includes a concentration level of the analyte.

Embodiment F8. The method according to any of Embodiments F1-F7, further comprising using the electrical signal to quantitatively define health, fitness and diagnostic health measures using various parameters.

Embodiment F9. The method of Embodiment F8, wherein the parameters comprise a detected concentration of one or more biomarkers related to a qualitative health category generally associated with the underlying biofluid.

Embodiment F10. The method of Embodiment F9, wherein the parameters further comprise a weight assigned to each biomarker as it relates to the overall importance to the health category.

Embodiment F11. The method of Embodiment F10, comprising changing the weight assigned to each biomarker based on prior measurements.

Embodiment F12. The method of Embodiment F9, further comprising combining the detected concentration and weights to compute a final health category score.

Embodiment F13. The method of Embodiment F12, wherein the health score can comprise a scaled numeric, binary or other scale to denote a quantitative difference between poor and excellent health.

Embodiment F14. The method of Embodiment F12, further comprising providing actionable insights based on the health score.

Embodiment F15. The method of Embodiment F14, wherein the actionable insights comprise suggestions for dietary or fitness changes to help improve the health score.

Embodiment F16. The method according to any of Embodiments F1-F15, which further comprising using a mobile app running on a remote device.

Embodiment F17. The method of Embodiment F16, further comprising sending, using Bluetooth, data from a reader device receiving the electrical signal to the remote device, and

Embodiment F18. The method of Embodiment F17, wherein the remote device uses the data to perform an analysis.

Embodiment F19. The method of Embodiment F16, wherein the mobile app further determines a health score, provides actionable insights, and/or displays concentrations of measured analytes.

Embodiment G1. A biofluid-based point-of-care testing (POCT) system that provides rapid, real-time results for measuring analytes and biomarkers without requiring expensive blood tests or medical training, for a fraction of the cost of current tests in a handheld device.

Embodiment G2. The system of Embodiment G1 wherein the system allows for the measurement of biomarker or analyte concentration in several biofluids: blood, saliva, tears, sweat or urine. Some embodiments include the use of saliva as the main biofluid, and some or all of the chemistry and techniques described herein. The only differences with other biofluids are in the collection protocol, possible separation techniques and concentration of biomarkers. For blood, the collection protocol may include a pinprick on the finger to draw blood, and this would be deposited onto the cotton collection mechanism and then dispensed into the electrode as usual. The concentration of lactate/other biomarkers in blood is higher than in saliva, so the calibration curves would be adjusted to reflect that. Blood also contains coagulants and other materials that may disturb the analysis, so a filter for bigger particles/blood cells in the test cartridge 200 before the electrode sensor 1300 would allow for accurate measurement. Lesser care would need to be taken for tears, urine and sweat, which share characteristics with saliva. The most important change would be to calibration curves to reflect the concentrations of biomarkers in those fluids.

Embodiment G3. The system of Embodiment G1 wherein the system senses the concentration of biofluid biomarkers with accuracy greater than 90%. According to some embodiments, this is achieved due to the chemistry wherein the redox mediator allows for more efficient transfer of electrons to the working electrode for measurement, and the polymer backbone makeup of a sensing film allows for much higher stability across the usable temperature range. The inclusion of a background correction well 1332 within the sensing array removes any off-target sensitivity.

Embodiment G4. The system of Embodiment G1 wherein the system is configured to aggregate biomarker concentration scores resulting in an average biomarker concentration.

Embodiment G5. The system of Embodiment G1 wherein the system is configured to interface with software applications and databases for display, and/or with storage, analysis and communications means.

Embodiment G6. The system of Embodiment G1 wherein the system is configured to perform artificial intelligence calculations based upon a user's history and current physiological state, and provide recommendations or self-directed, actionable insights focused on improved general or specific health.

Embodiment G7. The system of Embodiment G1 wherein the system is configured to share results and/or history with other devices such as devices (e.g., smartphones or computers) associated with an individual or individuals other than the user. According to some embodiments, such sharing may be done with the intent of seeking input for a user to improve their health or initiating more comprehensive healthcare consultation.

Embodiment H1. A saliva-based point-of-care testing (POCT) system that provides rapid, real-time results for measuring analytes and biomarkers without the use of medical professionals, for a fraction of the cost, in a broad array of demanding physical environments without the use of batteries or power in an in vivo setting.

Embodiment H2. The system of Embodiment H1 wherein the system employs the use of an inexpensive, disposable biofluid or saliva test cartridge 200, and wherein a modular electrode well design of such sensors 1300 allows for cheaply automated drop-casting of the sensor polymer film.

Embodiment H3. The system of Embodiment H1 wherein the system has embedded firmware capable of performing tasks and calculations independently.

Embodiment H4. The system of Embodiment H1 wherein the system allows usage by untrained or trained personnel.

Embodiment H5. The system of Embodiment H1 wherein the system employs the chemical compounds, requiring no refrigeration or special storage mechanisms, to render the device operable.

Embodiment H6. The system of Embodiment H1 wherein the system is capable of delivering robust and stable results over 12 months without special storage or refrigeration.

Embodiment H7. The system of Embodiment H1 wherein the system employs the use disposable and recyclable or upcyclable testing cartridges that allow for a biofluid sampled to be collected without the intervention of medical professionals or scientists utilizing safety equipment or protocols.

Embodiment H8. The system of Embodiment H1 wherein the system employs testing cartridges configured to measure multiple biomarkers or analytes simultaneously.

Embodiment H9. The system of Embodiment H1 wherein the system is configured to aggregate multiple biomarker concentrations and calculate an overall, quantified health, wellness or fitness score.

Embodiment H10. The system of Embodiment H1 wherein the system is configured to scan analytes and biomarkers that serve as indicators of specific temporary or chronic maladies.

Embodiment I1. A saliva-based point-of-care testing (POCT) system 10 that provides rapid, real-time results for measuring analytes and biomarkers related to the scanning of saliva for elevated and sustained levels of lactic acid as it serves as an indicator of serious temporary and chronic maladies.

Embodiment I2. The system of Embodiment I1 wherein the system senses the concentration of biofluid biomarkers with accuracy greater than 10%.

Embodiment 13. The system of Embodiment I1 wherein the system is capable of interfacing with software applications and databases for display, storage and communications means.

Embodiment 14. The system of Embodiment I1 wherein the system is capable of comparing previous scores in improving sensitivity and specificity for lactic acidosis testing.

Embodiment 15. The system of Embodiment I1 wherein the system is capable performing artificial intelligence calculations based upon the user's history and current physiological state, with resulting recommendations or self-directed, actionable insights focused on improved accuracy of acidosis scoring.

Embodiment 16. The system of Embodiment I1 configured to enable results and history to be shared with other individuals with the intent or improving health or initiating more comprehensive healthcare.

Embodiment 17. The system of Embodiment I1 wherein the system operates without the use of medical professionals, for a fraction of the cost, in a broad array of demanding physical environments and without the use of batteries or power in an in vivo setting.

Embodiment 18. The system of Embodiment I1 wherein the system employs the use of inexpensive, manufacturable to scale, and stable electrochemical sensors.

Embodiment 19. The system of Embodiment I1 wherein the system has embedded firmware capable of performing tasks and calculations independently.

Embodiment I10. The system of Embodiment I1 wherein the system allows usage by untrained personnel.

Embodiment I11. The system of Embodiment I1 wherein the system employs the chemical compounds, requiring no refrigeration or special storage mechanisms.

Embodiment I12. The system of Embodiment I1 wherein the system is capable of delivering robust and stable results over 12 months without special storage or refrigeration.

Embodiment I13. The system of Embodiment I1 wherein the system employs a disposable biofluid or saliva test cartridge 200 that are allow for a biofluid sampled to be collected without the intervention of medical professionals or scientists utilizing safety equipment or protocols.

Embodiment I14. The system of Embodiment I1 wherein the system employs a recyclable and/or upcyclable biofluid or saliva test cartridge 200 that are allow for biofluid sampled to be collected without the intervention of medical professionals or scientists utilizing safety equipment or protocols.

Embodiment J1. A system that quantitatively defines typically non-quantified health, fitness and diagnostic health measures using the concentration of related underlying biomarkers within saliva and/or other biofluids (blood, urine, tears, sweat), and a user's general physical parameters, comprising;
a. measuring concentration(s) of various biomarkers related to a qualitative health category generally associated a sample of biofluid;
b. weighting factors assigned to each biomarker as it relates to the overall importance to the qualitative assessment within a health category;
c. an algorithm that combines the concentrations and weighting factors in one of several fashions to compute a final health category score;
d. calculating a final health score that comprises a scaled numeric (e.g. 1-10, or 1-100), binary (e.g. high-low), or any other stratified scale that denotes a quantitative difference between poor health and excellent health (e.g. high-medium-low, green-yellow-red);

Embodiment J2. The system of Embodiment J1 further comprising: an intelligent algorithm that uses scores along with pertinent user health information, to modify weighting factors for a personalized weighting schedule.

Embodiment J3. The system of Embodiment J1, wherein the system is further configured to provide actionable insights related to the health category score that provide the user recommendations on how to improve the overall health category score.

Embodiment J4. The system of Embodiment J1, further comprising an on-device display of underlying biomarker concentrations, health category score and self-directed or external actionable insights that relate to health category score improvement.

Embodiment J5. The system of Embodiment J1, further comprising a mobile software application that displays underlying biomarker concentrations, health category score and actionable insight.

Embodiment J6. The system of Embodiment J1, further comprising a device with a microprocessor and sensing elements capable of measuring biomarker concentrations, storing and altering weighted factors, manually or based on an artificial intelligence algorithm, and calculating final health category score.

Embodiment J7. The system of Embodiment J1, further comprising means of communicating with and transmitting the biomarker concentrations, weight factors and overall health category scores back and forth between a measuring device and a remote device running software application(s) and/or a remote database(s).

Embodiment J8. The system of Embodiment J1, further comprising means of sharing quantitative health category scores with coaches, medical advisors or others, for further actionable insights or counsel.

Embodiment K1. A sample analysis reader configured to be electrically coupled to a sample analysis cartridge, the sample analysis reader comprising:
a processor; and
a non-transitory computer readable medium having instructions stored thereon, wherein the instructions, when executed by the processor, cause the processor to:
   detect a presence of the sample analysis cartridge electrically coupled to the sample analysis reader;
   detect identification information associated with the sample analysis cartridge;
   identify a proper test protocol for the sample analysis cartridge based at least in part on the identification information;
   detect a sample collection device inserted into the sample analysis cartridge; and
   initiate a mixing protocol to mix a sample received form the sample collection device with reagents in fluid in a reservoir in the sample analysis cartridge.

Embodiment K2. The sample analysis reader of Embodiment K1, wherein the processor is configured to initiate the mixing protocol by pumping reagent and sample from respective reservoirs into a mixing reservoir.

Embodiment K3. The sample analysis reader of Embodiment K1, wherein the processor is configured to initiate the mixing protocol to facilitate formation of a plurality of competitive complexes between the analyte present in the saliva and the competitive agent form with a streptavidin-tagged magnetic particle.

Embodiment K4. The sample analysis reader of Embodiment K1, wherein the processor is configured to process one or more signals from one or more electrodes exposed to a bio-sample, the one or more signals being indicative of the quantity of one or more target analytes from the sample.

Embodiment K5. The sample analysis reader of embodiment K1 wherein the sample analysis reader is configured to be electrically coupled to a plurality of sample analysis cartridges that are each disposable.

Embodiment L1. A saliva-based point-of-care testing device (POCT) that provides rapid, real-time results for measuring analytes and biomarkers related to the scanning of saliva for elevated and sustained levels of lactic acid as it serves as an indicator of serious temporary and chronic maladies.

Embodiment L2. The device of Embodiment L1 wherein the device senses the concentration of biofluid biomarkers with an accuracy greater than 90%.

Embodiment L3. The device of Embodiment L1 wherein the device is capable of interfacing with software applications and databases for display, storage and communications means.

Embodiment L4. The device of Embodiment L1 wherein the device is capable of comparing previous scores in improving sensitivity and specificity for lactic acidosis testing.

Embodiment L5. The device of Embodiment L1 wherein the device is capable performing artificial intelligence calculations based upon the user's history and current physiological state, with resulting recommendations or self-directed, actionable insights focused on improved accuracy of acidosis scoring.

Embodiment L6. The device of Embodiment L1 wherein the device is configure to share results and history with other individuals with the intent or improving health or initiating more comprehensive healthcare.

Embodiment L7. The device of Embodiment L1 wherein the device is operated without the use of medical professionals, for a fraction of the cost, in a broad array of demanding physical environments and without the use of batteries or power in an in vivo setting.

Embodiment L8. The device of Embodiment L1 wherein the device employs the use of inexpensive, manufacturable to scale, and stable electrochemical sensors.

Embodiment L9. The device of Embodiment L1 wherein the device has embedded firmware capable of performing tasks and calculations independently.

Embodiment L10. The device of Embodiment L1 wherein the device allows usage by untrained personnel.

Embodiment L11. The device of Embodiment L1 wherein the device employs chemical compounds, requiring no refrigeration or special storage mechanisms.

Embodiment L12. The device of Embodiment L1 capable of delivering robust and stable results over 12 months without special storage or refrigeration.

Embodiment L13. The device of Embodiment L1 wherein the device employs the use disposable and recyclable, upcyclable testing cartridges that are allow for biofluid sampled to be collected without the intervention of medical professionals or scientists utilizing safety equipment or protocols.

Embodiment M1. A point of care salivary testing system comprising:
a biofluid collection device configured to collect a biofluid;
a cartridge base configured to receive the collection device, the cartridge base having a biosensor therein, wherein the cartridge base is configured to transfer collected biofluid from the collection device to the biosensor after the collection device having a collected biofluid thereon is received within the cartridge base, the biosensor having a plurality of electrical pads thereon; and
a reader device configured to receive the cartridge base therein, wherein the reader device has a plurality of electrical contacts configured to become electrically coupled to the electrical pads of the biosensor when the cartridge base is received in the reader device, wherein the system is configured to detect the presence and/or quantity of one or more target analytes in the collected biofluid that has been transfer to the biosensor that is electrically coupled to the electrical contacts of the reader.

Embodiment N1. An electrochemically active sensor chip, comprising:
a substrate including an electrically insulative material; and
a plurality of electrode wells disposed on the substrate in different locations, wherein each well includes a plurality of electrodes containing a surface including a chemical substance that includes a catalyst or reactant corresponding to an analyte in saliva;
wherein the electrodes are capable of sustaining a redox reaction with the chemical substance and analyte to produce an electrical signal.

Embodiment N2. The sensor chip of Embodiment N1 further comprising:
a spacer piece coupled to the substrate, which allows saliva to pass into the wells; and
a hydrophilic top cover, which directs saliva towards the wells and seals the chip.

Embodiment O1. A method of manufacturing a sensing film for a biosensor chip configured to detect the quantity of lactate in a sample comprising:
combining HEPES and BTP in aqueous solvent to make a buffer solution;
dissolving NaCl and TritonX in aqueous solution to make respective stock surfactant solutions;
centrifuging and vortexing PVP and hydroxyethyl cellulose in an aqueous solution to form a polymer solution;
adding Trehalose and sodium succinate to the polymer solution and stirring combination of the Trehalose, sodium succinate, and the polymer solution;
adding the surfactant (TritonX, NaCl) solutions and the polymer solution and centrifuging and vortexing the combined solutions;
adding Methocel to the buffer solution and stirring the combined Methocel/buffer solution for about 1 hour to disperse in solution;
adding Ruthenium hexamine to an aliquot of the polymer solution and stirring the combination for about an hour and then vortexing and centrifuging the combination to generate an intermediate mixture; and
adding LOx to the intermediate mixture and stirring the combination thereof for about 20 minutes to generate a viscous solution.

Embodiment O2. The method of manufacturing a sensing film for a biosensor chip of Embodiment O1 further comprising:

drop casting the viscous solution at about 2 uL droplets into one or more well of the biosensor chip and curing the viscous solution in the one or more wells at about 55 C for about 10 minutes.

Embodiment O3. The method of manufacturing a sensing film for a biosensor chip of Embodiment O2 wherein the sensing film has a self-life of more than 6 months.

Embodiment O4. The method of manufacturing a sensing film for a biosensor chip according to any of Embodiment O2-03 wherein the sensing film yields sensors having reproducibility from sensor to sensor of <5% Lot to Lot: <5%.

Embodiment O5. The method of manufacturing a sensing film for a biosensor chip according to any of Embodiment O2-04 wherein the sensing film yields sensors which may utilize a sample volume of <=about 5 uL.

Embodiment O6. The method of manufacturing a sensing film for a biosensor chip according to any of Embodiment O2-05 wherein the sensing film yields sensors which have a detection time of less than or equal to about 30 seconds.

Embodiment O7. The method of manufacturing a sensing film for a biosensor chip according to any of Embodiment O2-06 wherein the sensing film yields sensors which have a sensitivity of at least about 0.1 mM.

Embodiment O8. The method of manufacturing a sensing film for a biosensor chip according to any of Embodiment O2-07 wherein the sensing film yields sensors which have LOD of at least about 0.2 mM.

Embodiment O9. The method of manufacturing a sensing film for a biosensor chip according to any of Embodiment O2-04 wherein the sensing film yields sensors which have a linearity of at least 3.5 mM.

Embodiment P1. An ion selective hydration sensing film for a sensor, wherein the film may be drop-cast in layers into one or more wells of the sensor, wherein the film coats carbon pads in the one or more wells, the film comprising:
a first layer comprising a hydrogel layer which aids in adhering the sensing film to the carbon pads;
a sensing layer comprising an ionophore and complimentary salt for an ion of interest, which helps generate a membrane potential used for analyzing ion concentration, the sensing layer being applied to the first layer; and
a top layer applied to the sensing layer, the top layer comprising a polymer, which protects the sensing layer from outside interference and damage and adds to overall stability while allowing for diffusion of ions from solution into the sensing layer for use during analysis.

Embodiment P2. The sensing film of Embodiment P1 wherein the top layer comprises PDMS.

Embodiment P3. The sensing film according to any of Embodiments P1-P2 wherein the top layer only contains a polyvinyl alcohol (PVA) film.

Embodiment P4. The sensing film according to any of Embodiments P1-P3 wherein the first layer comprises a polymer film.

Embodiment P5. The sensing film according to any of Embodiments P1-P4 wherein the top layer comprises Dowsil 3140, a form of PDMS.

Embodiment P6. The sensing film according to any of Embodiments P1-P5 wherein the polymer of the top layer is dissolved in a solvent.

Embodiment P7. The sensing film of Embodiment P6 wherein the solvent is selected from the group of THF, 2-Methyltetrahydrofuran, and Cyclopentyl methyl ether.

Embodiment P8. The sensing film according to any of Embodiments P6-P7 wherein a plasticizer is also used in the solvent to drop-cast the polymer of the top layer.

Embodiment P9. The sensing film of Embodiment P8 wherein the plasticizer is selected from the group of DOS, DEHP, adipates, citrates, and phthalates.

Embodiment P10. The sensing film according to any of Embodiments P1-P9 wherein the sensing layer comprises a polymer, ionophore, lipophilic salt and a plasticizer.

Embodiment P11. The sensing film according to any of Embodiments P1-P10 manufactured in the following manner: 2.5% PVA in DI water is prepared, and methanol is added to form a first solution; the first solution is drop-cast into a well of the sensor, and then baked at about 100 C for about 10 minutes to form the first layer;
a second solution of sodium ionophore X, Na-TFPB and THF is stirred for about 30 minutes;
then PVC is added to the second solution and stirred for about 1 hour;
then DOS is added to the second solution and stirred for about 2 hours;
then the second solution is drop-cast into the well of the sensor on top of the first layer and baked at about 40 C for at least 10 hours to form the sensing layer;
a third solution is made up of Dowsil 3140 in THF and DOS, which is stirred for 2 hours;
the third solution is drop-cast on to the sensing layer and left for about an hour at room temperature.

Embodiment Q1. A cartridge comprising:
a cartridge base according to any of Embodiments E1-E14; and
a collection device according to any of Embodiments B1-B18.

Embodiment R1. A system comprising:
a cartridge according to Embodiment Q1; and
a reading unit according to any of Embodiments D1-D17 or K1-K5.

Embodiment R2. The system of Embodiment R1 comprising a sensor chip according to any of Embodiments C1-C33 or N1-N2.

### 10. Conclusion

While the concepts disclosed herein are susceptible to various modifications and alternative forms, specific embodiments thereof have been shown by way of example in the drawings and herein described in detail. It should be understood, however, that it is not intended to limit the inventions to the particular forms disclosed, but on the contrary, the intention is to cover all modifications, equivalents, and alternatives falling within the spirit and scope of the inventions as defined by the appended claims.

What is claimed is:

1. A point of care salivary testing system comprising:
a biofluid collection device configured to collect a biofluid;
a cartridge base configured to receive the collection device, the cartridge base having a biosensor therein, wherein the cartridge base is configured to transfer collected biofluid from the collection device to the biosensor after the collection device having a collected biofluid thereon is received within the cartridge base, the biosensor having a plurality of electrical pads thereon; and
a reader device configured to receive the cartridge base therein, wherein the reader device has a plurality of electrical contacts configured to become electrically coupled to the electrical pads of the biosensor when the cartridge base is received in the reader device, wherein the system is configured to detect the presence and/or quantity of one or more target analytes in the collected biofluid that has been transfer to the biosensor that is electrically coupled to the electrical contacts of the reader.

2. An biofluid collection device intended for collecting a bio fluid comprising:
a handle;
an extendable member; and
an absorbent tip.

3. The biofluid collection device of claim 2 further comprising a mechanism by which the device can be coupled to a cartridge base.

4. The biofluid collection device of claim 2 wherein the biofluid is saliva to be collected by the device from a mouth of a human wherein the device is configured such that during collection of saliva, the handle remains outside the mouth while the extendable member and the absorbent tip are positioned within the mouth.

5. An electrochemically active sensor chip, comprising:
a substrate including an electrically insulative material; and
a plurality of electrode wells disposed on the substrate in different locations, wherein each well includes a plurality of electrodes containing a surface including a chemical substance that includes a catalyst or reactant corresponding to an analyte in saliva;
wherein the electrodes are capable of sustaining a redox reaction with the chemical substance and analyte to produce an electrical signal.

6. The sensor chip of claim 5 further comprising:
a spacer piece coupled to the substrate, which allows saliva to pass into the wells; and
a hydrophilic top cover, which directs saliva towards the wells and seals the chip.

7. An electrochemically active sensor chip, comprising:
a plurality of electrode wells, wherein each well includes a plurality of electrodes containing a surface including a chemical substance that includes a catalyst or reactant corresponding to an analyte in a biosample;
wherein the electrodes are capable of sustaining a redox reaction with the chemical substance and analyte to produce an electrical signal in response to the biosample contacting the electrodes.

8. An ion selective hydration sensing film for a sensor, wherein the film may be drop-cast in layers into one or more wells of the sensor, wherein the film coats carbon pads in the one or more wells, the film comprising:
a first layer comprising a hydrogel layer which aids in adhering the sensing film to the carbon pads;
a sensing layer comprising an ionophore and complimentary salt for an ion of interest, which helps generate a membrane potential used for analyzing ion concentration, the sensing layer being applied to the first layer; and
a top layer applied to the sensing layer, the top layer comprising a polymer, which protects the sensing layer from outside interference and damage and adds to overall stability while allowing for diffusion of ions from solution into the sensing layer for use during analysis.

9. A method of manufacturing a sensing film for a biosensor chip configured to detect the quantity of lactate in a sample comprising:
combining HEPES and BTP in aqueous solvent to make a buffer solution;
dissolving NaCl and TritonX in aqueous solution to make respective stock surfactant solutions;
centrifuging and vortexing PVP and hydroxyethyl cellulose in an aqueous solution to form a polymer solution;
adding Trehalose and sodium succinate to the polymer solution and stirring combination of the Trehalose, sodium succinate, and the polymer solution;
adding the surfactant (TritonX, NaCl) solutions and the polymer solution and centrifuging and vortexing the combined solutions;
adding Methocel to the buffer solution and stirring the combined Methocel/buffer solution for about 1 hour to disperse in solution;
adding Ruthenium hexamine to an aliquot of the polymer solution and centrifuging and vortexing the combination and stirring the combination for about an hour to generate an intermediate mixture; and
adding LOx to the intermediate mixture and stirring the combination thereof for about 20 minutes to generate a viscous solution.

10. A reader unit comprising:
electrical components for wireless data transmission;
a potentiostat; and
a battery;
wherein the reader unit is configured to collect data,
wherein the reader unit is configured to interface with electrodes through contact with a cartridge.

11. A reader unit comprising:
a cartridge receptacle configured to receive therein a cartridge, the cartridge having a biosensor therein, the biosensor configured to generate one or more electrical signals in response to a biofluid contacting the biosensor;
a processor communicatively coupled to the biosensor of a cartridge received in the cartridge receptable and configured to determine concentrations of one or more biomarkers in the biofluid.

12. A sample analysis reader unit configured to be electrically coupled to a sample analysis cartridge, the sample analysis reader comprising:
a processor; and
a non-transitory computer readable medium having instructions stored thereon, wherein the instructions, when executed by the processor, cause the processor to:
detect a presence of the sample analysis cartridge electrically coupled to the sample analysis reader;
detect identification information associated with the sample analysis cartridge;
identify a proper test protocol for the sample analysis cartridge based at least in part on the identification information;
detect a sample collection device inserted into the sample analysis cartridge; and
initiate a mixing protocol to mix a sample received form the sample collection device with reagents in fluid in a reservoir in the sample analysis cartridge.

13. A saliva-based reading unit for measuring analytes and biomarkers related to the scanning of saliva for elevated and sustained levels of lactic acid as it serves as an indicator of serious temporary and chronic maladies.

14. A point of care salivary testing system comprising:
a biofluid collection device configured to collect a biofluid;
a cartridge base configured to receive the collection device, the cartridge base having a biosensor therein, wherein the cartridge base is configured to transfer collected biofluid from the collection device to the biosensor after the collection device having a collected biofluid thereon is received within the cartridge base, the biosensor having a plurality of electrical pads thereon; and
a reader device configured to receive the cartridge base therein, wherein the reader device has a plurality of electrical contacts configured to become electrically coupled to the electrical pads of the biosensor when the cartridge base is received in the reader device, wherein the system is configured to detect the presence and/or quantity of one or more target analytes in the collected biofluid that has been transfer to the biosensor that is electrically coupled to the electrical contacts of the reader.

15. A method to detect an analyte in saliva comprising:
reducing a chemical substrate in the saliva to a second product in a chemical reduction process in which the second product gains electrons;
supplying the extracted electrical energy to electrodes of an electrochemical sensor; and
detecting, from the electrodes of the electrochemical sensor in contact with the saliva, an electrical signal produced as a result of a redox reaction involving an analyte in the saliva and a chemical agent coupled to at least one of the electrodes of the electrochemical sensor.

16. A method to detect an analyte in saliva comprising:
receiving saliva on electrodes of an electrochemical sensor;
reducing a chemical substrate in the saliva to a second product in a chemical reduction process in which the second product gains electrons;
supplying the extracted electrical energy to the electrodes of the electrochemical sensor; and
detecting, from the electrodes of the activated electrochemical sensor in contact with the saliva, an electrical signal produced as a result of a redox reaction involving an analyte in the saliva and a chemical agent coupled to at least one of the electrodes of the electrochemical sensor.

17. A system that quantitatively defines typically non-quantified health, fitness and diagnostic health measures using the concentration of related underlying biomarkers within saliva and/or other biofluids (blood, urine, tears, sweat), and a user's general physical parameters, comprising;
a. measuring concentration(s) of various biomarkers related to a qualitative health category generally associated a sample of biofluid;
b. weighting factors assigned to each biomarker as it relates to the overall importance to the qualitative assessment within a health category;
c. an algorithm that combines the concentrations and weighting factors in one of several fashions to compute a final health category score;
d. calculating a final health score that comprises a scaled numeric (e.g. 1-10, or 1-100), binary (e.g. high-low), or any other stratified scale that denotes a quantitative difference between poor health and excellent health (e.g. high-medium-low, green-yellow-red).

18. A biofluid testing system comprising:
a biofluid collection device configured to collect a biofluid;
a cartridge base configured to receive the collection device, the cartridge base having a biosensor therein, wherein the cartridge base is configured to transfer collected biofluid from the collection device to the biosensor after the collection device having a collected biofluid thereon is received within the cartridge base, the biosensor having a plurality of electrical pads thereon; and
a reader device configured to receive the cartridge base therein, wherein the reader device has a plurality of electrical contacts configured to become electrically coupled to the electrical pads of the biosensor when the cartridge base is received in the reader device, wherein the system is configured to detect the presence and/or quantity of one or more target analytes in the collected biofluid that has been transfer to the biosensor that is electrically coupled to the electrical contacts of the reader

19. The system of claim 18 wherein the biosensor comprises:
a substrate including an electrically insulative material; and
a plurality of electrode wells disposed on the substrate in different locations, wherein each well includes a plurality of electrodes containing a surface including a chemical substance that includes a catalyst or reactant corresponding to an analyte in saliva;
wherein the electrodes are capable of sustaining a redox reaction with the chemical substance and analyte to produce an electrical signal.

20. The system of claim 20 wherein the biosensor further comprises:
a spacer piece coupled to the substrate, which allows saliva to pass into the wells; and
a hydrophilic top cover, which directs saliva towards the wells and seals the sensor.
